(19) United States
(12) Patent Application Publication
(10) Pub. No.: US 2022/0081721 A1
(43) Pub. Date: Mar. 17, 2022

(54) CHARACTERIZATION OF BONE MARROW USING CELL-FREE MESSENGER-RNA

(71) Applicant: Molecular Stethoscope, Inc., San Diego, CA (US)

(72) Inventors: Michael Nerenberg, Del Mar, CA (US); Arkaitz Ibarra, San Diego, CA (US); Jiali Zhuang, San Diego, CA (US)

(21) Appl. No.: 17/242,137

(22) Filed: Apr. 27, 2021

Related U.S. Application Data
(63) Continuation of application No. PCT/US2019/058380, filed on Oct. 28, 2019.
(60) Provisional application No. 62/752,155, filed on Oct. 29, 2018.
(60) Provisional application No. 62/818,603, filed on Mar. 14, 2019.

(51) Int. Cl. C12Q 1/6886; A61K 45/06; A61K 35/28; C12N 5/077

(52) U.S. Cl. CPC C12Q 1/6886; A61K 45/06; C12N 2501/22; C12N 5/0669; C12N 2501/14; A61K 35/28

(57) ABSTRACT

Described herein are methods and systems for monitoring a disease state of a subject's bone marrow. Further, disclosed herein are methods and systems for monitoring a treatment state of a subject's organ. Moreover, disclosed herein are methods and systems for monitoring a healthy state of a subject's bone marrow and assaying an active agent.

### CROSS-REFERENCE

This application claims the benefit of U.S. Provisional Application No. 62/752,155, filed on Oct. 29, 2018, and U.S. Provisional Application No. 62/818,603, filed on Mar. 14, 2019, each of which is entirely incorporated herein by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Blood is a liquid connective tissue that irrigates all organs, supplying oxygen and nutrients to the cells of the body while collecting their waste, including lipids, proteins, and nucleic acids. These circulating biomolecules contain information linked to specific organ health. While research has focused on circulating proteins and lipids, circulating cell-free DNA (cfDNA) has also emerged as a non-invasive tool for diagnosis and monitoring of health and disease. For example, cfDNA has been utilized for prenatal diagnostics, transplant rejection, and monitoring of cancer. Despite these advances, the value of cfDNA tests is generally restricted to physiologic and disease situations characterized by genetic differences (i.e., pregnancy, transplants, or tumors). For RNA-based non-invasive biomarkers, non-coding RNAs including miRNA and lncRNA have been studied in multiple diseases.

### SUMMARY

In an aspect, presented herein are methods for monitoring a disease state of a subject's bone marrow. The methods comprise obtaining a biological sample from the subject having the disease state; and detecting cell-free mRNA (cf-mRNA) levels of a first plurality of cf-mRNAs derived from a plurality of cells resident or originated from the bone marrow corresponding to a first plurality of genes.

In some embodiments, the biological sample comprises a blood sample. In some embodiments, the blood sample comprises a serum sample, a plasma sample, or a buffy coat sample.

In some embodiments, the disease state comprises multiple myeloma (MM), leukemia, myeloproliferative neoplasms, myelodysplastic syndrome, lymphoma, thrombocythemia, myelofibrosis, polycythemia vera or anemia. In some embodiments, the disease state comprises MM. In some embodiments, when the disease state comprises MM, the first plurality of genes comprises IGHG1, IGHA1, IGKC, IGHV1, IGHV2, IGHV3, IGHV4, IGHV5, IGHV6, IGHV7, IGHV8, IGHV9, IGHV10, IGHV11, IGHV12, IGHV13, IGHV14, IGHV15, IGHV16, IGHV17, IGHV18, IGHV19, IGHV20, IGHV21, IGHV22, IGHV23, IGHV24, IGHV25, IGHV26, IGHV27, IGHV28, IGHV29, IGHV30, IGHV31, IGHV32, IGHV33, IGHV34, IGHV35, IGHV36, IGHV37, IGHV38, IGHV39, IGHV40, IGHV41, IGHV42, IGHV43, IGHV44, IGHV45, IGHV46, IGHV47, IGHV48, IGHV49, IGHV50, IGHV51, IGHV52, IGHV53, IGHV54, IGHV55, IGHV56, IGHV57, IGHV58, IGHV59, IGHV60, IGHV61, IGHV62, IGHV63, IGHV64, IGHV65, IGHV66, IGHV67, IGHV68, IGHV69, IGKV2, IGKV3, IGKV4, IGKV5, IGKV6, IGKV7, IGKV8, IGKV9, IGKV10, IGKV11, IGKV12, IGKV13, IGKV14, IGKV15, IGKV16, IGKV17, IGKV18, IGKV19, IGKV20, IGKV21, IGKV22, IGKV23, IGKV24, IGL1, IGLV 1-40, or a combination thereof. In some embodiments, the disease state comprises acute myeloid leukemia (AML).

In some embodiments, the detecting further comprises converting a cf-mRNA to a cDNA. In some embodiments, the methods further comprise measuring the cDNA by performing one or more of sequencing, array hybridization, or nucleic acid amplification.

In some embodiments, the methods further comprise providing a treatment. In some embodiments, the treatment comprises ionizing irradiation, melphalan-mediated bone marrow ablation, busulfan-mediated bone marrow ablation, treosulfan-mediated ablation, chemotherapy-mediated ablation, allogeneic transplant, autologous transplant, stimulation with growth factors, autologous or heterologous CAR-T cell therapy, or any combination thereof. In some embodiments, the stimulation with growth factors comprises stimulation with erythropoietin (EPO). In some embodiments, the stimulation with growth factors comprises simulation with granulocyte colony stimulating factor (G-CSF).

In another aspect, disclosed herein are methods for monitoring a treatment state of a subject's organ. The methods comprise obtaining a plasma sample from the subject having the treatment state; and detecting cell-free mRNA (cf-mRNA) levels of a second plurality of cf-mRNAs derived from the subject's organ corresponding to a second plurality of genes.

In some embodiments, the organ is bone marrow. In some embodiments, the biological sample comprises a blood sample. In some embodiments, the blood sample comprises a serum, plasma sample or a buffy coat sample.

In some embodiments, the treatment state comprises bone marrow ablation, bone marrow reconstitution, bone marrow transplant, stimulation with growth factors, immunotherapy, immunomodulation, modulation of ubiquitin ligase activities, corticosteroids, radiation therapy, or autologous or heterologous CAR-T cell therapy. In some embodiments, the modulation of the ubiquitin ligase activities comprises administering a ubiquitin ligase inhibitor. In some embodiments, the bone marrow ablation comprises physical ablation, chemical ablation, or a combination thereof. In some embodiments, the physical ablation comprises ionizing irradiation.

In some embodiments, the chemical ablation comprises melphalan-mediated bone marrow ablation, busulfan-mediated bone marrow ablation, treosulfan-mediated ablation, chemotherapy-mediated ablation, or a combination thereof. In some embodiments, the bone marrow transplant comprises allogeneic transplant. In some embodiments, the bone marrow transplant comprises autologous transplant. In some embodiments, the stimulation with growth factors comprises stimulation with erythropoietin (EPO). In some embodiments, the stimulation with growth factors comprises simulation with granulocyte colony stimulating factor (G-CSF).

In some embodiments, when the treatment comprises bone marrow ablation, levels of the second plurality of cf-mRNAs corresponding to the second plurality of genes are decreased, and the second plurality of genes comprises erythrocyte-specific genes.

In some embodiments, when the treatment comprises bone marrow reconstitution, levels of the second plurality of cf-mRNAs corresponding to the second plurality of genes are increased compared to such cf-mRNA levels during bone marrow ablation, and the second plurality of genes comprises erythrocyte-specific genes. In some embodiments, the erythrocyte-specific genes comprises one or more genes from the group consisting of GATA1, SLC4A1, TF, AVP, RUNDC3A, SOX6, TSPO2, HBZ, TMCC2, SELENBP1, ALAS2, EPB42, GYPA, C17orf99, HBA2, RHCE, HBG2, TRIM10, HBA1, HBM, HBG1, UCA1, GYPB, CTD-3154N5.2, and AC104389.1.

In some embodiments, when the treatment comprises bone marrow reconstitution, levels of the second plurality of cf-mRNAs corresponding to the second plurality of genes are increased, and the second plurality of genes comprises megakaryocyte-specific genes. In some embodiments, the megakaryocyte-specific genes comprises one or more genes from the group consisting of ITGA2B, RAB27B, GUCY1B3, GP6, HGD, PF4, CLEC1B, CMTM5, GP9, SELP, DNM3, LY6G6F, LY6G6D, XXbac-BPG3213.19, and RP11-879F14.2.

In some embodiments, when the treatment comprises bone marrow ablation, levels of the second plurality of cf-mRNAs corresponding to the second plurality of genes are decreased, and the second plurality of genes comprises neutrophil-specific genes.

In some embodiments, when the treatment comprises bone marrow transplant, levels of the second plurality of cf-mRNAs corresponding to the second plurality of genes are increased compared to such cf-mRNA levels during bone marrow ablation, and the second plurality of genes comprises neutrophil-specific genes.

In some embodiments, when the treatment comprises bone marrow reconstitution, levels of the second plurality of cf-mRNAs corresponding to the second plurality of genes are increased compared to such cf-mRNA levels during bone marrow reconstitution, and the second plurality of genes comprises neutrophil-specific genes. In some embodiments, the neutrophil-specific genes comprise progenitor-neutrophil-specific genes. In some embodiments, the progenitor-neutrophil-specific genes comprise CTSG, ELANE, AZU1, PRTN3, MMP8, RNASE, PGLYRP1, or a combination thereof. In some embodiments, the detected cf-mRNAs corresponding to progenitor-neutrophil-specific genes appear earlier than a plurality of neutrophil cells in the blood sample.

In some embodiments, when the treatment comprises allogeneic transplant, levels of the second plurality of cf-mRNAs corresponding to the second plurality of genes are detected, and the second plurality of genes comprises progenitor-neutrophil-specific genes from a donor cell.

In some embodiments, when the treatment comprises simulation with G-CSF, levels of the second plurality of cf-mRNAs corresponding to the second plurality of genes are detected, and the second plurality of genes comprises neutrophil-specific genes. In some embodiments, the neutrophil-specific genes comprise one or more genes from the group consisting of PGLYRP1, LTF, ATP2C2, VNN3, CRISP3, CTSG, OLFM4, KRT23, MMP8, ARG1, EPX, PI3, CRISP2, STEAP4, LCN2, PRG3, KCNJ15, ALPL, FCGR38, S100A12, PROK2, CXCR1, CAMP, RNASE3, CEACAM3, AZU1, ABCA13, CXCR2, CTD-3088G3.8, PRTN3, ELAINE, CD177, LINC00671, ORM2, ORM1, HP, and RP11-678G14.4.

In another aspect, disclosed herein are methods for monitoring a healthy state of a subject's bone marrow. The methods comprise obtaining a biological sample from the subject having the healthy state; and detecting cell-free mRNA (cf-mRNA) levels of a third plurality of cf-mRNAs derived from the subject's bone marrow and derived cells thereof corresponding to a third plurality of genes.

In some embodiments, the third plurality of genes comprises about at least 45%, 55%, 65%, or 75% of genes derived from bone marrow and derived cells thereof. In some embodiments, the third plurality of genes comprises one or more genes from Table 7. In some embodiments, the levels of the third plurality cf-mRNA corresponding to progenitor-neutrophil-specific genes are increased compared to cf-mRNA levels corresponding to mature neutrophil-specific genes.

In some embodiments, the biological sample comprises a blood sample. In some embodiments, the blood sample comprises a serum sample, a plasma sample, or a buffy coat sample. In some embodiments, the detecting further comprises converting a cf-mRNA to a cDNA. In some embodiments, the methods further comprise measuring the cDNA by performing one or more of sequencing, array hybridization, or nucleic acid amplification.

In another aspect, disclosed herein are methods for assaying an active agent. The methods comprise assessing a first cell-free expression profile of a subject at a first time point; administering an active agent to the subject; and assessing a second cell-free expression profile of the subject at a second time point.

In some embodiments, either the first or the second cell-free expression profile is bone marrow specific. In some embodiments, the methods further comprise comparing the first cell-free expression profile to the second cell-free expression profile.

In some embodiments, a difference between the first expression profile and the second expression profile indicates an effect of the therapy. In some embodiments, the active agent comprises a pharmaceutical compound to treat a disease.

In some embodiments, the methods further comprise assessing a third cell-free expression profile of the subject at a third time point. In some embodiments, the assessing comprises one or more of sequencing, array hybridization, or nucleic acid amplification. In some embodiments, the methods further comprise assessing additional cell-free expression profiles of the subject at additional time points.

In some embodiments, the second time point is from one to four weeks after the first time point. In some embodiments, the methods further comprise assessing the additional cell-free expression time points over a period of from 12 to 24 months. In some embodiments, the period is about 18 months.

In some embodiments, the methods further comprise tracking and/or detecting one or more cell-free expression profiles to measure one or more targets of interest for therapy and/or drug discovery and/or development. In some embodiments, the methods further comprise measuring pharmacodynamics for a lead optimization and/or a clinical development during therapy and/or drug discovery and development.

In some embodiments, the methods further comprise creating a profile of gene expression to characterize one or more pharmacodynamic effects associated with an engagement of a specific target for therapy and/or drug discovery and/or development. In some embodiments, the methods further comprise detecting changes in pharmacodynamics target engagement for therapy and/or drug discovery and development.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1G show that cf-mRNA transcriptome is enriched in immature hematopoietic transcripts from the bone marrow compared to circulating blood cells; left panels of FIG. 1A show cf-mRNA transcriptome and whole blood transcriptome from healthy subjects was decomposed using non-negative matrix factorization and tissue contribution estimated using public databases. Cf-mRNA was sequenced from 24 normal donors and whole blood RNA-Seq data from 19 healthy individuals was obtained from Whole blood gene expression in adolescent chronic fatigue syndrome: an exploratory cross-sectional study suggesting altered B cell differentiation and survival. J Transl Med. 2017; 15(1):102 (incorporated herein in its entirety). Estimated contribution of the indicated cell types/tissues for each sample is shown. Right panel, average values for each bio fluid (24 cf-mRNA and 19 whole blood samples) are shown. FIG. 1B shows that RNA-seq was performed in 3 paired plasma and whole blood samples from healthy individuals. Levels of indicated cell type-specific transcripts were compared between cf-mRNA and whole blood for all 3 donors. Average fold change (cf-mRNA/whole blood) among the 3 individuals is represented (log scale) (p-value, Wilcoxon test). Dots on the left, neutrophil progenitor transcripts. Dots on the right, mature neutrophil transcripts. Cell type specific genes were identified as explained in examples. See also Table 7. FIG. 1C shows that RNA-seq was performed in 5 paired plasma and buffy coat samples from healthy individuals. Levels of mature and progenitor neutrophil transcripts in plasma and matching buffy coat specimens were compared. Average fold change of these transcripts (plasma/buffy coat) in the five paired samples is shown (log scale). p-value, Wilcoxon test. FIGS. 1D-1E show box-plot comparing the normalized levels (TPM) of the indicated transcripts in paired buffy coat and cf-mRNA samples measured by RNA-Seq (n=5, p-value: Wilcoxon test), showing that cf-mRNA is enriched in immature (PRTN3) hematopoietic transcripts (E) and depleted of mature transcripts (CXCR2, D). Boxes map median, 25th and 75th quintiles, and the whiskers extend to 1.5× interquartile range (IQR). FIG. 1F shows that scatter plot comparing the levels in matching cf-mRNA (Y axis) and whole blood (X axis) of BM-specific genes (in a solid-line circle) and peripheral blood-specific genes (in a dotted line circle), which form two distinct populations (p<0.001), and where bone marrow specific genes are enriched in the cf-mRNA fraction (See also FIGS. 6A-6F). FIG. 1G shows fraction of transcripts listed in FIG. 1A.

FIGS. 2A-2D show cf-mRNA transcriptome captures Ig transcripts derived from the BM of Multiple Myeloma patients. FIG. 2A shows that matching cf-mRNA and buffy coat samples from a Multiple Myeloma patient before BM ablation (day−2) were analyzed by RNA-Seq. Fraction of transcripts from the variable regions of the immunoglobulin heavy and light chains identified in plasma and buffy coat samples are shown (center and right panels). Clonally amplified transcripts are indicated in the patterned portion and dominated the cf-mRNA of the MM Patient. Levels of Ig transcripts in plasma of a healthy individual (left panel) are shown as reference. FIG. 2B shows schematic of the therapeutic treatment performed in MM patients. Melphalan-mediated BM ablation started at day −2, autologous stem cell transplant was performed at day 0. Steroids and G-CSF were then administered as supportive care. Blood was collected every day during the study. FIG. 2C shows bar graphs showing the normalized values (TPM, Y axis) of Ig transcripts detected by RNA-Seq in paired plasma and buffy coat samples throughout the treatment. The repertoire of variable regions of Ig heavy chain and Ig Kappa light chain are shown in a color gradient. Dominant transcripts identified in plasma are indicated. Day of blood collection with respect to transplant is indicated in the X axis. FIG. 2D shows fraction of transcripts from variable Ig regions in cf-mRNA during BM ablation and transplant. Day of blood collection with respect to transplant is indicated in the X axis. Dominant Ig transcripts, shown in solid lines labeled with IGKV2-24 and IGH3-15 respectively, decrease after Melphalan-mediated BM ablation. (See also FIGS. 7A-7C).

FIGS. 3A-3J show cf-mRNA reflects the transcriptional activity of hematopoietic lineages during BM ablation and reconstitution in cancer patients. FIGS. 3A and 3B show heat map of time-varying transcripts identified by cf-mRNA-Seq on multiple myeloma (MM) (A) and acute myeloid leukemia (AML) (B) patients undergoing BM ablation followed by autologous or allogenic stem cell transplant respectively (at day 0). Each column represents a time point with respect to the time of transplant, indicated in the bottom. Each row represents a gene. Enriched gene ontology terms for each cluster of transcripts are indicated (adjusted p value). FIGS. 3C-3H show time course of the levels of erythrocyte (solid-line, C, D), megakaryocyte (solid-line, E, F) and neutrophil (solid-line, G, H) specific transcripts in MM (C, E, G) and AML (D, E, H) patients throughout the study. Transcript identity is provided in Table S3. Corresponding peripheral blood counts are plotted in the secondary axis and represented with a black dotted line (RBC count, millions per mL (C, D), platelet count, thousands per mL (E, F) and neutrophil count, thousands per mL (G, H). Day of blood collection with respect to transplant is indicated in the X axis. FIGS. 3I-3J show relative variation of progenitor neutrophil transcripts in AML patients 1 (I) and 2 (J) throughout the study. Average percent change for these transcripts is represented with a dashed blue line. Dashed black line shows neutrophil counts in blood. In both patients, during BM reconstitution progenitor neutrophil transcripts recovery in plasma precedes neutrophil count.

FIGS. 4A-4E show monitoring of BM allotransplant engraftment in AML patients by genetic differences in cf-mRNA. FIG. 4A shows average frequency of reference allele of the SNPs detected in ELANE, AZU1 and PRTN3 neutrophil progenitor transcripts in cf-mRNA before and after allogeneic HSC transplantation in 3 AML patients, showing implantation of a new genetic profile after transplant. FIGS. 4B and 4C show frequency of reference allele of the SNPs detected in the same transcripts than in (A) for AML Patients 1 and 2. Day of blood collection with respect to the time of transplant is indicated in the X axis. FIGS. 4D and 4E show average reference allele frequency of all SNPs detected in the host cf-mRNA changing from reference homozygous to heterozygous (D) and from alternative homozygous to reference homozygous (E) after transplant. Day of blood collection is indicated in the X axis, transplant occurred at day 0.

FIGS. 5A-5D show cf-mRNA captures the transcriptional activity of hematopoietic lineages upon stimulation. FIG. 5A shows blood was obtained from 9 patients before (day 0) and after (day 3, 4) being treated with a single EPO dose. Gene expression patterns in cf-mRNA were analyzed using RNA-Seq. Day 0 (before EPO treatment) was used as reference for each Patient, and changes in the levels of erythrocyte-specific transcripts after EPO treatment calculated. Average fold change of erythrocyte transcripts in all 9 patients subjected to EPO treatment and 2 untreated controls are shown. Error bars represent standard error (SE). FIG. 5B shows time course analysis of erythrocyte transcripts over a 30-day period in EPO treated patients. Each line represents a patient, and shows average fold change of erythrocyte transcripts over time after a single EPO dosing administered at day 0, which is used as reference. Solid lines around the dashed line labeled mature show fluctuations of the same transcripts in untreated healthy controls. See also FIG. 10. FIG. 5C shows blood was obtained from 3 healthy patients treated with G-CSF (before treatment (day 0), and 1, 4 and 10 days after treatment). Changes in circulating transcriptome were analyzed by RNA-seq in plasma. Relative changes of immature and mature neutrophil specific transcripts throughout the study are shown for a representative patient treated with G-CSF. Dashed line labeled immature and dashed line labeled mature indicate the average for each group of transcripts. Relative changes in neutrophil counts are shown in black. FIG. 5D shows time course of indicated G-CSF responsive genes measured by cf-mRNA-Seq. Plots show fold change over time relative to day 0. Time points are connected by lines, each line represent a patient. See also FIG. 10.

FIGS. 6A-6F show cf-mRNA transcriptome is enriched in bone marrow transcripts compared to circulating cell transcriptome. FIG. 6A is a schematic of whole blood, plasma and buffy coat composition. FIGS. 6B and 6C show scatter plots comparing the levels in peripheral blood (X axis) and cf-mRNA (Y axis) of neutrophil-specific and T-cell-specific transcripts. Arrows point to neutrophil progenitor transcripts and mature transcripts are shown as well. Both x-axis and y-axis show TPM in loge scale. FIGS. 6D-6E show box-plots comparing the normalized levels (TPM) of the indicated hematopoietic progenitor transcripts measured by RNA-Seq in paired buffy coat and cf-mRNA samples (n=5; p-value, t-test). Boxes map median 25th and 75th quintiles, and the whiskers extend to 1.5× interquartile range (IQR). FIG. 6F show levels of BM-specific (left) and whole blood-specific genes (right) were compared in matching plasma and whole blood of 3 individuals. Average fold change (plasma/whole blood) of these transcripts is shown. P value, t test.

FIGS. 7A-7E show cf-mRNA contains Ig transcripts derived from plasma cells in the BM of Multiple Myeloma patients. FIGS. 7A-7C show levels of Ig transcripts measured by RNA-Seq in plasma and buffy coat of a MM patient undergoing BM ablation (starting day −2) and autologous stem cell transplantation (day 0). Bar graphs show the normalized levels (TPM) of Ig heavy chain constant region transcripts (A), light chain constant region transcripts (B) and lambda light chain variable region transcripts (c) detected during the study. Day of blood collection with respect to the time of transplant is indicated in the X axis. Ig transcripts IGHG1 and IGKC dominate the plasma sample, matching the results obtained by molecular testing performed in BM biopsy of this patient (Table 7). FIG. 7D-7E show fraction of Ig heavy and light variable chain transcripts over time in cf-mRNA of MM Patient 1 and Patient 3. Dominant transcripts are shown in solid line 702 and solid line 704. Time with respect to transplant day is shown.

FIGS. 8A-8D show monitoring transcriptional activity of BM hematopoietic lineages by cf-mRNA in Acute Myeloid Leukemia (AML) patients undergoing BM ablation and transplant. FIGS. 8A-8C show time course of normalized levels (TPM) of erythrocyte (A), megakaryocyte (B) and neutrophil (C) specific transcripts in AML Patient 2. Corresponding peripheral blood counts are plotted in the secondary axis of each graph and represented with a black dotted line (RBC count (A), platelet count (B) and neutrophil count (C). Day of blood collection with respect to the time of transplant (day 0) is indicated in the X axis. FIG. 8D shows Time course of mature and immature neutrophil components in AML patients. Neutrophil count is shown in dashed line. Immature transcripts are detected in cf-mRNA days before neutrophil count recovers. Day of blood collection with respect to the time of transplant is indicated in the X axis.

FIGS. 9A-9F show monitoring BM transcriptional activity by cf-mRNA profiling in a Multiple Myeloma patient during BM ablation and transplant. FIGS. 9A and 9B show time course of red blood cell counts (RBC, dashed black line) and hemoglobin transcripts (solid lines) in multiple myeloma Patient 2 during chemotherapy and BM reconstitution (see also FIG. 3). Day of blood collection with respect to the time of transplant is indicated in the X axis. FIGS. 9C-9F show that RNA-Seq was performed in cf-mRNA and matching buffy coat samples. Graphs show the fold change relative to baseline of key erythrocyte (C) and megakaryocyte transcripts (D), as well as mature neutrophil (E) and immature neutrophil-specific transcripts (F) in both specimens. In all panels, black lines represent the relative changes in corresponding circulating cell blood counts: RBC counts (C), platelet counts (D) and neutrophil counts (E, F). Day of blood collection with respect to the time of transplant is indicated in the X axis.

FIGS. 10A-10C show lineage specific-genes in cf-mRNA by growth factors after EPO treatment. FIG. 10A shows fold change over time of key erythrocyte developmental genes (indicated) in EPO treated patients relative to baseline. The general trends show elevated levels of these transcripts after EPO treatment with a return to basal levels at later time points. FIGS. 10B and 10C show fold change of immature (A) and mature (B) neutrophil specific transcripts in cf-mRNA of a patients after treatment with G-CSF. Day 0 (before treatment) is used as reference. Fold change of indicated transcripts is shown for 3 patients, patient 1 represented with dashed line, patient 2 represented with grey solid line, and patient 3 represented with dark solid line. Time points across each Patient are connected by lines. Day of blood collection with respect to the time of treatment is indicated in the X axis.

FIG. 11 shows a computer system that is programmed or otherwise configured to measure and analyze cf-mRNA transcripts described herein in samples.

### DETAILED DESCRIPTION

Biological processes underlying the presence of mRNA transcripts in circulation remain unknown. In the case of cfDNA, studies have shown the mechanism is passive release into circulation upon cell death. In contrast, RNA molecules can be actively secreted from cells. Work has focused on the secretion of non-coding and smaller RNA molecules into exosomes and other lipid vesicles. However, on a per molecule basis, mRNA may comprise a minor fraction of this phenomenon.

Advances in cfDNA technology have resulted in the development of clinically applicable cf-NA-based biomarkers. cfDNA may offer potential advantages compared to invasive tissue biopsies; however, cfDNA analyses can rely on mutations, polymorphisms, or structural variation, which may prevent its use in disease and physiological scenarios not associated with genetic differences. cfDNA methylation analyses have been used as a surrogate of tissue-specific gene expression.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Unless otherwise indicated, open terms, for example, “contain,” “containing,” “include,” “including,” and the like, as used herein, generally mean comprising.

The singular forms “a,” “an,” and “the,” as used herein, generally include plural references unless the context clearly dictates otherwise. Accordingly, unless the contrary is indicated, the numerical parameters set forth in this application are approximations that may vary depending upon the desired properties sought to be obtained by the present invention.

Unless otherwise indicated, some instances herein contemplate numerical ranges. When a numerical range is provided, unless otherwise indicated, the range includes the range endpoints. Unless otherwise indicated, numerical ranges include all values and subranges therein as if explicitly written out. Unless otherwise indicated, any numerical ranges and/or values herein, following or not following the term “about,” can be at 85-115% (i.e., plus or minus 15%) of the numerical ranges and/or values.

The term “subject,” as used herein, generally refers to any individual that is healthy or has, may have, or may be suspected of having a disease condition. The disease condition may include an organ failure, which may require an organ transplant, e.g., bone marrow transplant, liver transplant, lung transplant, heart transplant, face transplant, etc. The subject may be an animal. The animal can be a mammal, such as a human, non-human primate, a rodent such as a mouse or rat, a dog, a cat, pig, sheep, or rabbit. Animals can be fish, reptiles, or others. Animals can be neonatal, infant, adolescent, or adult animals. The subject may be a living organism. The subject may be a human. Humans can be greater than or equal to 1, 2, 5, 10, 20, 30, 40, 50, 60, 65, 70, 75, 80 or more years of age. A human may be from about 18 to about 90 years of age. A human may be from about 18 to about 30 years of age. A human may be from about 30 to about 50 years of age. A human may be from about 50 to about 90 years of age. The subject may be healthy that may need monitoring of the subject's organ status. The subject may have one or more risk factors of a condition and be asymptomatic. The subject may be asymptomatic of a condition. The subject may have one or more risk factors for a condition. The subject may be symptomatic for a condition. The subject may be symptomatic for a condition and have one or more risk factors of the condition. The subject may have or be suspected of having a disease, such as arthritis. The subject may be a patient being treated for a disease, such as arthritis. The subject may be predisposed to a risk of developing a disease such as arthritis. The subject may be in remission from a treatment to the condition. The treatment may include organ transplant.

The term “sample,” as used herein, generally refers to any sample of a subject (such as a blood sample, a urine sample, a sweat sample, a semen sample, a vaginal discharge sample, a cell-free sample, a tissue sample, a tumor biopsy sample, a bone marrow sample, or any other types of biofluids). Genomic data may be obtained from the sample. A blood sample may be a whole blood sample or a peripheral blood sample. A blood sample may be a serum sample. A blood sample may be a plasma sample. Serum and plasma both come from the liquid portion of the whole blood that remains once the cells are removed. Serum is the liquid that remains after the blood has clotted. Plasma is the liquid that remains when clotting is prevented with the addition of an anticoagulant. A blood sample may be a buffy coat sample. The buffy coat is the fraction of an anticoagulated blood sample that contains most of the white blood cells and platelets following density gradient centrifugation of the whole blood sample.

In general, the terms “cell-free polynucleotide,” and “cell-free nucleic acid,” as used interchangeable herein, refer to a polynucleotide that can be isolated from a sample without extracting the polynucleotide from a cell. Cell-free polynucleotides disclosed herein are typically polynucleotides that have been released or secreted from a healthy tissue, damaged tissue, healthy organ, or damaged organ. In some cases, cell-free messenger RNA derived from circulating cells and/or specific tissue/organ residing cells are found in either healthy subject or subject with a condition. For example, damage to the tissue or organ may be due to a disease, injury or other condition that resulted in cytolysis, releasing the cell-free polynucleotide from cells of the damaged tissue into circulation. In some instances, a cell-free polynucleotide disclosed herein is tissue-specific. In other instances, a cell-free polynucleotide is not tissue-specific. In some instances, a cell-free polynucleotide is present in a cell or in contact with a cell. In some instances, a cell-free polynucleotide is in contact with an organelle, vesicle, or exosome. In some instances, a cell-free polynucleotide is cell-free, meaning the cell-free polynucleotide is not in contact with a cell. Cell-free polynucleotides described herein are freely circulating, unless otherwise specified. In some instances, a cell-free polynucleotide is freely circulating, that is the cell-free polynucleotide is not in contact with any vesicle, organelle, or cell. In some instances, a cell-free polynucleotide is associated with a polynucleotide-binding protein (transferases, ribosomal proteins, etc.), but not any other molecules. Understanding the mechanisms underlying the presence of mRNA transcripts in circulation can be used to interpret their clinical value. For example, cfDNA has been shown to originate primarily from dying cells; therefore, the use of this “liquid biopsy” relies on scenarios associated with cell death. Changes in cf-mRNA levels may be influenced by transcriptional changes in living cells during maturation, proliferation and response to stimuli, without requiring cell death.

The term, “marker,” as used herein, generally encompasses a wide variety of biological molecules. Markers may also be referred to herein as disease markers, markers of disease, or markers indicating a status of an organ (e.g., whether the organ is functionally proper after transplanting). In some instances, the marker is for a condition associated with a plurality of diseases. For example, the marker may be for inflammation, which can be associated with cancer or transplanted organ failure. Markers, by way of non-limiting example, include peptides, hormones, lipids, vitamins, pathogens, cell fragments, metabolites, and nucleic acids. In some instances, a marker is a cell-free nucleic acid. In some cases, markers disclosed herein are not tissue-specific. However, in some instances, the markers are tissue-specific. Markers disclosed herein may also be referred to as disease and/or condition biomarkers. The disease biomarker is a biological molecule that is present or produced as a result of a disease and/or condition, dysregulated as a result of a disease and/or condition, mechanistically implicated in a disease and/or condition, mutated or modified in a disease and/or condition state, or any combination thereof. Markers may be produced by the subject. Markers may also be produced by other species. For instance, the marker may be a nucleic acid or protein made by a hepatitis virus or a Streptococcus bacterium. Methods identifying such markers may further comprise detecting and/or quantifying tissue-specific polynucleotides to determine which tissues are infected or affected by these pathogens, and optionally, to an extent that the tissue(s) are damaged. Markers of diseases disclosed herein generally do not circulate in individuals unaffected by the disease.

The term “sequencing” as used herein, may comprise sequencing by synthesis, high-throughput sequencing, next-generation sequencing, Maxam-Gilbert sequencing, massively parallel signature sequencing, Polony sequencing, 454 pyrosequencing, pH sequencing, Sanger sequencing (chain termination), Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, Enigma sequencing, sequencing-by-hybridization, sequencing-by-ligation, or any combination thereof. The sequencing output data may be subject to quality controls, including filtering for quality (e.g., confidence) of base reads. Exemplary sequencing systems include 454 pyrosequencing (454 Life Sciences), Illumina (Solexa) sequencing, SOLiD (Applied Biosystems), and Ion Torrent Systems' pH sequencing system. In some cases, a nucleic acid of a sample may be sequenced without an associated label or tag. In some cases, a nucleic acid of a sample may be sequenced, the nucleic acid of which may have a label or tag associated with it.

Disclosed herein are methods, systems, databases, and compositions related to using tissue and/or organ specific cell-free mRNA (cf-mRNA) transcripts to monitor a healthy subject's organ status or a subject having a condition and/or disease's organ status. Further, the tissue and/or organ specific cell-free mRNA (cf-mRNA) transcripts may also be used to monitor a subject's organ after the subject received a treatment directed to the organ. Cf-mRNA transcriptome can be considered as a compendium of transcripts collected from all organs. Since some of these circulating transcripts correspond to well-characterized tissue-specific genes, they can be used to monitor the health or state of individual tissues of origin. Indeed, cf-mRNA may also be used to reflect fetal development, predict preterm delivery in pregnant women, and as a cancer biomarker.

As described herein, a proof of concept study was conducted. The current disclosure provides proof of concept of using cf-mRNA profiling to monitor bone marrow (BM) activity, which could lead to improved therapeutic management of patients with BM disease, and alleviate the need for invasive BM biopsies. For example, next-generation sequencing (NGS)-based whole-transcriptomic profiling of cf-mRNA was conducted. Expression levels of cf-mRNA were compared to those from circulating cells of the blood (CC) to decipher the origin of circulating transcripts and better understand their potential clinical utility. Most cf-mRNA transcripts may be of hematopoietic origin. In both healthy subjects and multiple myeloma patients, cf-mRNA can be enriched in BM-specific transcripts. Further, longitudinal studies of cancer patients undergoing BM ablation and transplantation showed that cf-mRNA profiling can non-invasively capture temporal transcriptional activity of the BM. Mechanistically, stimulation of specific BM-lineages with growth factor therapeutics indicates that cf-mRNA fluctuations reflect active lineage-specific transcriptional activity. Collectively, the present disclosure provides insights into the biological origins of cf-mRNA, indicating that living cells may secrete cf-mRNA.

Further, cf-mRNA profiling can provide broader molecular information compared to other non-invasive biomarkers and can constitutes a non-invasive approach to examine tissue function in scenarios such as monitoring of diseases and drug response in subjects. For example, melphalan-induced apoptosis did not significantly increase the levels of cf-mRNA. In contrast, a large increase of transcripts in circulation was observed during BM reconstitution and upon stimulation with well-known pro-survival and antiapoptotic growth factors. In vitro studies have shown that extracellular mRNA levels and composition can change upon cellular stimulation and that living cells can secrete RNA molecules embedded in vesicles. Additionally, the present disclosure demonstrates that the circulating transcriptome can be a dynamic entity that allows constant measurement of tissue function over time. This is in contrast to cfDNA methylation and mutation events, which can be less dynamic and may provide limited information on tissue homeostasis.

### Monitoring a Subject's Healthy State

The cf-mRNA transcriptome can provide direct access to both genetic information as well as information pertaining to the tissue of origin and its physiology. For instance, the genetic alterations in cf-mRNA can provide information for monitoring allografts, and similar approaches can diagnose fetal chromosomal abnormalities. Given that tumor derived transcripts in circulation have been identified, the genetic information captured by cf-mRNA can be of interest in cancer diagnosis and monitoring. In addition, cf-mRNA can provide tissue-specific transcripts that reveal functional information pertaining the tissue of origin. The cf-mRNA can capture transcripts that may reveal BM physiology in both healthy subjects and cancer patients. Therefore, cf-mRNA may integrate functional and genetic information of tissues.

Another aspect of non-invasive approaches may be that by eliminating the need for surgical tissue acquisition, non-invasive approached may enable repeated assessment of a patient's disease state over time. This can be of significance in several clinical settings, such as monitoring of treatment in cancer patients, where biopsy of affected tissue may remain the gold standard. In this regard, the longitudinal cf-mRNA profiling data discussed herein can show that circulating transcripts capture snapshots of gene expression profiles in tissues such as BM. This can allow non-invasive temporal delineation of BM ablation efficiency, early detection of transplant engraftment, and monitoring of BM reconstitution. For example, in multiple myeloma (MM) patients, cf-mRNA profiling can integrate temporal measurement of clonal Ig transcripts generated by malignant plasma cells in the BM, with detailed BM-lineage transcriptional activity and establishment of a new immune profile. The comprehensive picture revealed by cf-mRNA profiling can provide additional relevant information compared to other non-invasive tests commonly used in this malignancy, such as clonal antibody detection in serum of MM patients. Indeed, given the generally challenging and subjective quantification and characterization of these antibodies, BM biopsies remain as a common practice in the therapy management of MM patients. In addition, unlike antibody detection, cf-mRNA profiling play a role in early identification of suboptimal BM reconstitution, as shown by the lack of development of megakaryocyte lineage in AML Patient 2 as discussed herein.

In some cases, disclosed herein are methods and systems for monitoring a healthy state of a subject's bone marrow, comprising: obtaining a biological sample from the subject having the healthy state; and detecting cell-free mRNA (cf-mRNA) levels of a first plurality of cf-mRNAs derived from the subject's bone marrow and derived cells thereof corresponding to a first plurality of genes. The first plurality of genes may comprise one or more genes from Table 7. For example, cf-mRNA levels of a panel of genes comprising at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, and 370 genes from Table 7 may be used to monitor the healthy state of the subject's BM. Moreover, cf-mRNA levels of a panel of genes comprising up to 377, 365, 355, 345, 335, 325, 315, 305, 295, 285, 275, 265, 255, 245, 235, 225, 215, 205, 195, 185, 175, 165, 155, 145, 135, 125, 115, 105, 95, 85, 75, 65, 55, 45, 35, 25, 15, and 5 genes from Table 7 may be used to monitor the healthy state of the subject's BM.

In addition, the first plurality of genes may comprise genes specific for hematopoietic cells from Table 9. The plurality of genes may comprise erythrocyte-specific genes such as, but not limited to, GATA1, SLC4A1, TF, AVP, RUNDC3A, SOX6, TSPO2, HBZ, TMCC2, SELENBP1, ALAS2, EPB42, GYPA, C17orf99, HBA2, RHCE, HBG2, TRIM10, HBA1, HBM, HBG1, UCA1, GYPB, CTD-3154N5.2, and AC104389.1 The plurality of genes may comprise megakaryocyte-specific genes such as, but not limited to, ITGA2B, RAB27B, GUCY1B3, GP6, HGD, PF4, CLEC1B, CMTM5, GP9, SELP, DNM3, LY6G6F, LY6G6D, XXbac-BPG3213.19, and RP11-879F14.2. The plurality of genes may comprise T-cell-specific genes as listed in Table 9. The plurality of genes may comprise neutrophil-specific genes as listed in Table 9. The plurality of genes may comprise progenitor and/or immature neutrophil-specific genes such as, but not limited to, CTSG, ELANE, AZU1, PRTN3, MMP8, RNASE, and PGLYRP1. Cf-mRNA levels of a panel of genes comprising at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, and 200 genes from Table 9 may be used to monitor the healthy state of the subject's BM. Moreover, cf-mRNA levels of a panel of genes comprising up to 205, 195, 185, 175, 165, 155, 145, 135, 125, 115, 105, 95, 85, 75, 65, 55, 45, 35, 25, 15, and 5 genes from Table 9 may be used to monitor the healthy state of the subject's BM.

In other cases, disclosed here are methods and systems for monitoring a healthy state of a subject's tissue or organ. The methods may comprise obtaining a biological sample from the subject and detecting levels cf-mRNAs correspondingly derived from the tissue or organ. The tissue or organ derived cf-mRNAs can correspond to genes that are specific to the tissue or organ. For example, the tissue may be skin, skeletal muscle, adipose tissue, etc. The organ may be liver, pancreas, lung, heart, brain, etc.

Monitoring a Subject's Organ with a State of a Condition and/or Disease

In some cases, disclosed here are methods and systems for monitoring a disease state of a subject's bone marrow, comprising obtaining a biological sample from the subject having the disease state; and detecting cell-free mRNA (cf-mRNA) levels of a second plurality of cf-mRNAs derived from a plurality of cells resident or originated from the bone marrow corresponding to a second plurality of genes.

In some cases, the organ is bone marrow. The cf-mRNAs detected from a biological sample, such as a blood sample, may correspond to genes specific to bone marrow with a particular condition or disease. In some cases, the condition may be anemia. Anemia can be a common blood disorder, and according to the National Heart, Lung, and Blood Institute, anemia affects more than 3 million Americans. Red blood cells can carry hemoglobin, an iron-rich protein that attaches to oxygen in the lungs and carries it to tissues throughout the body. Anemia can occur when a subject does not have enough red blood cells or when the subject's red blood cells do not function properly. Anemia can be diagnosed when a blood test shows a hemoglobin value of less than 13.5 gm/dl in a man or less than 12.0 gm/dl in a woman. Monitoring the levels of cf-mRNA corresponding to erythrocyte-specific genes from Table 9 may be more transient and dynamic than counting cell count of erythrocytes in the peripheral blood sample.

In some cases, the disease may be multiple myeloma (MM). Multiple myeloma is a blood cancer that can be related to lymphoma and leukemia. In multiple myeloma, a type of white blood cell called a plasma cell generally multiplies unusually. Normally, the plasma cells may make antibodies that fight infections. But in multiple myeloma, the plasma cells can release too much protein (called immunoglobulin) into a subject's bones and blood. Immunoglobulin can build up throughout the subject's body and cause organ damage. A plurality of genes may be associated with MM, such as, but not limited to, IGHG1, IGHA1, IGKC, IGHV1, IGHV2, IGHV3, IGHV4, IGHV5, IGHV6, IGHV7, IGHV8, IGHV9, IGHV10, IGHV11, IGHV12, IGHV13, IGHV14, IGHV15, IGHV16, IGHV17, IGHV18, IGHV19, IGHV20, IGHV21, IGHV22, IGHV23, IGHV24, IGHV25, IGHV26, IGHV27, IGHV28, IGHV29, IGHV30, IGHV31, IGHV32, IGHV33, IGHV34, IGHV35, IGHV36, IGHV37, IGHV38, IGHV39, IGHV40, IGHV41, IGHV42, IGHV43, IGHV44, IGHV45, IGHV46, IGHV47, IGHV48, IGHV49, IGHV50, IGHV51, IGHV52, IGHV53, IGHV54, IGHV55, IGHV56, IGHV57, IGHV58, IGHV59, IGHV60, IGHV61, IGHV62, IGHV63, IGHV64, IGHV65, IGHV66, IGHV67, IGHV68, IGHV69, IGKV2, IGKV3, IGKV4, IGKV5, IGKV6, IGKV7, IGKV8, IGKV9, IGKV10, IGKV11, IGKV12, IGKV13, IGKV14, IGKV15, IGKV16, IGKV17, IGKV18, IGKV19, IGKV20, IGKV21, IGKV22, IGKV23, IGKV24, IGL1, and IGLV 1-40. By detecting levels of cf-mRNAs corresponding to those genes associated with MM from a blood sample, the need to obtain BM biopsy to monitor the MM prognosis may be alleviated.

Further, in some case, the disease may be lymphoma, leukemia, myeloproliferative neoplasms, or myelodysplastic syndrome. Lymphoma is cancer that can begin in infection-fighting cells of the immune system, called lymphocytes. Lymphocytes can be in the lymph nodes, spleen, thymus, bone marrow, and other parts of the body. When one has lymphoma, lymphocytes change and can grow out of control. By detecting levels of cf-mRNAs corresponding to genes specifically associated with or tied to lymphoma from a blood sample, the need of obtaining a BM biopsy may be removed.

Leukemia can be a cancer of the early blood-forming cells. Generally, leukemia is a cancer of the white blood cells, but some leukemias can start in other blood cell types. There are several types of leukemia, which can be divided based on whether the leukemia is acute (fast growing) or chronic (slower growing), and whether the leukemia starts in myeloid cells or lymphoid cells. By detecting levels of cf-mRNAs corresponding to genes specifically associated with or tied to different types of leukemia from a blood sample, the need of obtaining a BM biopsy may be removed.

Myeloproliferative neoplasms (MPNs) can be blood cancers that occur when the body makes too many white or red blood cells, or platelets. This overproduction of blood cells in the bone marrow can create problems for blood flow and lead to various symptoms. By detecting levels of cf-mRNAs corresponding to genes specifically associate with or tied to MPNs from a blood sample, the need of obtaining a BM biopsy may be removed.

Further, myelodysplastic syndromes (MDS) are a group of cancers in which immature blood cells in the bone marrow may not mature and therefore do not become healthy blood cells. Early on, there are generally no symptoms. Later symptoms may include feeling tired, shortness of breath, easy bleeding, or frequent infections. By detecting levels of cf-mRNAs corresponding to genes specifically associated with or tied to MDS from a blood sample, the need of obtaining a BM biopsy may be removed. Myelofibrosis is an uncommon type of bone marrow cancer that disrupts your body's normal production of blood cells. Myelofibrosis causes extensive scarring in your bone marrow, leading to severe anemia that can cause weakness and fatigue. By detecting levels of cf-mRNAs corresponding to genes specifically associated with or tied to myelofibrosis from a blood sample, the need of obtaining a BM biopsy may be removed. Polycythemia vera is a slow-growing blood cancer in which your bone marrow makes too many red blood cells. These excess cells thicken your blood, slowing its flow. They also cause complications, such as blood clots, which can lead to a heart attack or stroke. By detecting levels of cf-mRNAs corresponding to genes specifically associated with or tied to myelofibrosis from a blood sample, the need of obtaining a polycythemia vera biopsy may be removed.

In addition, thrombocythemia is a disease in which your bone marrow makes too many platelets. Platelets are blood cell fragments that help with blood clotting. Having too many platelets makes it hard for your blood to clot normally. This can cause too much clotting, or not enough clotting. By detecting levels of cf-mRNAs corresponding to genes specifically associated with or tied to thrombocythemia from a blood sample, the need of obtaining a BM biopsy may be removed.

Moreover, bone marrow specific cell free polynucleotides can be used to monitor a compound/therapies listed herein in treating a bone marrow disease. For example, certain bone marrow specific cell free polynucleotides (e.g. cf-mRNAs as disclosed herein) can be used to assess effectiveness of a ubiquitin ligase inhibitor (e.g., iberdomide that specifically target the cereblon E3 ligase enzyme) in treating MM at various time points without any invasive procedures. A blood sample can be drawn from a subject before receiving iberdomide at a first time point to assess bone marrow specific cf-mRNAs at the first time point. Subsequently, various blood samples can be obtained at various time points, such as 2 days after treating the subject with iberdomide, 4 days after such treatment, 8 days afterwards, 16 days afterwards, 30 days afterwards, 60 days afterwards, 120 days afterwards, 4 months afterwards, 6 months afterwards, 12 months afterwards, 18 months afterwards, 24 months afterwards, 36 months afterwards, 48 months afterwards, to assess bone marrow specific cf-mRNAs at these various time points respectively. The different length of days and/or months after the treatment begin listed here is not meant to be limiting. A researcher/medical worker can choose different time points based on different compounds, therapies, diseases to be treated, and other parameters.

In some cases, disclosed herein are methods and systems for monitoring a disease state of a subject's organ, such as liver, heart, central nervous system, etc. For example, when a subject is suffering from non-alcoholic fatty liver disease disorder (NAFLD), which may require constant monitoring by a healthy care provider. By detecting liver specific cf-mRNAs from a blood sample provides a convenient and non-invasive method in monitoring NAFLD condition. Liver specific cf-mRNAs corresponding to various liver specific genes may also be used to monitor effectiveness of a compound/therapy in treating NAFLD.

For various conditions and diseases associated with a subject's heart and cardiovascular system, heart specific cf-mRNAs from a blood sample provides a convenient and non-invasive method in monitoring any cardiovascular conditions and diseases. Further, heart specific cf-mRNAs corresponding to various heart specific genes may also be used to monitor effectiveness of a compound/therapy in treating a specific cardiovascular condition.

With respect to any central nervous system (CNS) conditions or diseases, CNS specific cf-mRNAs may be used to provide a convenient and non-invasive method in monitoring any CNS conditions and diseases. Moreover, CNS specific cf-mRNAs corresponding to various CNS conditions and diseases may be used to monitor effectiveness of a compound/therapy in treating a specific cardiovascular condition.

### Monitoring a Treatment State of a Subject's Organ

In some cases, disclosed herein are methods and systems for monitoring a treatment state of a subject's organ, comprising obtaining a plasma sample from the subject having the treatment state; and detecting cell-free mRNA (cf-mRNA) levels of a third plurality of cf-mRNAs derived from the subject's organ corresponding to a second plurality of genes. In some cases, the organ is bone marrow. In some cases, the treatment of a bone marrow condition or disease comprises bone marrow ablation, bone marrow reconstitution, bone marrow transplant, stimulation with growth factors, immunotherapy, immunomodulation, modulation of the activity of ubiquitin ligases, or autologous or heterologous CAR-T cell therapy.

Bone marrow ablation is generally performed before bone marrow reconstitution and bone marrow transplant to treat blood conditions and diseases. The bone marrow ablation may comprise physical ablation, such as ionizing irradiation; or chemical ablation, such as melphalan-mediated bone marrow ablation, busulfan-mediated bone marrow ablation, treosulfan-mediated ablation, chemotherapy-mediated ablation, etc. Utilizing the methods provided herein, whether the bone marrow ablation procedure is performed successfully can be monitored in a quick and non-invasive manner by measuring cf-mRNAs levels corresponding to erythrocyte-specific genes, neutrophil-specific genes, progenitor-neutrophil-specific genes, T-cell-specific genes, and/or other genes that can be used to indicate the original diseased bone marrow has been ablated from a blood sample. In some cases, the erythrocyte-specific genes may comprise one or more genes from the group including, but not limited to, GATA1, SLC4A1, TF, AVP, RUNDC3A, SOX6, TSPO2, HBZ, TMCC2, SELENBP1, ALAS2, EPB42, GYPA, C17orf99, HBA2, RHCE, HBG2, TRIM10, HBA1, HBM, HBG1, UCA1, GYPB, CTD-3154N5.2, and AC104389.1 as listed in Table 9. In some cases, the neutrophil-specific genes may comprise one or more genes from Table 9 listed in the column of neutrophil. In some cases, the progenitor-neutrophil-specific genes may comprise one or more genes from the group including, but not limited to, CTSG, ELANE, AZU1, PRTN3, MMP8, RNASE, and PGLYRP1 as listed in Table 9. In some cases, the T-cell-specific genes may comprise one or more genes from Table 9 in the column of T-cells.

After bone marrow ablation, bone marrow reconstitution, allogenic bone marrow transplant, or autologous bone marrow transplant may be performed to replenish the subject suffering from a blood disease with healthy hematopoietic stem cells, which can develop into erythrocytes, white blood cells, neutrophils, eosinophils, basophils, lymphocytes, and monocytes in regulating immune responses. The methods disclosed herein may be used to monitor cf-mRNA levels corresponding to the different cell-type specific genes from a blood sample to determine whether BM reconstitution or transplant procedure is successful. Further, measurement (e.g., repeated measurement) of the cf-mRNA levels may be used to monitor the subject's prognosis after the treatment of BM reconstitution or transplant. For example, cf-mRNAs levels corresponding to erythrocyte-specific genes, megakaryocyte-specific genes, neutrophil-specific genes, progenitor-neutrophil-specific genes, T-cell-specific genes, or other suitable cell-type-specific genes may be measured. In some cases, the megakaryocyte-specific genes may comprise one or more genes from the group of genes including, but not limited to, ITGA2B, RAB27B, GUCY1B3, GP6, HGD, PF4, CLEC1B, CMTM5, GP9, SELP, DNM3, LY6G6F, LY6G6D, XXbac-BPG3213.19, and RP11-879F14.2 as listed in Table 9. In some cases, the erythrocyte-specific genes may comprise one or more genes from the group including, but not limited to, GATA1, SLC4A1, TF, AVP, RUNDC3A, SOX6, TSPO2, HBZ, TMCC2, SELENBP1, ALAS2, EPB42, GYPA, C17orf99, HBA2, RHCE, HBG2, TRIM10, HBA1, HBM, HBG1, UCA1, GYPB, CTD-3154N5.2, and AC104389.1 as listed in Table 9. In some cases, the neutrophil-specific genes may comprise one or more genes from Table 9 listed in the column of neutrophil. In some cases, the progenitor-neutrophil-specific genes may comprise, but are not limited to, CTSG, ELANE, AZU1, PRTN3, MMP8, RNASE, and PGLYRP1 as listed in Table 9. In some cases, the T-cell-specific genes may comprise one or more genes from Table 9 in the column of T-cells.

Immunotherapy and immunomodulation treatments can be used to boost a subject's immune system to treat cancer, such as MM, leukemia, lymphoma, etc. Types of immunotherapy include, but are not limited to, administering monoclonal antibodies, immune checkpoint inhibitors, or cancer vaccinations to the subject in need thereof. Chimeric antigen receptor (CAR) T-cell therapy can be another type of immunotherapy. Generally, for autologous CAR-T therapy, T cells can be collected via apheresis from a subject, a procedure during which blood may be withdrawn from the body and one or more blood components (such as plasma, platelets, or white blood cells) may be removed. Subsequently, the T cells can be sent to a laboratory or a drug manufacturing facility where they are genetically engineered, e.g., by introducing DNA into them, to produce chimeric antigen receptors (CARs) on the surface of the cells. CARs are proteins that can allow the T cells to recognize an antigen on targeted tumor cells. The number of the subject's genetically modified T cells can be “expanded” by growing cells in the laboratory. When there are sufficient cells, these CAR T cells may be frozen and/or infused into the subject.

During immunotherapy and/or immunomodulation treatment, cf-mRNAs levels corresponding to erythrocyte-specific genes, megakaryocyte-specific genes, neutrophil-specific genes, progenitor-neutrophil-specific genes, T-cell-specific genes, or other suitable cell-type-specific genes may be utilized to monitor the effectiveness of the treatment. Based on the transient and/or non-invasive measurement, different types of immunotherapy and/or immunomodulation with different doses can be adjusted to achieve a desired response in a subject. In some cases, the megakaryocyte-specific genes comprise one or more genes from the group of genes including, but not limited to, ITGA2B, RAB27B, GUCY1B3, GP6, HGD, PF4, CLEC1B, CMTM5, GP9, SELP, DNM3, LY6G6F, LY6G6D, XXbac-BPG3213.19, AND RP11-879F14.2 as listed in Table 9. In some cases, the erythrocyte-specific genes may comprise one or more genes from the group including, but not limited to, GATA1, SLC4A1, TF, AVP, RUNDC3A, SOX6, TSPO2, HBZ, TMCC2, SELENBP1, ALAS2, EPB42, GYPA, C17orf99, HBA2, RHCE, HBG2, TRIM10, HBA1, HBM, HBG1, UCA1, GYPB, CTD-3154N5.2, and AC104389.1 as listed in Table 9. In some cases, the neutrophil-specific genes may comprise one or more genes from Table 9 listed in the column of neutrophil. In some cases, the progenitor-neutrophil-specific genes may comprise, but are not limited to CTSG, ELANE, AZU1, PRTN3, MMP8, RNASE, and PGLYRP1 as listed in Table 9. In some cases, the T-cell-specific genes may comprise one or more genes from Table 9 in the column of T-cells.

Further, for growth factor stimulation treatment, such as erythropoietin (EPO) and granulocyte colony stimulating factor (G-CSF), cf-mRNAs levels corresponding to erythrocyte-specific genes, megakaryocyte-specific genes, neutrophil-specific genes, progenitor-neutrophil-specific genes, T-cell-specific genes, or other suitable cell type-specific genes may be utilized to monitor the effectiveness of the treatment. Based on the transient and/or non-invasive measurement, different doses and/or regimes of the growth factors may be used achieve a desired response in a subject. In some cases, the megakaryocyte-specific genes can comprise one or more genes from the group of genes including, but not limited to, ITGA2B, RAB27B, GUCY1B3, GP6, HGD, PF4, CLEC1B, CMTM5, GP9, SELP, DNM3, LY6G6F, LY6G6D, XXbac-BPG3213.19, AND RP11-879F14.2 as listed in Table 9. In some cases, the erythrocyte-specific genes may comprise one or more genes from the group including, but not limited to, GATA1, SLC4A1, TF, AVP, RUNDC3A, SOX6, TSPO2, HBZ, TMCC2, SELENBP1, ALAS2, EPB42, GYPA, C17orf99, HBA2, RHCE, HBG2, TRIM10, HBA1, HBM, HBG1, UCA1, GYPB, CTD-3154N5.2, and AC104389.1 as listed in Table 9. In some cases, the neutrophil-specific genes may comprise one or more genes from Table 9 listed in the column of neutrophil. In some cases, the progenitor-neutrophil-specific genes may comprise, but are not limited to, CTSG, ELANE, AZU1, PRTN3, MMP8, RNASE, and PGLYRP1 as listed in Table 9. In some cases, the T-cell-specific genes may comprise one or more genes from Table 9 in the column of T-cells.

### Isolating, Quantifying, and Detecting

Some methods disclosed herein comprise isolating at least one tissue-specific polynucleotide. In some cases, the at least one tissue-specific polynucleotide comprise a cell-free polynucleotide. In some cases, isolating the cell-free polynucleotide may comprise fractionating the sample from the subject. Some methods may comprise removing intact cells from the sample. For example, some methods may comprise centrifuging a blood sample and collecting the supernatant that is serum or plasma, or filtering the sample to remove cells. In some embodiments, cell-free polynucleotides may be analyzed without fractionating the sample from the subject. For example, urine, cerebrospinal fluid, or other fluids that contain little to no cells may not require fractionating. Some methods may comprise sufficiently purifying the cell-free polynucleotides in order to detect, quantify, and/or analyze the cell-free polynucleotides. Various reagents, methods, and kits can be used to purify the cell-free polynucleotides. Reagents may include, but are not limited to, phenol, detergents, chaotropic salts, Trizol, phenol-chloroform, glycogen, sodium iodide, and guanidine resin, affinity columns, desalting columns Kits include, but are not limited to, Thermo Fisher ChargeSwitch® Serum Kit, Qiagen RNeasy Kit, ZR serum DNA kit, Puregene DNA purification system, QIAamp DNA Blood Midi kit, QIAamp Circulating Nucleic Acid Kit, and QIAamp DNA Mini kit.

Some methods disclosed herein can comprise enriching a sample for cell-free polynucleotides. For example, a sample of interest may contain RNA and/or DNA from bacteria. Some methods may comprise exomal capture, thereby eliminating, or substantially eliminating, unwanted sequences and enriching the sample for polynucleotides of interest. In some cases, exomal capture comprises array-based capture or in-solution capture, fragments of DNA corresponding to RNAs of interest tethered to a surface or beads, respectively. Some methods also comprise filtering or removing other biological molecules or cells from the sample, such as proteins or platelets. In some instances, enriching the sample for cell-free polynucleotides includes preventing blood cell RNA contamination of a plasma sample. In some instances, using tubes free of EDTA may prevent or reduce the presence of blood cell RNA in a plasma and/or serum sample.

Generally, methods disclosed herein may comprise detecting or quantifying at least one tissue-specific polynucleotide. In some instances, quantifying and/or detecting the at least one tissue-specific polynucleotide may comprise amplifying the at least one tissue-specific polynucleotide. In some cases involving cell-free RNA, quantifying and/or detecting the at least one tissue-specific polynucleotide may comprise reverse transcribing the cell-free RNA. Any of a variety of processes can be employed to detect and/or quantify the marker or tissue-specific polynucleotide in a sample. In some cases involving cell-free, tissue-specific RNAs, RNA may be isolated from a sample and reverse transcribed to produce cDNA prior to further manipulation, such as amplification and/or sequencing. In some embodiments, amplification may be initiated at the 3′ end as well as randomly throughout the whole transcriptome in the sample to allow for amplification of both mRNA and non-polyadenylated transcripts. Suitable kits for amplifying cDNA include, for example, the Ovation® RNA-Seq System. Tissue-specific RNAs can be identified and quantified by a variety of techniques such as, but not limited to, array hybridization, quantitative PCR, and sequencing.

Some methods of quantifying nucleic acids disclosed herein may comprise measuring at least one nucleic acid. Measurement can be done by sequencing. Sequencing may be targeted sequencing. In some cases, targeted sequencing can comprise specifically amplifying a select marker or a select tissue-specific polynucleotide as disclosed herein and sequencing the amplification products. In some cases, targeted sequencing can comprise specifically amplifying a subset of selected markers or a subset of select tissue-specific polynucleotides as disclosed herein and sequencing the amplification products. Alternatively, some methods comprising targeted sequencing may not comprise amplifying the markers or tissue-specific polynucleotides. Some methods may comprise untargeted sequencing. In some instances, untargeted sequencing can comprise sequencing the amplification products, a portion of the cell-free nucleic acids are not markers or tissue-specific polynucleotides. In some instances, untargeted sequencing may comprise amplifying cell-free nucleic acids in a sample from the subject and sequencing the amplification products, a portion of the cell-free nucleic acids are not markers or tissue-specific polynucleotides. In some instances, untargeted sequencing can comprise amplifying cell-free nucleic acids comprising a marker or tissue-specific polynucleotide described herein. Sequencing may provide a number of reads that corresponds to a relative quantity of the marker or tissue-specific polynucleotide. In some instances, sequencing may provide a number of reads that corresponds to an absolute quantity of the marker or tissue-specific polynucleotide. In some embodiments, the amplified cDNA may be sequenced by whole transcriptome shotgun sequencing (also referred to as “RNA-Seq”). Whole transcriptome shotgun sequencing (RNA-Seq) can be accomplished using a variety of next-generation sequencing platforms such as, but not limited to, the Illumina Genome Analyzer platform, ABI Solid Sequencing platform, or Life Science's 454 Sequencing platform. In some instances, identification of specific targets may be performed by microarray, such as a peptide array or oligonucleotide array, in which an array of addressable binding elements specifically bind to corresponding targets, and a signal proportional to the degree of binding is used to determine quantity of the target in the sample. In some cases, sequencing may be a preferable method of quantifying. In some instances, sequencing can allow for parallel interrogation of thousands of genes without amplicon interference. In some instances, quantifying by sequencing may be preferable to quantifying by Q-PCR. In some instances, there may be so many control genes required to accurately quantify gene expression by Q-PCR, that quantifying with Q-PCR may be inefficient. In other instances, sequencing efficiency and accurate quantification by sequencing may not be affected by the number of (control) genes analyzed. For at least the foregoing reasons, sequencing may be particularly useful for some methods disclosed herein, when the health status of multiple organs (e.g., heart, kidney, and liver) is assessed.

Some methods of quantifying a nucleic acid disclosed herein can comprise quantitative PCR (q-PCR). In some instances, Q-PCR may comprise a reverse transcription reaction of cell-free RNAs described herein to produce corresponding cDNAs. In some instances, cell-free RNA may comprise a marker, a tissue-specific polynucleotide, and a cell-free RNA that is neither a marker nor a tissue specific polynucleotide. Some cell-free RNA comprises a marker described herein, a tissue-specific polynucleotide described herein, and/or a cell-free RNA that is neither a marker nor a tissue specific polynucleotide described herein. In some cases, Q-PCR can comprise contacting the cDNAs that correspond to a marker, a tissue-specific polynucleotide, or a housekeeping gene (e.g., ACTB, ALB, GAPDH, etc.) with PCR primers specific to the marker, tissue-specific polynucleotide, or housekeeping gene.

Some methods disclosed herein comprise quantifying a blood cell-specific polynucleotide. Methods comprising Q-PCR disclosed herein may comprise contacting polynucleotides (either RNA or DNA) with primers corresponding to a tissue-specific polynucleotide. Some hematopoietic cell-specific polynucleotides disclosed herein may be nucleic acids that are predominantly expressed or even exclusively expressed by one or more types of cells. Types of blood cells can be generally categorized as white blood cells (also referred to as leukocytes), red blood cells (also referred to as erythrocytes), and platelets. In some instances, the blood cell-specific polynucleotide may be used as a control in methods comprising quantifying tissue-specific polynucleotides and disease markers disclosed herein. In some cases, absence of an amplification product with primers corresponding to a blood cell-specific polynucleotide may be used to confirm the method is detecting cell-free RNAs in a blood, plasma, or serum sample and not RNA expressed in blood cells. By way of non-limiting example, blood-cell specific polynucleotides can include polynucleotides expressed in white blood cells, platelets, or red blood cells, and combinations thereof. White blood cells include, but are not limited to, lymphocytes, T-cells, B cells, dendritic cells, granulocytes, monocytes, and macrophages. By way of non-limiting example, the bone marrow-specific polynucleotide may be encoded by a gene selected from Table 7.

In some cases, Q-PCR may be a preferable method of quantifying. Q-PCR may be a more sensitive method and therefore may more accurately quantify RNA present at very low levels. In some instances, quantifying by Q-PCR may be preferable to quantifying by sequencing. In some instances, sequencing may require more complex preparation of RNA samples and require depletion or enrichment of nucleic acids in order to provide accurate quantification.

Presence and/or quantity (relative or absolute) of a polynucleotide, as well as changes in sequence resulting from bisulfate treatment, can be detected using any suitable sequence detection method disclosed herein. Examples include, but are not limited to, probe hybridization, primer-directed amplification, and sequencing. Polynucleotides may be sequenced using any suitable low or high throughput sequencing technique or platform, including, but not limited to, Sanger sequencing, Solexa-Illumina sequencing, Ligation-based sequencing (SOLiD), pyrosequencing; strobe sequencing (SMR); and semiconductor array sequencing (Ion Torrent). The Illumina or Solexa sequencing is based on reversible dye-terminators. DNA molecules are generally attached to primers on a slide and amplified so that local clonal colonies are formed. Subsequently, one type of nucleotide at a time may be added, and non-incorporated nucleotides are washed away. Subsequently, images of the fluorescently labeled nucleotides may be taken and the dye is chemically removed from the DNA, allowing a next cycle. The Applied Biosystems' SOLiD technology employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences generally results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. Another example of an envisaged sequencing method is pyrosequencing, in particular 454 pyrosequencing, e.g., based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed, and the cycle is repeated. Further examples of suitable sequencing techniques are sequencing by hybridization, sequencing by use of nanopores, microscopy-based sequencing techniques, microfluidic Sanger sequencing, or microchip-based sequencing methods. High-throughput sequencing platforms can permit generation of multiple different sequencing reads in a single reaction vessel, such as 10³, 10⁴, 10⁵, 10⁶, 10⁷, or more.

### Cell Free Expression Profile

The cell free expression profile comprising a plurality of differentially expressed genes described herein facilitates a sensitive and non-intrusive testing to monitor a treatment (e.g., a pharmaceutical compound)'s effectiveness, measure pharmacodynamics for one or more targets of interest for therapy, measure pharmacodynamics for a lead optimization during drug discovery and development, or monitor a clinical development during therapy. Cell free expression profile comprising a plurality of differentially expressed protein encoding genes are often readily obtained by a blood draw from an individual. Benefits of using the cell free expression profile disclosed herein include fast and convenient monitoring and measuring without cumbersome and unreliable testing.

Various genes can be selected to be included in the cell free expression profile based on higher predictive value than a predicative value of a single gene. Selected genes in the cell free expression profile do not generally co-vary with one another, such that each selected gene provide independent contributions to the cell free expression profile's overall health signatures.

In some cases, various cell free expression profiles, each including a group of different selected genes, for different monitoring or measuring function vary independently from each other. Each cell free expression profile could comprise at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 300, and 400 different genes disclosed herein. Some cell free expression profile including a particular group of selected genes may be used to detect whether a developing drug candidate is effective in treating the disease that is designed to treat.

### Computer Systems

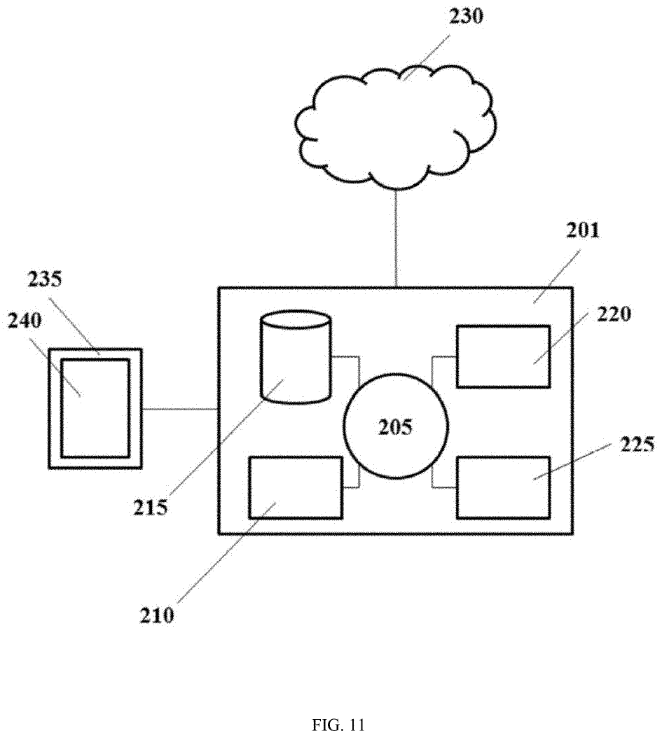

The present disclosure provides computer systems that are programmed to implement methods of the disclosure. FIG. 11 shows a computer system 201 that is programmed or otherwise configured to measure AMH in samples. The computer system 201 can regulate various aspects of the methods of the present disclosure, such as, for example, the extraction and detection of cf-mRNAs in a biological sample. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220, and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives, and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, measurements of the cf-mRNAs levels as disclosed herein in a biological sample. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, determine the levels of cf-mRNAs as disclosed herein in a biological sample.

### Classifiers

The present disclosure provides classifiers for processing or analyzing data generated from a biological sample to yield an output. Such an output may result in an assessment of the cf-mRNA profile of a subject for monitoring the subject's organ or tissue before and after treatment.

A classifier may be a machine learning algorithm. The machine learning algorithm may be a trained machine learning algorithm. The machine learning algorithm may be trained via supervised or unsupervised learning, for example. For example, the machine learning algorithm may comprise generative modeling (e.g., a statistical model of a joint probability distribution on an observable variable X on a target variable Y; such as a naive Bayes classifier and linear discriminant analysis), discriminative modeling (e.g., a model of a conditional probability of a target variable Y, given an observation x of an observable variable X; such as a logistic regression, a perceptron, or a support vector machine), or reinforcement learning (RL).

As used herein, the terms “machine learning,” “machine learning procedure,” “machine learning operation,” and “machine learning algorithm” generally refer to any system or analytical and/or statistical procedure that may progressively (e.g., iteratively) improve computer performance of a task. Machine learning may include a machine learning algorithm. The machine learning algorithm may be a trained algorithm. Machine learning (ML) may comprise one or more supervised, semi-supervised, or unsupervised machine learning techniques. For example, an ML algorithm may be a trained algorithm that may be trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise, but may be not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principle component regression, least absolute shrinkage and selection operation, least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

As used herein, the terms “reinforcement learning,” “reinforcement learning procedure,” “reinforcement learning operation,” and “reinforcement learning algorithm” generally refer to any system or computational procedure that may take one or more actions to enhance or maximize some notion of a cumulative reward to its interaction with an environment. The agent performing the reinforcement learning (RL) procedure may receive positive or negative reinforcements, called an “instantaneous reward,” from taking one or more actions in the environment and therefore placing itself and the environment in various new states.

A goal of the agent may be to enhance or maximize some notion of cumulative reward. For instance, the goal of the agent may be to enhance or maximize a “discounted reward function” or an “average reward function.” A “Q-function” may represent the maximum cumulative reward obtainable from a state and an action taken at that state. A “value function” and a “generalized advantage estimator” may represent the maximum cumulative reward obtainable from a state given an optimal or best choice of actions. RL may utilize any one of more of such notions of cumulative reward. As used herein, any such function may be referred to as a “cumulative reward function.” Therefore, computing a best or optimal cumulative reward function may be equivalent to finding a best or optimal policy for the agent.

The agent and its interaction with the environment may be formulated as one or more Markov Decision Processes (MDPs), for example. The RL procedure may not assume knowledge of an exact mathematical model of the MDPs. The MDPs may be completely unknown, partially known, or completely known to the agent. The RL procedure may sit in a spectrum between the two extents of “model-based” or “model-free” with respect to prior knowledge of the MDPs. As such, the RL procedure may target large MDPs where exact methods may be infeasible or unavailable due to an unknown or stochastic nature of the MDPs.

The RL procedure may be implemented using one or more computer processors described herein. The digital processing unit may utilize an agent that trains, stores, and later on deploys a “policy” to enhance or maximize the cumulative reward. The policy may be sought (for instance, searched for) for a period of time that may be as long as possible or desired. Such an optimization problem may be solved by storing an approximation of an optimal policy, by storing an approximation of the cumulative reward function, or both. In some cases, RL procedures may store one or more tables of approximate values for such functions. In other cases, RL procedure may utilize one or more “function approximators.”

Examples of function approximators may include neural networks (such as deep neural networks) and probabilistic graphical models (e.g., Boltzmann machines, Helmholtz machines, and Hopfield networks). A function approximator may create a parameterization of an approximation of the cumulative reward function. Optimization of the function approximator with respect to its parameterization may consist of perturbing the parameters in a direction that enhances or maximizes the cumulative rewards and therefore enhances or optimizes the policy (such as in a policy gradient method), or by perturbing the function approximator to get closer to satisfy Bellman's optimality criteria (such as in a temporal difference method).

During training, the agent may take actions in the environment to obtain more information about the environment and about good or best choices of policies for survival or better utility. The actions of the agent may be randomly generated (for instance, especially in early stages of training) or may be prescribed by another machine learning paradigm (such as supervised learning, imitation learning, or any other machine learning procedure described herein). The actions of the agent may be refined by selecting actions closer to the agent's perception of what an enhanced or optimal policy is. Various training strategies may sit in a spectrum between the two extents of off-policy and on-policy methods with respect to choices between exploration and exploitation.

The trained algorithm may be configured to accept a plurality of input variables and to produce one or more output values based on the plurality of input variables. The plurality of input variables may comprise a presence or abundance of a cf-mRNA transcript corresponding to a specific gene, which the gene is organ or tissue specific. The plurality of input variables may also include clinical health data of a subject. The one or more output values may comprise a state or condition of a subject. For example, the state or condition of the subject may include one or more of: assessment of successfulness of bone marrow ablation, bone marrow reconstitution, or bone marrow transplant. Further, the state or condition of the subject may include bone marrow transplant rejection, organ donor and recipient matching, liver transplant, liver transplant rejection, lung transplant, lung transplant rejection, heart transplant, heart transplant rejection, face transplant, face transplant rejection, etc.

The trained algorithm may comprise a classifier, such that each of the one or more output values comprises one of a fixed number of possible values (e.g., a linear classifier, a logistic regression classifier, etc.) indicating a classification of a state or condition of the subject by the classifier. The trained algorithm may comprise a binary classifier, such that each of the one or more output values comprises one of two values (e.g., {0, 1}, {positive, negative}, {present, absent}, or {high-risk, low-risk}) indicating a classification of the state or condition of the subject. The trained algorithm may be another type of classifier, such that each of the one or more output values comprises one of more than two values (e.g., {0, 1, 2}, {positive, negative, indeterminate}, {present, absent, or indeterminate}, or {high-risk, intermediate-risk, low-risk}) indicating a classification of the state or condition of the subject.

The output values may comprise descriptive labels, numerical values, or a combination thereof. Some of the output values may comprise descriptive labels. Such descriptive labels may provide an identification or indication of a state or condition of the subject, and may comprise, for example, positive, negative, present, absent, high-risk, intermediate-risk, low-risk, or indeterminate. Such descriptive labels may provide an identification of a treatment for the state or condition of the subject, and may comprise, for example, a therapeutic intervention, a duration of the therapeutic intervention, and/or a dosage of the therapeutic intervention suitable to treat the state or condition of the subject. Such descriptive labels may provide an identification of secondary clinical tests that may be appropriate to perform on the subject, and may comprise, for example, a blood test, a genetic test, or a medical imaging. As another example, such descriptive labels may provide a prognosis of the state or condition of the subject. As another example, such descriptive labels may provide a relative assessment of the state or condition of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, {present, absent}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the state or condition of the subject. Some numerical values may be mapped to descriptive labels, for example, by mapping 1 to “positive” or “present,” and 0 to “negative” or “absent.”

Some of the output values may be assigned based on one or more cutoff values. For example, a binary classification of subjects may assign an output value of “positive,” “present,” or 1 if the subject has at least a 50% probability of having the state or condition. For example, a binary classification of subjects may assign an output value of “negative,” “absent,” or 0 if the subject has less than a 50% probability of having the state or condition. In this case, a single cutoff value of 50% is used to classify subjects into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of subjects may assign an output value of “positive,” “present, or 1 if the subject has a probability of having the state or condition of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of subjects may assign an output value of “positive” or 1 if the subject has a probability of having the state or condition of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of subjects may assign an output value of “negative,” absent, or 0 if the subject has a probability of having the state or condition of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of subjects may assign an output value of “negative” or 0 if the subject has a probability of the state or condition of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of subjects may assign an output value of “indeterminate” or 2 if the subject is not classified as “positive,” “negative,” “present,” “absent,” 1, or 0. In this case, a set of two cutoff values is used to classify subjects into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify subjects into one of n+1 possible output values, where n is any positive integer.

The trained algorithm may be trained with a plurality of independent training samples. Each of the independent training samples may comprise a dataset of input variables (e.g., a presence or abundance of at least one of a cf-mRNA transcripts corresponding to a gene that is organ/tissue specific collected from a subject at a given time point, and one or more known output values (e.g., a state or condition) corresponding to the subject. Independent training samples may comprise datasets of input variables and associated output values obtained or derived from a plurality of different subjects. Independent training samples may comprise datasets of input variables and associated output values obtained at a plurality of different time points from the same subject (e.g., on a regular basis such as weekly, biweekly, or monthly). Independent training samples may be associated with presence of the state or condition (e.g., training samples comprising datasets of input variables and associated output values obtained or derived from a plurality of subjects known to have the state or condition). Independent training samples may be associated with absence of the state or condition (e.g., training samples comprising datasets of input variables and associated output values obtained or derived from a plurality of subjects who are known to not have a previous diagnosis of the state or condition or who have received a negative test result for the state or condition). A plurality of different trained algorithms may be trained, such that each of the plurality of trained algorithms is trained using a different set of independent training samples (e.g., sets of independent training samples corresponding to presence or absence of different states or conditions).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise datasets of input variables associated with presence of the state or condition and/or datasets of input variables associated with absence of the state or condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the state or condition. In some embodiments, the dataset of input variables is independent of samples used to train the trained algorithm.

The trained algorithm may be trained with a first number of independent training samples associated with presence of the state or condition and a second number of independent training samples associated with absence of the state or condition. The first number of independent training samples associated with presence of the state or condition may be no more than the second number of independent training samples associated with absence of the state or condition. The first number of independent training samples associated with presence of the state or condition may be equal to the second number of independent training samples associated with absence of the state or condition. The first number of independent training samples associated with presence of the state or condition may be greater than the second number of independent training samples associated with absence of the state or condition.

A machine learning algorithm may be trained with a training set of samples from subjects with identified or diagnosed conditions, such as women with a reproductive disorder. The machine learning algorithm may be trained with at least about 5, 10, 20, 30, 40, 50, 100, 200, 300, 400, 500, 1000, or more samples. Once trained, the machine learning algorithm may be used to process data generated from one or more samples independent of samples from the training set to identify one or more features in the one or more samples (e.g., a cf-mRNA transcript level, an abundance or deficiency of a cf-mRNA transcript corresponding to a gene) at an accuracy of at least about 60%, 70%, 80%, 85%, 90%, 95%, or more. The machine learning algorithm may be used to process the data to identify the one or more features at a sensitivity of at least about 60%, 70%, 80%, 85%, 90%, 95%, or more. The machine learning algorithm may be used to process the data to identify the one or more features at a specificity of at least about 60%, 70%, 80%, 85%, 90%, 95%, or more.

The trained algorithm may be configured to identify the state or condition as disclosed herein at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the state or condition by the trained algorithm may be calculated as the percentage of independent test samples (e.g., subjects known to have the state or condition or subjects with negative clinical test results for the state or condition) that are correctly identified or classified as having or not having the state or condition.

The trained algorithm may be configured to identify the state or condition with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the state or condition using the trained algorithm may be calculated as the percentage of datasets of input variables identified or classified as having the state or condition that correspond to subjects that truly have the state or condition.

The trained algorithm may be configured to identify the state or condition with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the state or condition using the trained algorithm may be calculated as the percentage of datasets of input variables identified or classified as not having the state or condition that correspond to subjects that truly do not have the state or condition.

The trained algorithm may be configured to identify the state or condition with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the state or condition using the trained algorithm may be calculated as the percentage of independent test samples associated with presence of the state or condition (e.g., subjects known to have the state or condition) that are correctly identified or classified as having the state or condition.

The trained algorithm may be configured to identify the state or condition with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the state or condition using the trained algorithm may be calculated as the percentage of independent test samples associated with absence of the state or condition (e.g., subjects with negative clinical test results for the state or condition) that are correctly identified or classified as not having the state or condition.

The trained algorithm may be configured to identify the state or condition with an Area Under the Receiver Operating Characteristic (AUROC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUROC may be calculated as an integral of the Receiver Operating Characteristic (ROC) curve (e.g., the area under the ROC curve) associated with the trained algorithm in classifying datasets of input variables as having or not having the state or condition.

The trained algorithm may be adjusted or tuned to improve one or more of the performance, accuracy, PPV, NPV, clinical sensitivity, clinical specificity, or AUROC of identifying the state or condition. The trained algorithm may be adjusted or tuned by adjusting parameters of the trained algorithm (e.g., a set of cutoff values used to classify a dataset of input variables as described elsewhere herein, or parameters or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of features (e.g., of the input variables) may be identified as most influential or most important to be included for making high-quality classifications or identifications of the state or condition. The plurality of features or a subset thereof may be ranked based on classification metrics indicative of each feature's influence or importance toward making high-quality classifications or identifications of the state or condition. Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUROC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

### Therapeutic Targets

The detection or quantification of disease-related biological molecules (e.g., bone marrow disease-related biological markers) can be used for pre-clinical therapeutic target discovery. The detection or quantification of disease-related biological molecules can be used for pre-clinical measurement of target engagement. The detection or quantification of disease-related biological molecules can be used to track, detect, and measure targets of interest for therapy/drug discovery and development.

The detection or quantification of disease-related cell-free mRNA (e.g., bone marrow disease-related cell-free mRNA) can be used to determine gene signatures and biomarker discovery for patient stratification in pre-clinical and clinical studies.

The detection or quantification of disease-related cell-free mRNA (e.g., bone marrow disease-related cell-free mRNA) can be used to optimize late-stage lead molecule optimization for further clinical development. The detection or quantification of disease-related cell-free mRNA can be used to measure pharmacodynamics for lead optimization and clinical development during therapy/drug discovery and development. Furthermore, the detection or quantification of disease-related cell-free mRNA can be used for pharmacokinetic (PK) and safety and/or toxicity assessment. The detection or quantification of disease-related cell-free mRNA can be used to create a profile of gene expression that characterizes the pharmacodynamic effect associated with the engagement of a specific target for therapy/drug discovery and development. The detection or quantification of disease-related cell-free mRNA can be used to detect changes in pharmacodynamic target engagement for therapy/drug discovery and development.

The detection or quantification of disease related cell-free mRNA (e.g., bone marrow disease-related cell-free mRNA) can be used to measure target molecule engagement in the early clinical development of pharmaceutical candidates to treat the disease. The detection or quantification of disease related cell-free mRNA can be used in methods to select candidates for IND filings. The detection or quantification of disease related cell-free mRNA (e.g., bone marrow disease-related cell-free mRNA) can be used to measure target molecule engagement at time points periodically over a set period of time. The time points can be equal to or less than every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or any other suitable period of time. The time points can be equal or greater than every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or any other suitable period of time. The set period of time can be less than or equal to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 2 years, 3 years, 4 years, 5 years, or 10 years. The set period of time can be greater than or equal to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 2 years, 3 years, 4 years, 5 years, or 10 years.

The detection or quantification of disease related cell-free mRNA (e.g., bone marrow disease-related cell-free mRNA) can be used to develop endpoints to evaluate the relative therapeutic efficacy of therapeutic agents administered to a subject.

The development of cell-free mRNA disease signatures (e.g., cell-free mRNA bone marrow disease signatures) can be used to evaluate the relative toxicity of candidate therapeutic agents or a subject's response to therapeutic agents. For example, a subject receiving a first prescription for a first disease may then be able to be tracked closely for toxic interactions between a pharmaceutical within the first prescription administered and a candidate therapeutic by monitoring the bone marrow disease related cell-free mRNA gene panels as disclosed herein.

### EXAMPLES

### Example 1

### Different Patient Cohorts

Multiple myeloma patients eligible for autologous marrow transplantation were recruited from the Scripps Bone Marrow Transplant Center. Patients with non-secretory disease or plasma cell leukemia were excluded. Three total patients were enrolled with daily blood draws collected throughout the cytoreductive conditioning regiment and subsequent hospital stay. High-dose melphalan was used to ablate the marrow over a 2-day conditioning regiment, followed by transplantation of hematopoietic stem cells. Sequential daily collections discontinued the day of hospital discharge. Follow-up bone marrow biopsy occurred between 60-90 days. Complete blood counts (CBCs) were collected as a part of the study. Plasma was processed within 2-hours of blood collection and stored. Patient characteristics are described in Table 1.

TABLE 1
Multiple myeloma patient characteristics

| Patient | 1 | 2 | 3 |
|---|---|---|---|
| Age | 75 | 52 | 67 |
| Sex | Male | Male | Female |
| Diagnosis | IgA lambda | IgG Kappa | IgA Kappa |
| Peak relevant Ig prior to | 0.6 g/dl | 5.6 g/dl | 1.4 g/dl |
| treatment | | | gamma |
| Plasma cells at time of | 13% | 1% | <1% |
| transplant | | | |
| Prior treatment | Radiation, | Radiation, | VRD |
| | VRD | VRD | |
| Plasma cells after treatment | N/A | <0.5% | <1% |
| Relevant Ig after transplant | 0.16 g/dl | 0.8 g/dl | 0.038 g/dl |

Erythropoietin (EPO) treated patients were recruited for study enrollment provided they were administered erythropoietin as part of routine medical care. Potential patients were excluded if they were 1) currently on any anti-cancer therapy; 2) had active hemolysis from any cause, or 3) were pregnant. Patients were consented and enrolled from the Renal and Hematology/Oncology Clinics at Scripps Clinic Cancer Center. Per standard clinical care, a single dose of erythropoietin was administered per month. Blood was collected at day 0 (before administration of EPO), and at days 1, 4, and 10 after administration of EPO. Day 4 and day 10 collections were allowed for +/−1 day adjustment to accommodate patients' schedules. A subset of patients consented to an expanded protocol allowing for blood collections up to day 30. CBCs were performed as well. Cell-free hemoglobin protein (ARUP labs) and albumin levels (ARUP labs) were determined at each time point. Plasma was processed within 2-hours of blood collection and stored at −80 ° C. for batch processing. Patient characteristics are shown in Table 2.

TABLE 2
EPO patient characteristics

| Patient | 1 | 2 | 3 | 4 | 5 | 6 | 7 | 8 | 9 |
|---|---|---|---|---|---|---|---|---|---|
| Age | 84 | 67 | 82 | 91 | 73 | 78 | 80 | 74 | 80 |
| Chronic kidney disease stage | 4 | PD | 4 | 4 | 3 | 4 | 3 | 5 | 3 |
| EPO agent | Aranesp | Procrit | Aranesp | Procrit | Aranesp | Aranesp | Aranesp | Procrit | Aranesp |
| Creatinine concentration | 1.8 | 4.1 | 2.7 | 2.3 | 1.3 | 2.4 | 1.1 | 4.5 | 1.5 |
| (md/dL) | | | | | | | | | |

PD—Peritoneal Dialysis

Healthy controls. Whole blood from healthy controls was obtained from the San Diego Blood Bank. Plasma/serum was processed within 2-hours of blood collection, frozen and stored at −80 ° C. for batch processing.

G-CSF Cohort. Normal healthy individuals preparing to donate peripherally harvested stem cells for allotransplants,=were recruited from Scripps and enrolled as part of the G-CSF cohort. In total, three patients were consented and donated blood during their stem cell mobilization. Two tubes of blood were collected at day 0 (before administration of G-CSF), and at days 1, 4, and 10 after administration of G-CSF. Day 4 and day 10 collections were allowed for +/−1 day adjustment to accommodate patients' schedules and additionally, the Day 10 collection was optional. Peripheral harvest of stem cells occurred on day 4 by leukapheresis. CBCs were performed for each sample. Plasma was processed within 2-hours of blood collection and stored at −80 ° C. for batch processing. Patient characteristics are shown in Table 3.

TABLE 3
G-CSF patient characteristics

| Patient | 1 | 2 | 3 |
|---|---|---|---|
| Age | 56 | 34 | 24 |

AML Cohort. Patients with known acute myeloid leukemia (AML), in preparation for submyeloablative treatment and allogeneic stem cell transplantation as part of standard care, were recruited for daily blood draws throughout their treatment and stem cell transplant. Three patients were enrolled in the study (characteristics in Table 4), and submyeloablative treatment were generally 6-days, using a combination of fludarabine and melphalan to obtain a partial ablation of the marrow, prior to transplantation. Hematopoietic stem cells obtained from a single donor, were administered on day 0, and daily blood draws were continued through the hospital stay. In-hospital collections were limited to day 45 post-transplant. Follow-up routine bone marrow biopsies were performed. CBCs were collected as part of standard care and the data were included in the study. Plasma was processed within 2 hours of blood collection and stored for batch processing. Two of the AML patients were monitored for ˜8 weeks, while blood samples for the third patient collected until 15-day post-transplant when the patient was discharged from the hospital.

TABLE 4
AML patient characteristics

| Patient | 1 | 2 | 3 |
|---|---|---|---|
| Age | 68 | 66 | 66 |
| Sex | Female | Male | Male |
| Bone marrow blast (%) | 16 | 3 | 50 |
| Prior Therapy | Yes* | No | No |
| Additional information | | ** | |

*diffuse large B-cell lymphoma
** BM biopsy revealed lack of megakaryocyte development in Patient 2

All studies were approved by their respective institutional IRBs and patients consented according to submitted study protocols. Approval was maintained for blood collection and research through Western IRB Protocol #20162748, under which healthy control samples were collected. In collaboration with the Scripps Cancer Center and the Blood & Marrow Transplant Program at Scripps Green Hospital, G-CSF and EPO studies were conducted under Scripps Institutional Review Board approved protocol IRB-16-6808. The studies involving hematopoietic bone marrow transplants, for both multiple myeloma and acute myeloid leukemia, were approved by and conducted in accordance with Scripps IRB Protocol IRB-17-6953, in collaboration with the same groups.

### Example 2

### Sample Processing

Blood samples were collected in EDTA tubes (BD #366643) for plasma processing or in BD Vacutainer red-top clotting tubes (BD #367820) for serum processing. The biofluid used in each experiment is indicated herein as well in the corresponding cohort details in this example. Blood samples were kept at room temperature and samples processed within two hours after blood draw. Plasma and serum volume ranging from 500 μl to 1 ml was used for the extractions. Samples were first centrifuged at 1900 g for 10 min. Plasma and serum were separated into new tubes. To remove cell debris, serum/plasma was subsequently centrifuged at 16000 g. For cancer patient plasma samples (multiple myeloma and AML) the second centrifugation step was performed at 6000 g. Plasma/serum samples were immediately frozen and stored at −80 ° C. Freeze/thaw cycles were avoided. Buffy coat samples were obtained by isolating the buffy coat layer enriched in white blood cells after initial centrifugation of blood samples. Nucleic acids were isolated from plasma/serum using the Circulating Nucleic Acid kit (Qiagen). ERCC RNA Spike-In Mix (Thermo Fisher Scientific, Cat. #4456740) was added during the extraction process as an exogenous spike-in control according to manufacturer's instruction (Ambion). Nucleic acids from whole blood and buffy coat samples were extracted with TRIzol LS (ThermoFisher) following the manufacturer instructions. Subsequently, RNA and cf-RNA samples were incubated for 25 minutes with 3 μl of the inhibitor resistant rDNase (Turbo DNase, Invitrogen) to eliminate any remnant DNA and concentrated afterwards. RNA was eluted in 15 μl of RNase free water. The amount, size, and integrity of cfRNA was estimated by running 1 μl of the sample in an Agilent RNA 6000 Pico chip using a 2100 Bioanalyzer (Agilent Technologies) and confirmed by B-actin qPCR. 25-30% of the cf-RNA eluate was converted to cDNA, using random hexamers and NGS libraries were generated and exome capture performed for Illumina sequencing. Libraries were quantified by qPCR with Kapa quantification kit (Kapa) and in a Quantifluor (Agilent Quantus Fluorometer, Promega) using QuantiFluor ONE dsDNA kit (Promega), and library size was checked on the Bioanalyzer (Agilent Technologies) using high sensitivity DNA chips (Agilent Technologies). Samples were pooled and sequenced on a NextSeq 500 (Illumina) platform according to manufacturer's instructions.

### Example 3

### Sequence Data Processing, Alignment, and Transcriptome Quantification

Base-calling was performed on an Illumina BaseSpace platform, using the FASTQ Generation Application. Adaptor sequences are removed, and low quality bases trimmed, using cutadapt (v1.11). Reads shorter than 15 base-pairs were excluded from subsequent analysis. Read sequences are then aligned to the human reference genome GRCh38 using STAR (v2.5.2b) with GENCODE version 24 gene models. Duplicated reads are removed by invoking the samtools (v1.3.1) rmdup command. Gene expression levels were inferred from de-duplicated BAM files using RSEM (v1.3.0).

### Example 4

### Differential Expression Analysis

Differential expression analysis between different conditions was performed using DESeq2 (v1.12.4). RSEM-estimated read counts are used as input for DESeq2. Genes with fewer than 20 reads across the samples are excluded from this analysis. Potential Gene Ontology enrichment of differentially expressed genes were examined using the R package limma (v3.28.21).

### Example 5

### Tissue/Cell-Type Specific Genes

Tissue (cell-type) specific genes are defined as genes that show much higher expression in a particular tissue (cell-type) compared to other tissues (cell-types). Information about tissue (cell-type) transcriptome expression levels was obtained from the following two public databases: GTEx (www.gtexportal.org/home/) for gene expression across 51 human tissues and Blueprint Epigenome (www.blueprint-epigenome.eu/) for gene expression across 56 human hematopoietic cell types. For each gene, the tissues (cell-types) were ranked by their expression of that particular gene and if the expression in the top tissue (cell-type) is >20 fold higher than all the other tissues (cell-types) the gene was considered specific to the top tissue (cell-type). For the establishment of BM enriched transcripts, human BM RNA was purchased from ThermoFisher and performed RNA-seq. Subsequently, BM transcriptome was compared to whole blood transcriptome to identify genes enriched in BM and WB transcriptomes (fold change >5).

### Example 6

### Immunoglobulin Gene Repertoire in Multiple Myeloma Patients

For clone-type assembly, de novo transcriptome assembly was performed using Trinity. Next, the assembled contigs were compared to immunoglobulin gene annotation database IMGT (www.imgt.org/) using igBLAST (v2.5.1) to identify the V(D)J combinations. To quantify the relative abundance of variable region genes, reads that were either unaligned to the human reference genome or aligned to an annotated Ig gene by STAR were collected and mapped sequences in the IMGT database using igBLAST. Relative abundance was calculated as the ratio of number of reads mapped to a particular Ig gene over the total number of reads mapped to any Ig gene.

### Example 7

### Unsupervised Clustering of Multiple Myeloma and AML Samples

Genes that met the following two criteria were selected for clustering: 1) the maximum expression across time points higher than 50 TPM (transcripts per million) and 2) the ratio of the highest expression over the lowest was greater than 5. For each of the selected genes, the expression values were normalized by dividing each value by the maximum value across all time points. The purpose of this normalization was to bring all the genes to a comparable scale and focus on their relative changes across time points instead of their absolute expression levels. K-means and hierarchical clustering were then performed to find genes that share similar temporal expression patterns.

### Example 8

### Decomposing Data with Non-Negative Matrix Factorization (NMF)

Genes whose expression was lower than 20 TPM in all samples were excluded from the decomposition analysis. For each of the remaining genes, the expression values were normalized by dividing each value by the maximum value across all samples. The purpose of this normalization step is to bring all the genes to a comparable scale. NMF was then performed on the normalized values to decompose the genes into 8-12 components. NMF decomposition was implemented by invoking the “decomposition.NMF” class in the sciki-learn Python library. NMF decomposition creates groups of genes (components) sharing similar expression patterns (correlated across samples) in an un-supervised manner, thereby revealing underlying structures within the data. In order to better annotate the discovered components, genes enriched in a particular component (i.e., those genes that have the highest loadings within the component) were selected and examined for: 1) their expression levels across 51 human tissues in GTEx; 2) their expression levels across 55 human hematopoietic cell types from the Blueprint Epigenome consortium; and 3) their Gene Ontology functional enrichment. If most of these genes showed high expression in a certain cell type (e.g., platelet) or were enriched in certain biological processes (e.g., “platelet activation” and “coagulation”), the component were designated accordingly (e.g., calling it “megakaryocyte component”). By integrating those three sources of information, the tissue/cell-type origin for most components were able to be ascertained.

### Example 9

### cf-mRNA Transcriptome is Enriched in Hematopoietic Progenitor Transcripts

To characterize the landscape of the human cell-free RNA transcriptome, cf-mRNA from 1 ml of serum of 24 healthy donors was isolated and sequenced. Among this cohort, 10,357 transcripts with >1 TPM (transcripts per million) and 7,386 transcripts with >5 TPM in at least 80% of the samples were identified, reflecting the diversity and consistency of cf-mRNA transcriptome among healthy subjects.

TABLE 5
Average number of transcripts detected
in cf-mRNA of healthy donors (n = 24)

| TPM | >40% of | >60% of | >80% of |
|---|---|---|---|
| Criteria | the samples | the samples | the samples |
| TPM >1 | 12341 | 11393 | 10313 |
| TPM >5 | 9414 | 8485 | 7334 |

TABLE 6
Summary of sequencing metrics

| | Reads aligned | Reads aligned | Duplication | Correlation with | Unique | Protein coding |
|---|---|---|---|---|---|---|
| Sample ID | to mRNA (%) | to intron (%) | rate | ERCC (PCC) | fragment | genes detected* |
| 12687-A1 | 64.9 | 7.7 | 10.5 | 0.95 | 306643 | 10183 |
| 12687-A2 | 70.6 | 5.6 | 7.4 | 0.97 | 264871 | 9718 |
| 12819-A1 | 87.2 | 1.2 | 13.3 | 0.94 | 314330 | 9652 |
| 12819-A2 | 89.5 | 1.8 | 13.5 | 0.93 | 397425 | 10204 |
| 12824-A1 | 82.8 | 3.5 | 14.3 | 0.96 | 552282 | 11007 |
| 12824-A2 | 91.9 | 1.2 | 9.2 | 0.93 | 583604 | 11106 |
| 12829-A1 | 90.0 | 1.5 | 12 | 0.96 | 473651 | 10561 |
| 12829-A2 | 90.5 | 1.2 | 11.4 | 0.89 | 492788 | 10691 |
| 12835-A1 | 94.5 | 1.1 | 11.9 | 0.96 | 861572 | 12118 |
| 12835-A2 | 89.0 | 1.9 | 10.1 | 0.95 | 757347 | 12028 |
| 12841-A1 | 87.2 | 2.6 | 17.6 | 0.91 | 524589 | 10742 |
| 12841-A2 | 94.3 | 1.1 | 10.2 | 0.98 | 774486 | 11587 |
| 12846-A1 | 90.1 | 1.2 | 16.2 | 0.92 | 591508 | 11196 |
| 12846-A2 | 93.7 | 1.2 | 12.2 | 0.93 | 604647 | 11248 |
| 12852-A1 | 90.5 | 1.9 | 11.7 | 0.89 | 433837 | 10251 |
| 12852-A2 | 90.7 | 1.8 | 7.4 | 0.88 | 412466 | 10168 |
| 12858-A1 | 89.9 | 2.3 | 24 | 0.93 | 839497 | 11886 |
| 12858-A2 | 91.3 | 1.8 | 20.9 | 0.92 | 676180 | 11351 |
| 12864-A1 | 88.7 | 2.3 | 8 | 0.97 | 474861 | 10933 |
| 12864-A2 | 88.9 | 2.3 | 5.1 | 0.97 | 442572 | 10784 |
| 13079-A1 | 84.5 | 3.3 | 4.6 | 0.97 | 474443 | 10455 |
| 13079-A2 | 84.8 | 3.3 | 3.2 | 0.91 | 422299 | 10224 |
| 13086-A1 | 89.9 | 2.1 | 5.9 | 0.97 | 657814 | 11390 |
| 13086-A2 | 90.1 | 2.1 | 3.8 | 0.96 | 593309 | 11221 |
| 13092-A1 | 85.9 | 1.2 | 14 | 0.96 | 605880 | 11036 |
| 13092-A2 | 89.2 | 1.5 | 8.7 | 0.91 | 376971 | 10101 |
| 13096-A1 | 88.5 | 2.0 | 13.6 | 0.93 | 311271 | 9952 |
| 13096-A2 | 88.6 | 2.0 | 8.5 | 0.93 | 298347 | 9799 |
| 13103-A1 | 76.2 | 5.0 | 13.5 | 0.96 | 471299 | 10361 |
| 13103-A2 | 80.0 | 3.7 | 13.5 | 0.95 | 366955 | 9803 |
| 13110-A1 | 78.3 | 4.7 | 4.2 | 0.95 | 1520926 | 12952 |
| 13110-A2 | 91.2 | 2.1 | 3.2 | 0.88 | 1792888 | 13193 |
| 13120-A1 | 78.6 | 4.3 | 8.9 | 0.96 | 399780 | 9493 |
| 13120-A2 | 81.4 | 1.3 | 12.6 | 0.95 | 492775 | 9751 |
| 13126-A1 | 92.0 | 1.1 | 20.9 | 0.96 | 444705 | 10655 |
| 13126-A2 | 91.4 | 1.0 | 19.9 | 0.92 | 435998 | 10760 |
| 13129-A1 | 71.3 | 6.4 | 6 | 0.96 | 478551 | 10784 |
| 13129-A2 | 88.3 | 2.4 | 5 | 0.95 | 656115 | 11371 |
| 13136-A1 | 85.2 | 1.4 | 8.2 | 0.95 | 510213 | 10924 |
| 13136-A2 | 85.0 | 2.6 | 6 | 0.94 | 581233 | 11260 |
| 4510-A1 | 73.4 | 2.8 | 6.6 | 0.92 | 738901 | 12253 |
| 4510-A2 | 67.2 | 1.2 | 12 | 0.96 | 328331 | 10189 |
| 9709-A1 | 91.0 | 1.0 | 8.6 | 0.93 | 991082 | 12406 |
| 9709-A2 | 81.0 | 3.3 | 8.7 | 0.95 | 827893 | 12377 |
| 9737-A1 | 90.8 | 0.7 | 6.3 | 0.96 | 1331072 | 12857 |
| 9760-A1 | 87.4 | 1.0 | 15.1 | 0.91 | 828881 | 12256 |
| 9760-A2 | 78.1 | 3.0 | 14.4 | 0.96 | 468786 | 11064 |

*TPM is greater than equal to 2. A1 and A2 denote replicates.
PCC: Pearson's correlation coefficient

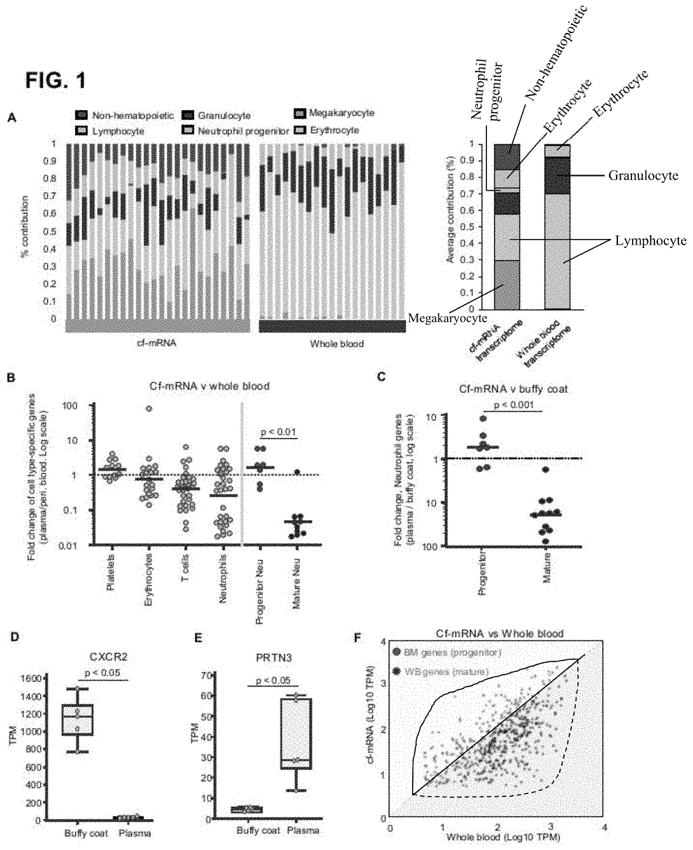

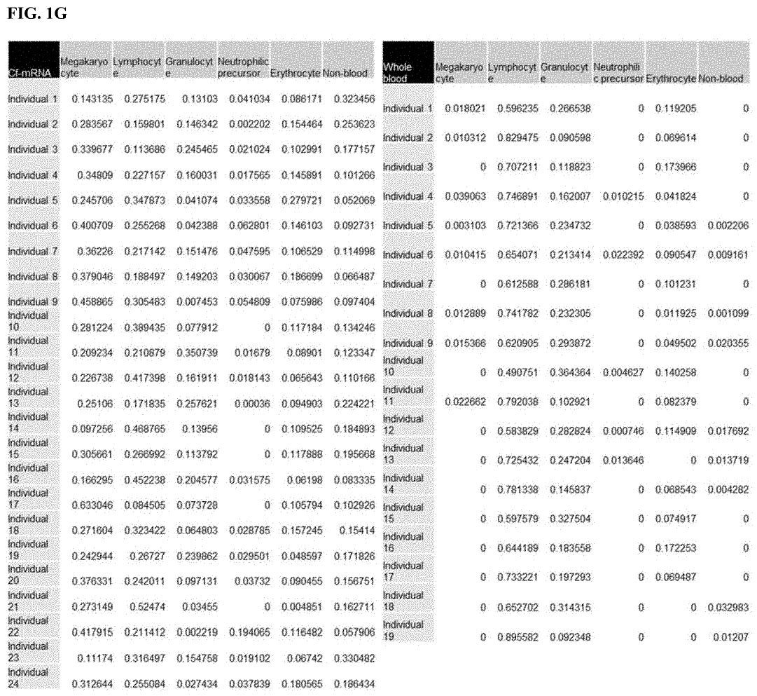

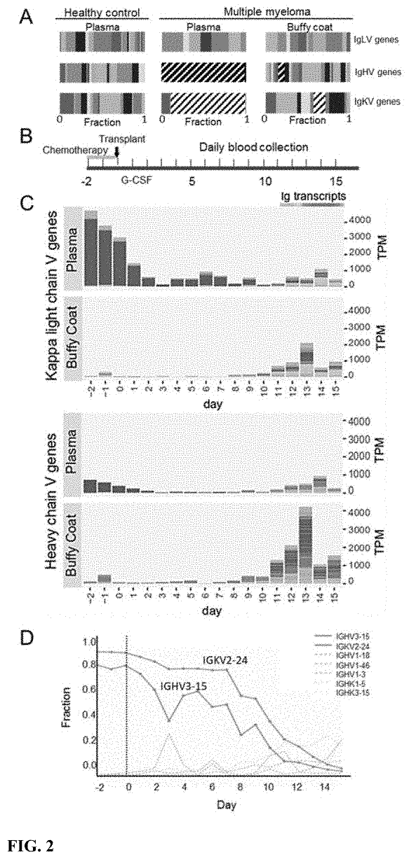

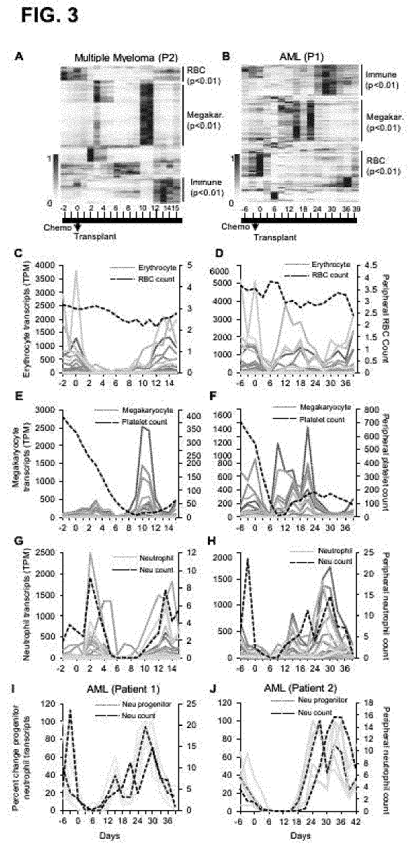

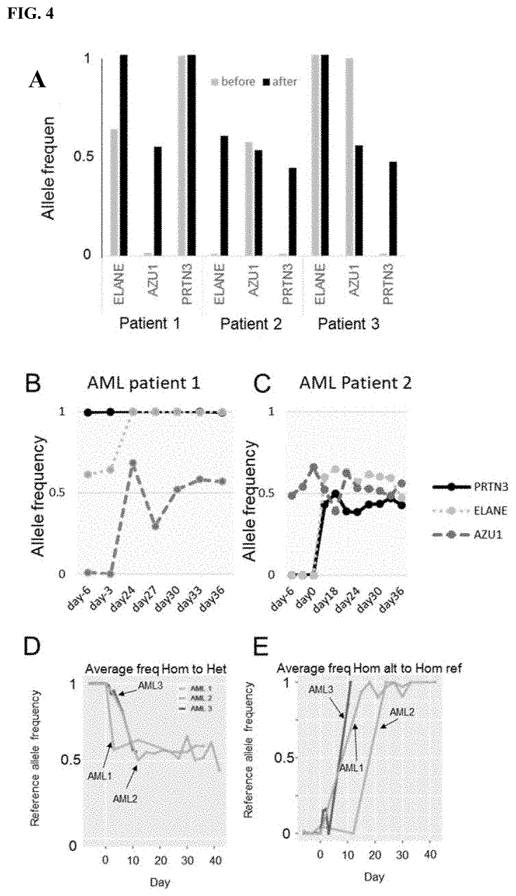

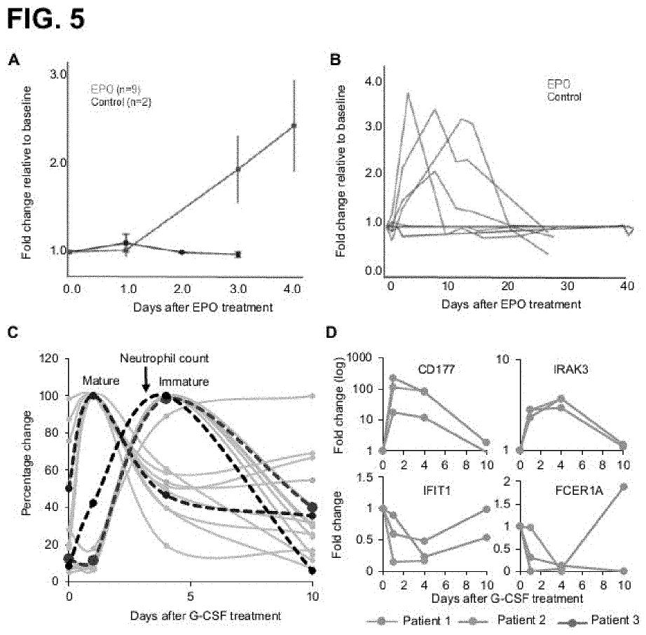

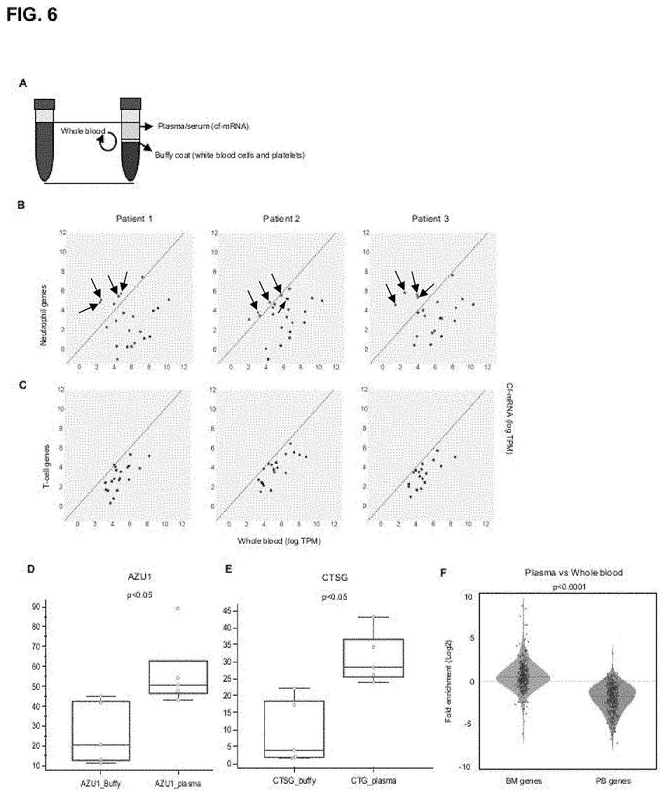

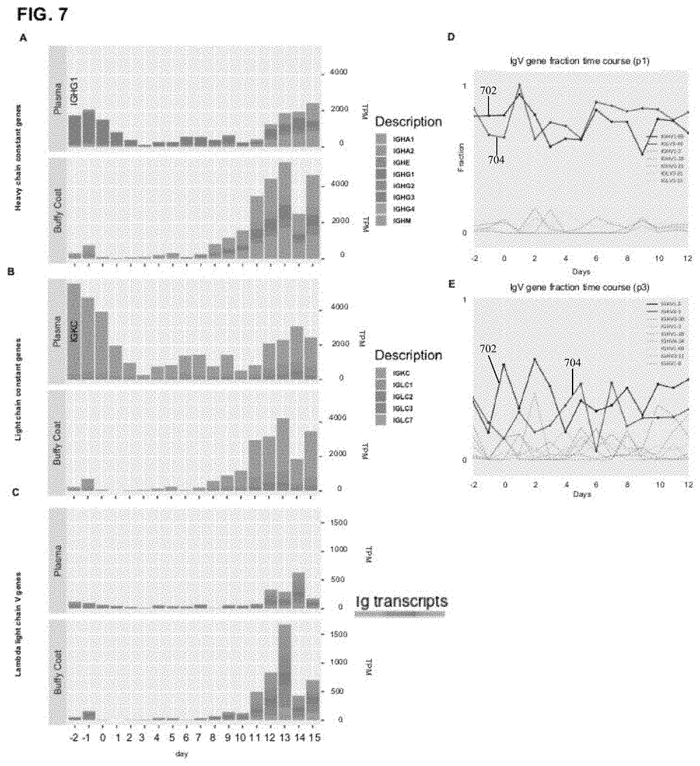

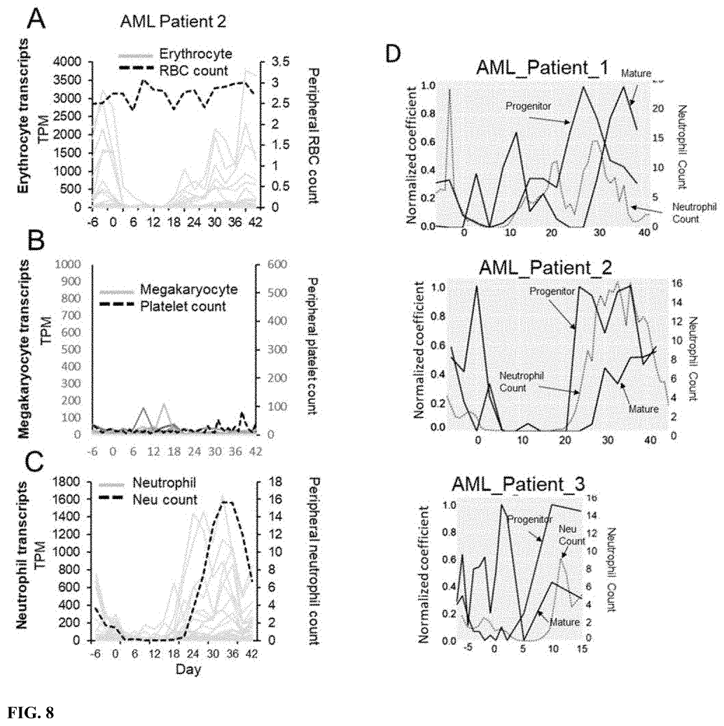

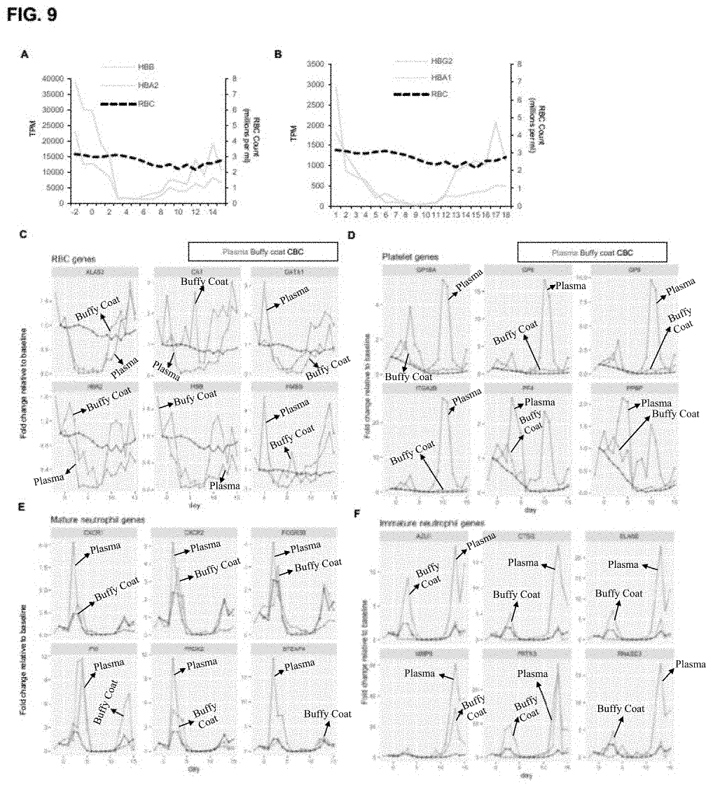

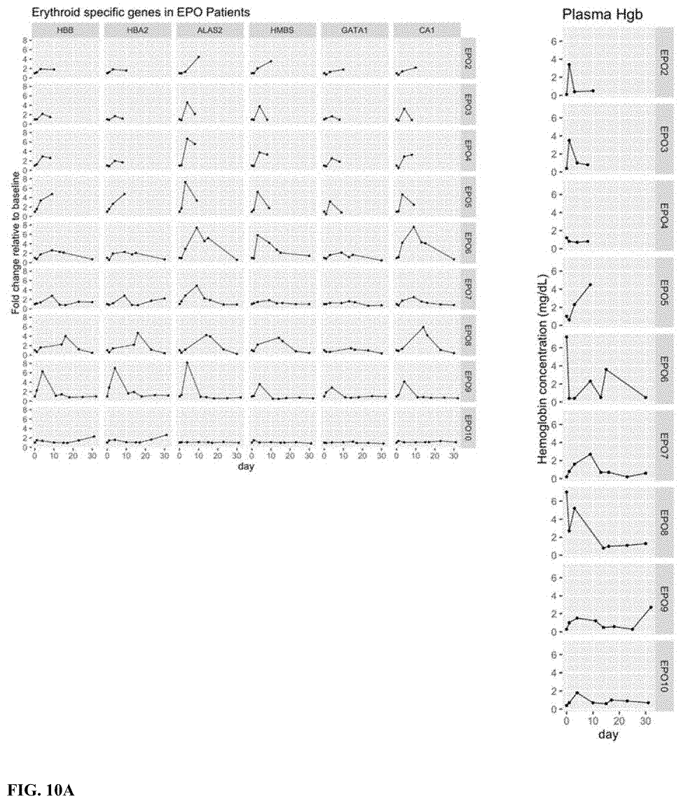

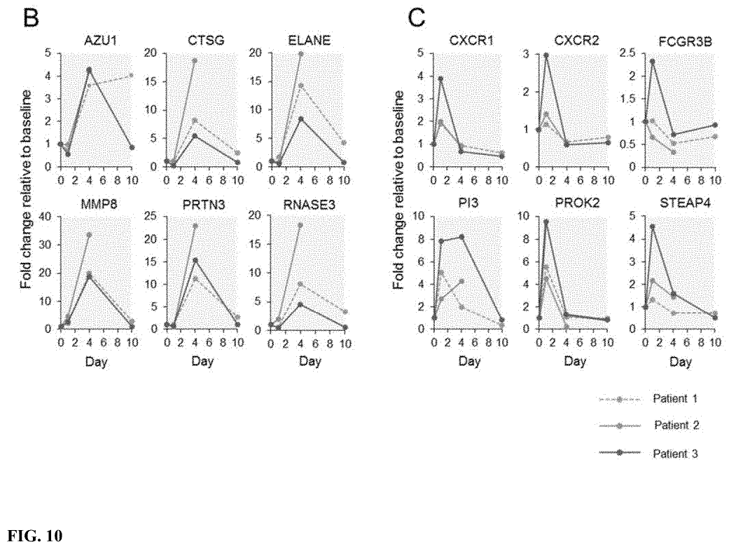

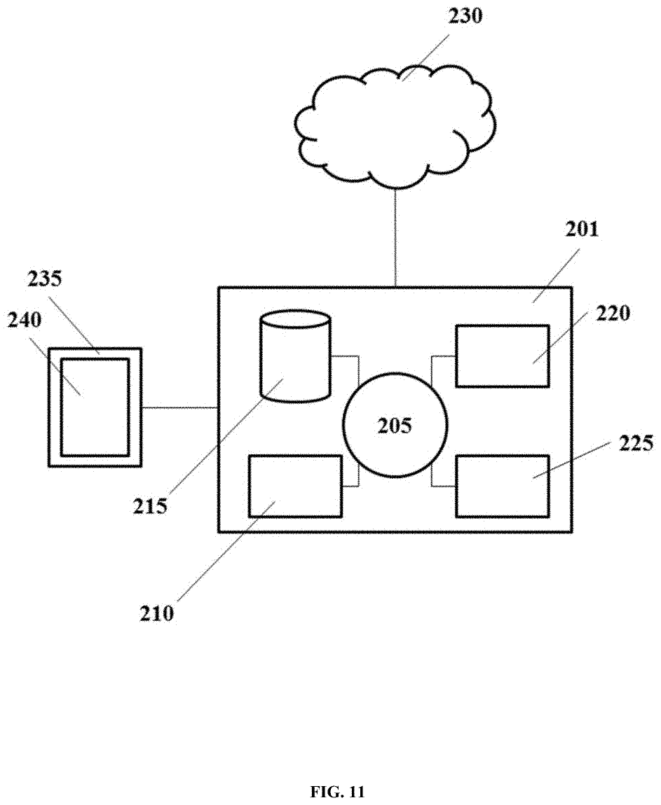

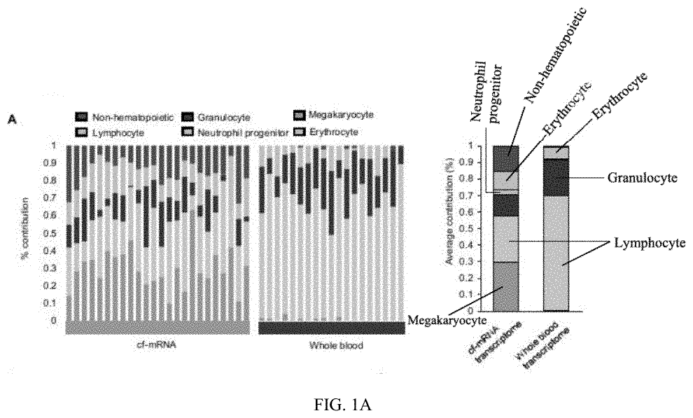

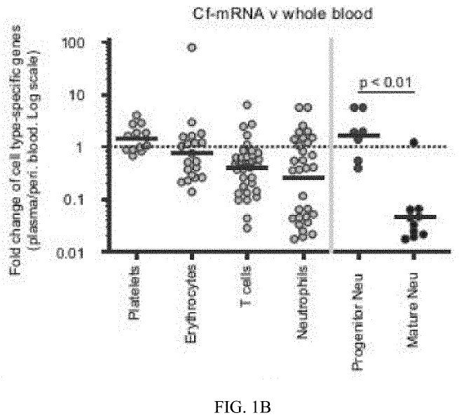

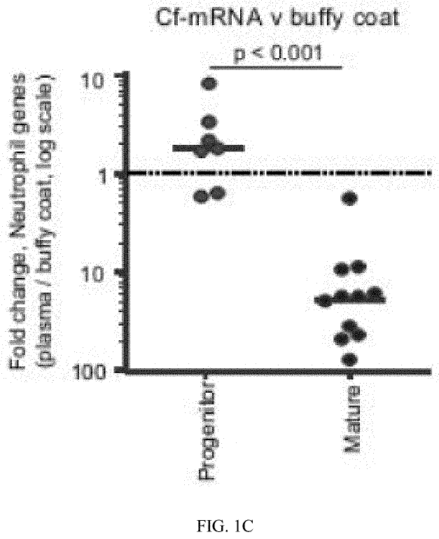

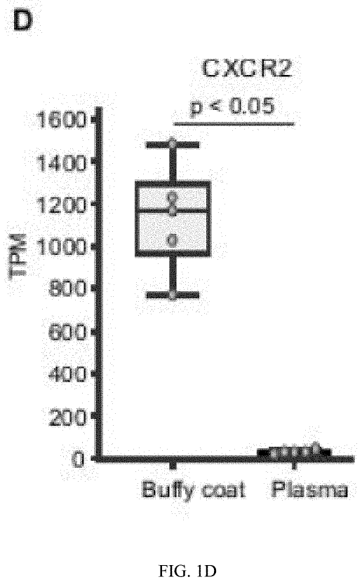

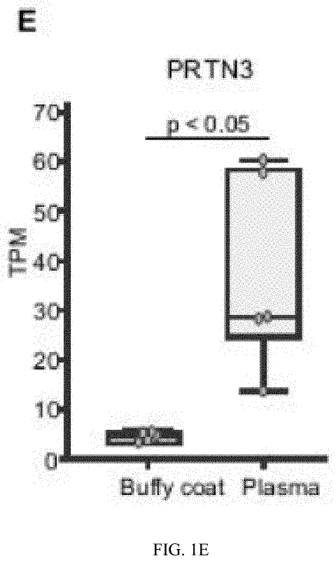

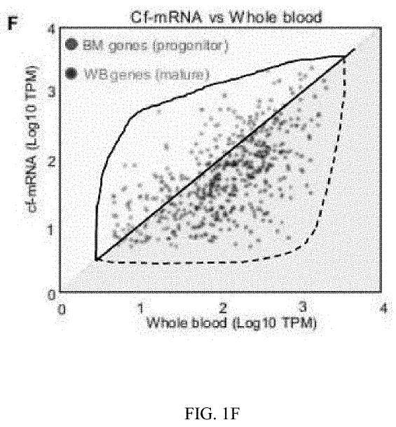

Non-negative matrix factorization was used to decompose the cf-mRNA transcriptome in an unsupervised manner and gene expression reference databases (GTEx and Blueprint) to estimate the relative contributions of the different tissues and cell types (see Material and Methods). The majority of the transcripts detected in cf-mRNA, ˜85% on average, are of hematopoietic origin (i.e., derived from circulating cells and BM-residing cells), with the remaining ˜15% being of non-hematopoietic origin (i.e., derived from solid tissues, FIG. 1A). Specifically, deconvolution analyses estimated that, on average, ˜29% of transcripts are of megakaryocyte/platelet origin (first to third quartile range 23-36%), ˜28% are of lymphocyte origin (range 18-30%), 12.8% of granulocyte origin (range 6-16%), 3% of neutrophil progenitor origin (range 0.2-3.7%), 11% of erythrocyte origin (range 8-14%) and ˜15% derived from solid tissues (range 11-20%). (FIG. 1A). To gain insights into the origin of these transcripts, similar deconvolution analysis was performed in whole blood samples from 19 healthy individuals from previously reported RNA-Seq data. As expected, the whole blood transcriptome is largely composed of lymphocyte (˜69% on average) and granulocyte (˜22% on average) transcripts, with an additional ˜7% of transcripts of erythrocyte origin and minor contributions from other cell types and tissues (FIG. 1A). These analyses represent an estimation of the composition of the transcriptome of these biofluids that could be influenced by different factors. Nevertheless, the data shows the higher diversity of cf-mRNA transcriptome, which, compared to whole blood, contains a larger fraction of non-hematopoietic transcripts and of hematopoietic progenitor genes derived from the BM.

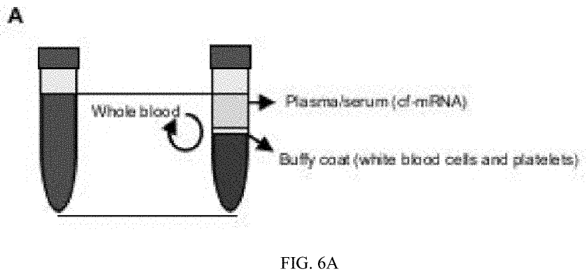

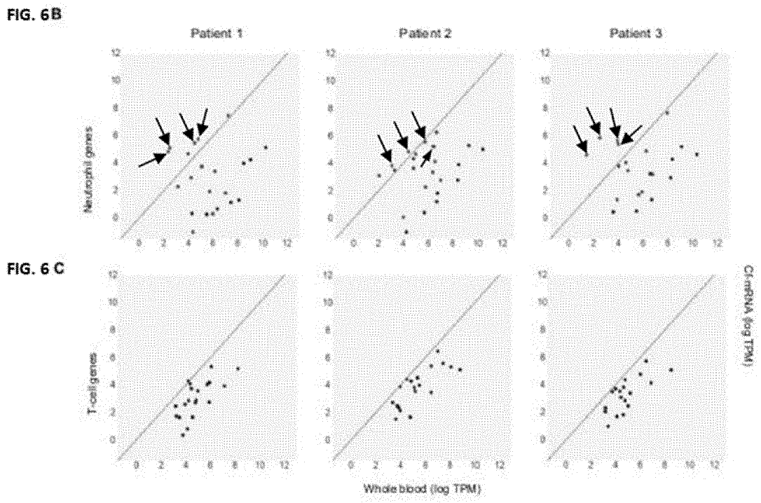

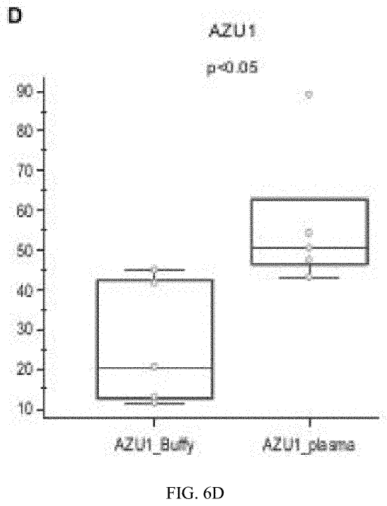

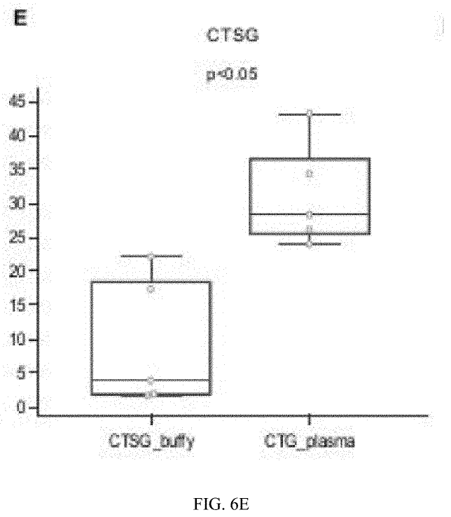

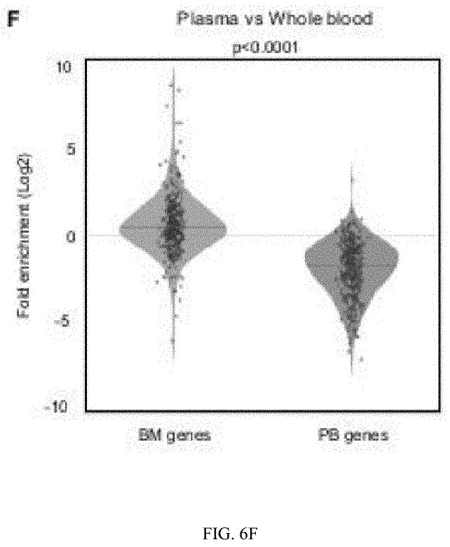

To confirm the presence of BM-specific transcripts in circulation, RNA-Seq was performed in 3 paired whole blood (which includes all cellular components of blood) and plasma samples from healthy donors (FIG. 6A) and compared the levels of the main hematopoietic cell type-specific transcripts (i.e., neutrophils, erythrocytes, platelets/megakaryocyte, T cells) in these specimens (FIG. 1B, FIG. 6B-C). Striking differences were observed among neutrophil-specific transcripts (FIG. 1B). Using the hematopoiesis transcriptomic reference database (Blueprint), transcripts expressed in mature circulating neutrophils were detected at much lower levels in plasma compared to whole blood (FIG. 1B). In contrast, transcripts expressed in BM-residing neutrophil progenitors were highly enriched in cf-mRNA (FIG. 1B). To confirm these findings, RNA-Seq of five paired plasma and buffy coat samples (buffy coat is enriched in white blood cells) was performed. Consistently, neutrophil mature and progenitor transcripts were found to form distinct populations (FIG. 1C), in which cf-mRNA shows low levels of mature transcripts such as the chemokine receptors CXCR1 and CXCR2 (FIG. 1D, p<0.01) compared to buffy coat, but enriched in progenitor transcripts such as PRTN3 (myeloblastin precursor), CTSG (cathepsin G) and AZU1 (azurocidin precursor) (p<0.05, FIG. 1E, FIGS. 6D and 6E). These data support the presence of BM transcripts in cf-mRNA; indeed, quadratic programming deconvolution analysis of hematopoietic transcripts from healthy donors indicated that BM transcripts contribute ˜9% of cf-mRNA transcriptome, in contrast to ˜1% in whole blood.

To further confirm this result, RNA-seq on a human BM sample was performed and compared it with the whole blood transcriptome. 377 genes enriched in BM transcriptome (>5 fold, “BM genes”) were identified as listed in Table 7 below, representing hematopoietic progenitors (i.e., neutrophil progenitors and mesenchymal stem cells from the BM). Progenitor transcripts such as PRTN3, CTSG, and AZU1 are among the top transcripts enriched in BM transcriptome. In addition, 374 genes were identified enriched in whole blood (>5 fold, “WB genes”) (Table 8), representing mature circulating blood cell genes, as expected (i.e., associated with mature granulocytes and lymphocytes). Subsequently, the levels of “BM genes” and “WB genes” were compared in three matching whole blood and plasma samples, which confirmed that these transcripts segregate into two populations (p<0.001), with cf-mRNA being enriched in hematopoietic progenitor genes (“BM genes”) and “depleted” of mature genes (“WB genes”) compared to whole blood (FIG. 1F and FIG. 6F). In summary, the data indicate that cf-mRNA transcriptome captured transcripts derived from the BM, providing a window to non-invasively evaluate BM function.

TABLE 7
List of bone marrow enriched genes compared to whole blood
Gene ID

| | | | | | |
|---|---|---|---|---|---|
| PRTN3 | HIST1H2BM | IGFBP5 | HIST1H1C | COL1A2 | PIF1 |
| CTSG | HES6 | CRYAB | CEACAM1 | CDC6 | INCENP |
| ELANE | APOD | ACTC1 | SAA2 | ATP2C2 | TCF19 |
| MPO | MYH7 | SERPINB10 | CTD-2116N17.1 | NEK2 | C1orf228 |
| DEFA4 | LPL | METTL12 | CDC25A | RGL4 | PADI4 |
| MMP8 | CCNA2 | FGFR1 | MMP2 | BGN | TIMELESS |
| CD177 | UBE2C | GPR84 | DEPDC1B | FOLR3 | GAS6 |
| CXCL12 | RP11-84C10.2 | CEBPE | GPX3 | SLC1A3 | STOM |
| OLFM4 | SLPI | PTX3 | HIST1H2BG | RRM1 | UBE2S |
| AZU1 | CCNB2 | SRGN | NOCT | APOA1 | SLC43A1 |
| DEFA3 | TF | SHCBP1 | ERLIN1 | SMC2 | TICRR |
| LTF | PKMYT1 | DTL | LRP3 | TUBG1 | COX6A2 |
| CEACAM8 | KIF2C | PLPPR3 | PLEKHH3 | DLC1 | MCM10 |
| HIST1H3B | RP11-872D17.8 | SPTA1 | MAD2L1 | HIST1H2BJ | IL1R1 |
| RNASE3 | TOP2A | HMGB2 | ASNS | HELLS | SPARCL1 |
| MS4A3 | MCM2 | KIF20A | ORC1 | HK3 | IGFBP4 |
| HIST1H1B | PKLR | IGFBP7 | NCAPG | VAT1 | CENPP |
| CEACAM6 | ERG | GTSE1 | MS4A4A | FMO2 | ALDH4A1 |
| FAM132B | TK1 | SAA1 | SLC15A2 | TFRC | CAPN3 |
| PRG2 | CLEC5A | HIST1H2BL | ENTPD7 | FBN1 | DHCR24 |
| RETN | SPP1 | KPNA2 | IQGAP3 | ADCY6 | MSH5 |
| CLEC11A | HIST1H2AH | CENPM | WDR34 | NLRC4 | HNRNPAB |
| BPI | OLR1 | PLK4 | MGST1 | IGHV4-59 | SLC28A3 |
| RMRP | AURKB | HIST1H2AM | HIST2H2AB | DZIP1L | C1S |
| CHIT1 | FBLN1 | CDCA7L | FAM178B | FANCI | MLNR |
| RRM2 | IGHV4-39 | CKAP2L | FUT4 | GSG2 | GADD45A |
| HIST1H3J | HIST1H4J | HIST1H2AD | LBP | HIST1H2BE | |
| PRSS57 | CDCA3 | AQP1 | ITGA9 | HMMR | |
| LCN2 | SPAG5 | CDC45 | GRB10 | IARS | |
| TCN1 | ALB | IGLC2 | ANKRD18A | MTFR2 | |
| ABCA13 | HIST1H2BB | TPX2 | DCN | HIST1H2AE | |
| RNASE2 | CDCA5 | ARHGAP11A | FBXO5 | ARHGAP33 | |
| ANXA3 | KIFC1 | NXF3 | BUB1B | CLSPN | |
| HIST1H3C | UHRF1 | ARG1 | ANKLE1 | FEN1 | |
| TYMS | FOXM1 | CP | GALNT14 | FAM83D | |
| PRRT4 | KIF18B | SYNGR1 | DNAH10 | CHEK1 | |
| HIST1H3F | ESPL1 | GGH | HTRA3 | FAM201A | |
| EPX | S100A12 | PCOLCE2 | FGA | GABBR1 | |
| CD24 | SPC24 | BCL2L15 | KIF23 | CDCA2 | |
| APOE | RP4-781K5.2 | DES | MTL5 | DHCR7 | |
| MKI67 | IGFBP2 | PSAT1 | RNU11 | KNTC1 | |
| HP | IGLL1 | CPNE3 | CHL1 | SERPINH1 | |
| HIST1H2AB | H2AFX | C1QA | NUF2 | CTSL | |
| HIST1H3G | S100A8 | MROH6 | HMBS | STEAP3 | |
| ACTA1 | MYL2 | PHGDH | HIST1H2BO | GMNN | |
| SLC2A5 | HSPA1B | FSTL3 | PIWIL4 | MYBL2 | |
| CDT1 | HIST1H4I | CENPA | NT5DC4 | PIGQ | |
| ATP8B4 | S100A9 | HBA1 | SERPINB1 | DEPDC1 | |
| KIAA0101 | MB | TARM1 | KIAA1524 | COL6A1 | |
| NNMT | PLTP | FAM46A | HIST1H4C | RAD51 | |
| HIST1H2AL | UGCG | TCTEX1D1 | COL1A1 | TTK | |
| CDC20 | HIST1H2AI | CITED4 | RP11-867G2.8 | SGOL2 | |
| BIRC5 | VCAM1 | PTTG1 | SLC1A4 | MATR3 | |
| HBD | MLC1 | KIF4A | SLC22A31 | CENPO | |
| C7 | HIST1H3I | CENPF | FGG | HSPB7 | |
| HIST1H4D | PCNA | UBE2T | RHAG | MCM6 | |
| C1QC | CCL14 | CENPE | FN1 | MTHFD1L | |
| BEX1 | MCM4 | SMC4 | KIF11 | PYCR1 | |
| HJURP | FGB | CCNF | ITGA7 | POLE | |
| HIST1H2AJ | GFI1 | HIST1H1D | H1F0 | FAR2 | |
| NUCB2 | CDK1 | ASPM | VEGFA | CST7 | |
| MMP9 | STMN1 | NDC80 | RHCE | DOC2B | |
| CAMP | PRG3 | MICALL2 | IGHV4-4 | HMGB3 | |
| PLK1 | PRC1 | RECQL4 | LDHA | ZWINT | |
| PGLYRP1 | MYB | CDCA7 | PKP2 | TUBB | |
| CRISP3 | HIST1H2AG | CKS2 | CENPU | RP11-65L3.2 | |
| SEPP1 | TNC | NCAPG2 | IGFBP3 | RAD54L | |
| C1QB | CA1 | TACSTD2 | E2F8 | TTN-AS1 | |
| KCNH2 | CLTCL1 | ADD2 | RNASE1 | WEE1 | |
| CIT | HIST1H4A | MCEMP1 | ARHGAP23 | EPAS1 | |

TABLE 8
List of genes enriched in whole blood compared to bone marrow
Gene ID

| | | | | |
|---|---|---|---|---|
| CXCL8 | FAM46C | IL10RA | ASCC2 | GZMA |
| TREM1 | GIMAP4 | CTC-250I14.6 | CXCR1 | RASA3 |
| PHOSPHO1 | ZDHHC18 | CASP1 | TMIGD2 | ABTB1 |
| TCF7 | SPON2 | RP11-195O1.5 | ATP6V0E2 | FBXW7 |
| SECTM1 | SGK1 | CDC42EP2 | EPHA4 | PLEKHB1 |
| MME | CRIP1 | FGL2 | UBE2B | ALPL |
| ALS2CR12 | TNFRSF10C | AC090498.1 | PTAFR | LRRC25 |
| PTGS2 | SPOCK2 | CXCR3 | BHLHE40 | TNFAIP2 |
| PRF1 | TRANK1 | ADIPOR1 | CHST15 | 1-Sep |
| PCGF5 | TUBB1 | WDR60 | SRPK2 | PPP2R5B |
| DNAJC6 | AMICA1 | CMTM2 | GIMAP5 | RNASE6 |
| SULF2 | BPGM | FOS | IFIT2 | RP11-53I6.2 |
| CTA-363E6.6 | ARHGEF3 | PTPRC | PTPRE | PCED1B |
| PDZK1IP1 | GZMB | AMPD2 | FCER1A | ARRB1 |
| RP4-576H24.4 | HLA-DRB1 | UBXN6 | CACNA2D4 | RAPGEF1 |
| IL2RB | DCAF12 | LIMD2 | CCR3 | PTCH1 |
| BAG1 | CD6 | PIK3IP1 | FAM65B | NHSL2 |
| RAB2B | VWCE | PRR5L | PPM1F | ABCG1 |
| CXCR2 | FCGR3B | EPB41 | CST3 | SLC11A1 |
| HLA-DPB1 | IL32 | IGF2BP3 | TNFSF12 | LITAF |
| TMEM56 | AQP9 | IRF1 | RARA-AS1 | CD2 |
| CD5 | FLT3LG | TSPAN5 | KRT23 | GPBAR1 |
| GIMAP1-GIMAP5 | KIAA1324 | LDLRAP1 | MMD | SLC15A3 |
| GNLY | YPEL3 | AHNAK | CTSW | MICAL2 |
| RGS2 | PTPRA | LYPD2 | DNAJB2 | SWT1 |
| CCL4L2 | ITGB7 | OAZ2 | ENKUR | NAAA |
| ADGRE2 | CCL4 | ZFP36L1 | APOL3 | SKI |
| FGFBP2 | KLRB1 | KLRK1 | FAM102A | BEST1 |
| RARA | PPBP | ADGRE5 | CSF1R | PROK2 |
| CYTIP | RUNDC3A | DGAT2 | SGSH | ALOX15 |
| CX3CR1 | CAMK4 | SH2D2A | PSMB9 | ZHX2 |
| DOK2 | PTPRCAP | CASS4 | VMP1 | RP11-598P20.5 |
| SHISA4 | ZNF385A | R3HDM4 | NLRP1 | RP11-22N19.2 |
| CD7 | SIGLEC10 | RBM23 | CDYL | CTSS |
| PI3 | IL7R | HLA-DMB | RUNX3 | GLIPR1 |
| CD52 | IL12RB1 | CD300A | C9orf78 | PPM1A |
| CLEC7A | NINJ1 | RANBP10 | TMCC1 | YY1AP1 |
| CCL5 | AC004076.9 | AGO2 | PACS1 | PREX1 |
| GATA3 | ADGRE3 | EIF1AY | PPCDC | PDZD4 |
| BNIP3L | SAMD3 | OR2W3 | AKNA | RAB8A |
| FCHO2 | BCL2L1 | NCR3 | SPARC | STRN3 |
| PILRA | NELL2 | ABLIM1 | TGIF2 | CD300LB |
| HLA-DRB5 | GBP2 | GIMAP7 | LFNG | CREB5 |
| LTB | ARL4C | CCL3L3 | MFAP3L | DPP4 |
| EPHB1 | RASGRP1 | ZFP36 | ARHGEF40 | SHISA5 |
| CD3D | CD3E | OPTN | MYBL1 | CXCL16 |
| SLC43A2 | SLC7A7 | DUSP1 | CD22 | PRR5 |
| POU2F2 | IGSF6 | DPM2 | STRADB | NFIX |
| LCK | TNFRSF25 | LBH | MS4A1 | SELENBP1 |
| PRKCH | POC1B | KLF2 | BTN3A1 | TMCC3 |
| MBNL3 | CD300E | NRGN | AKAP7 | RP11-599B13.6 |
| GZMM | PTGS1 | MEFV | DYRK2 | DUSP6 |
| BCL11B | NAMPT | MBP | GNG8 | TCP11L2 |
| CXCL1 | PLD4 | NUAK2 | CD79A | EGR1 |
| TBX21 | PTPN4 | CD8A | POLL | LGALS9 |
| FCMR | RCAN3 | HCAR2 | BTG2 | PLK3 |
| BBOF1 | NLRP6 | FOXO4 | NINJ2 | SLFN5 |
| CD14 | PRKAG2 | ARHGAP26 | KLC3 | |
| GZMH | 8-Mar | RGS10 | GBP5 | |
| MPEG1 | TUBA1A | ZAP70 | CARD16 | |
| CLIC3 | S1PR5 | RARRES3 | EMP3 | |
| CYTH4 | LY86 | RASSF5 | TRIM34 | |
| XKR8 | PLEKHF1 | ITK | ZFYVE28 | |
| TNFRSF1B | ESPN | PRDM1 | B4GALT7 | |
| LEF1 | MKRN1 | HLA-DQB1 | NSG1 | |
| PVALB | PF4V1 | VIM-AS1 | KCNA3 | |
| CD247 | MYOM2 | SH2D3C | TFEB | |
| ABI3 | CSRNP1 | CSF2RB | ERGIC1 | |
| BIN1 | BTN3A2 | TRIM58 | SOD2 | |
| SNCA | CCR7 | FCGR2A | THBD | |
| HLA-DPA1 | C15orf39 | TESPA1 | NOTCH1 | |
| DPEP2 | CD8B | LCOR | HCAR3 | |
| PSMF1 | PDLIM2 | PTGER4 | PRSS33 | |
| CPPED1 | IDS | ST6GALNAC2 | NFATC2 | |
| LILRA1 | FBXO7 | CD27 | MYL9 | |
| UBAP2 | LGALS2 | XCL2 | FAXDC2 | |
| CD3G | KLF12 | ITGAL | SDPR | |
| MAP3K7CL | KRT1 | S1PR1 | PPP1R16B | |
| BCL9L | AUTS2 | SPECC1 | TBCEL | |
| | HLA-DRA | | | |

### Example 10

### Non-Invasive Measurement of Bone Marrow-Specific Transcripts by cf-mRNA Profiling in Multiple Myeloma patients

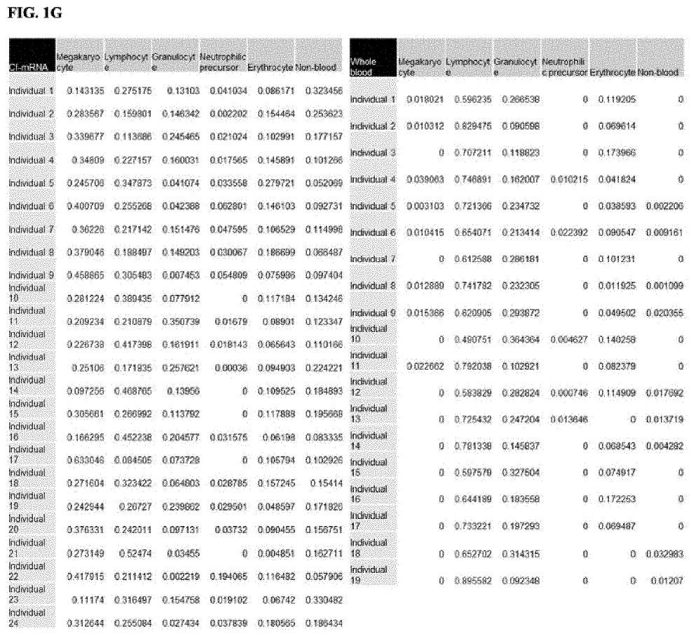

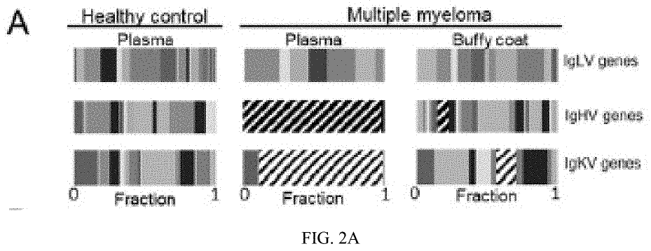

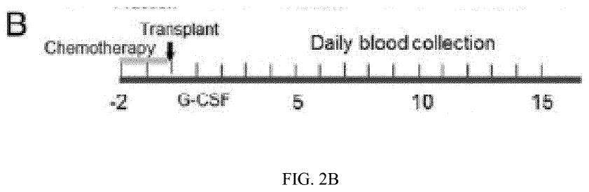

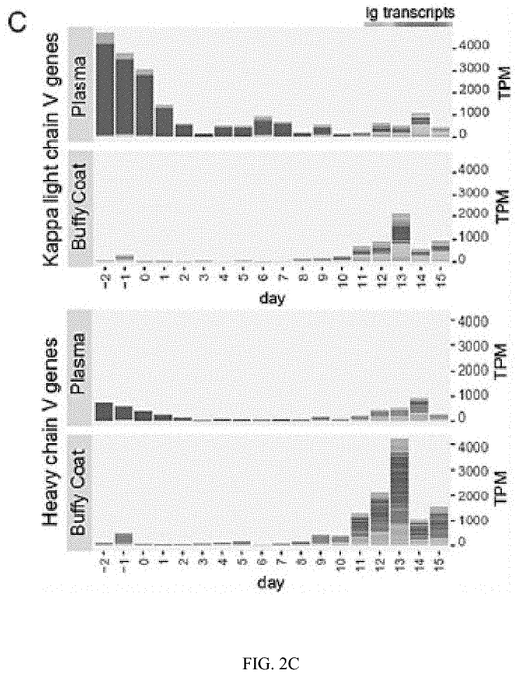

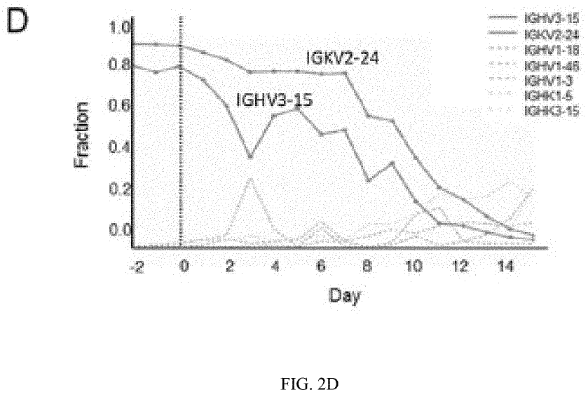

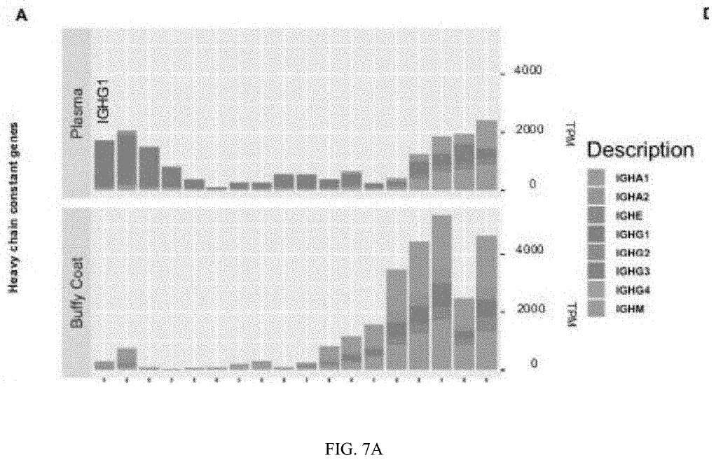

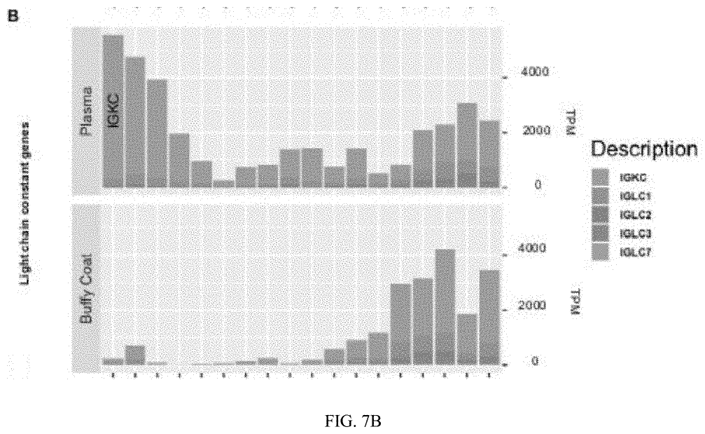

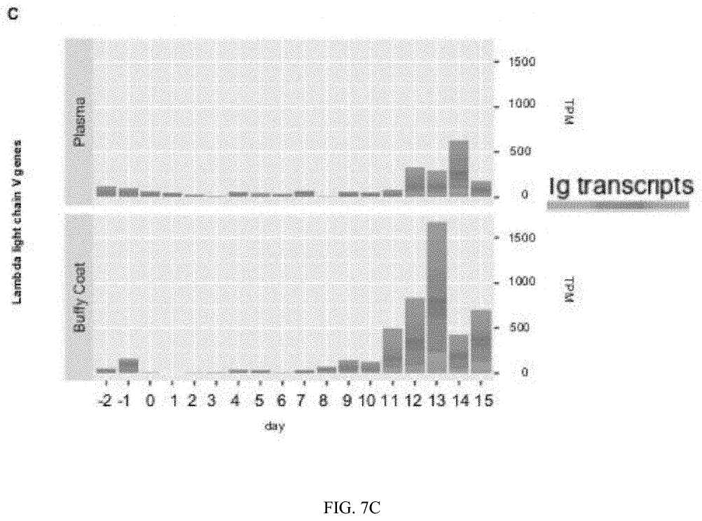

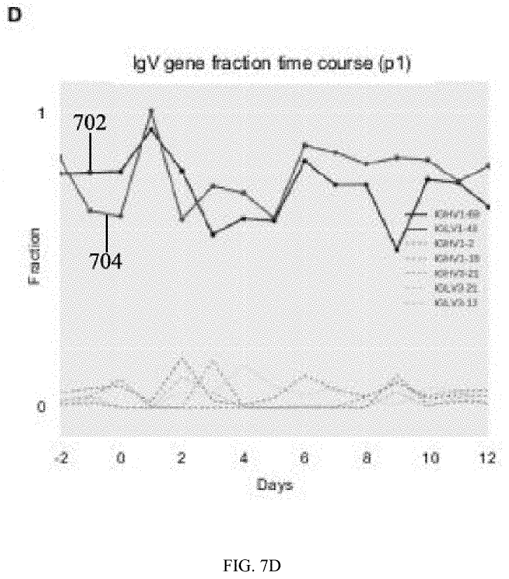

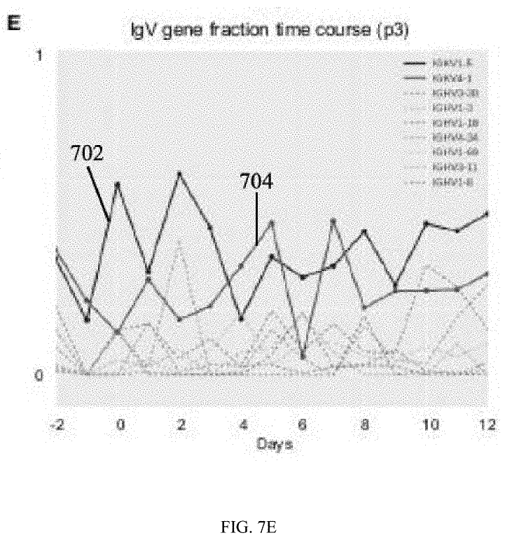

As further evidence that BM-specific transcripts may be detected in cf-mRNA and to evaluate their potential utility, three multiple myeloma (MM) patients were recruited. MM is characterized by the clonal expansion and accumulation of malignant plasma cells almost exclusively in the BM. These cells express specific immunoglobulin (Ig) rearrangements, in contrast to plasma cells of healthy individuals, which express multiple Ig combinations. MM patients underwent melphalan-mediated BM ablation (starting at day −2) followed by autologous hematopoietic stem cell (HSC) infusion (day 0) (FIG. 2B). Cf-mRNA from 1 ml of plasma of these patients before BM ablation (day −2) were isolated and sequenced. Clonal expansion of Ig heavy (IgH) and Ig light (IgL) chains transcripts was identified for two out of three patients. For instance, in Patient 2, IGHG1 and IGKC transcripts as the most prevalent Ig constant regions (FIGS. 7A-7C) were detected. For the variable regions, Ighv3-15 and Igkv2-24 transcripts dominated the sample's transcriptome, while no clonal lambda regions were detected (FIGS. 2A, C and FIG. 7C). In contrast, no clonal transcripts were observed in plasma of a healthy individual, as expected (FIG. 2A). Similar analyses in Patient 1 revealed a clone composed of the IgH constant chain IGHA1 and variable region IGHV1-69, and IgL lambda chain IGL1 and variable region IGLV1-40 (FIG. 7D). In both cases, the malignant clones were consistent with the molecular testing performed from BM aspirates (Table 1). However, for Patient 3, no dominant Ig rearrangements were detected (FIG. 7E), likely due to the low number of plasma cells in the BM of this Patient at the start of this study (Table 1). Malignant plasma cells are rarely found in circulation in MM patients; indeed, RNA-Seq analysis of the matching buffy coat of Patient 2 samples before chemotherapy treatment showed only low levels of a repertoire of IgH and IgL transcripts, with no dominant rearrangements (FIGS. 2A, C, and FIGS. 7A-7C), highlighting the unique ability of cf-mRNA to capture the clonal Ig transcripts generated by plasma cells in the BM.

TABLE 10
Levels (TPM) of Ig transcripts in plasma during BM ablation and reconstitution of MM patient 2 in plasma - Kappa light chain variable genes
Transcripts per million (TPM)

| | IGKV1- | IGKV1- | IGKV1- | IGKV1- | IGKV1- | IGKV1- | IGKV1- | IGKV1- | IGKV1- | IGKV1- | IGKV1- | IGKV1D- | IGKV1D- |
|---|---|---|---|---|---|---|---|---|---|---|---|---|---|
| Day | 12 | 16 | 17 | 27 | 33 | 37 | 39 | 5 | 6 | 8 | 9 | 12 | 13 |
| −2 | 460.8 | 0.0 | 9.2 | 18.0 | 0.0 | 0.0 | 0.0 | 0.0 | 9.1 | 3.6 | 0.0 | 0.0 | 7.3 |
| −1 | 247.1 | 7.5 | 3.7 | 0.0 | 0.0 | 0.0 | 0.0 | 4.0 | 3.7 | 20.1 | 3.2 | 0.0 | 0.0 |
| 0 | 213.3 | 0.0 | 0.0 | 2.5 | 0.0 | 0.0 | 0.0 | 9.4 | 2.5 | 11.8 | 2.2 | 0.0 | 0.0 |
| 1 | 119.7 | 2.3 | 0.0 | 4.4 | 0.0 | 0.0 | 0.0 | 13.1 | 0.0 | 4.8 | 0.0 | 0.0 | 0.0 |
| 2 | 44.8 | 0.0 | 0.0 | 0.0 | 0.0 | 0.0 | 0.0 | 13.0 | 0.0 | 5.0 | 3.5 | 0.0 | 0.0 |
| 3 | 12.9 | 0.9 | 1.7 | 0.8 | 0.0 | 0.0 | 0.0 | 2.6 | 0.0 | 1.6 | 0.7 | 0.0 | 0.0 |
| 4 | 44.1 | 2.4 | 0.0 | 4.7 | 0.0 | 0.0 | 0.0 | 0.0 | 2.4 | 1.2 | 4.1 | 0.0 | 0.0 |
| 5 | 87.1 | 5.2 | 0.0 | 0.0 | 0.0 | 0.0 | 0.0 | 2.8 | 0.0 | 11.3 | 0.0 | 0.0 | 0.0 |
| 6 | 121.6 | 0.0 | 0.0 | 33.8 | 0.0 | 0.0 | 0.0 | 36.2 | 0.0 | 18.5 | 0.0 | 0.0 | 0.0 |
| 7 | 42.3 | 0.0 | 4.6 | 4.5 | 0.0 | 0.0 | 0.0 | 9.8 | 4.6 | 13.9 | 0.0 | 0.0 | 0.0 |
| 8 | 34.2 | 0.0 | 0.0 | 0.0 | 0.0 | 0.0 | 0.0 | 14.5 | 0.0 | 9.6 | 0.0 | 0.0 | 0.0 |
| 9 | 74.9 | 8.9 | 5.9 | 2. [symbol] | 2.1 | 0.0 | 0.0 | 13.9 | 0.0 | 13.0 | 2.5 | 0.0 | 0.0 |
| 10 | 20.7 | 1.0 | 2.1 | 3.1 | 0.0 | 0.0 | 0.0 | 6.1 | 1.0 | 8.3 | 5.3 | 0.0 | 0.0 |
| 11 | 29. [symbol] | 7.7 | 9.4 | 2.5 | 0.8 | 0.0 | 0.0 | 18.4 | 3.4 | 8.6 | 6.2 | 0.0 | 0.0 |
| 12 | 79.2 | 16.8 | 24.5 | 6.8 | 0.0 | 0.0 | 0.0 | 41.3 | 9.1 | 29.2 | 11.7 | 0.0 | 0.0 |
| 13 | 74.8 | 24.9 | 10.2 | 8.6 | 0.0 | 0.0 | 1.8 | 40.4 | 2.8 | 28.5 | 10.7 | 0.0 | 1.2 |
| 14 | 153.6 | 30.5 | 18.3 | 11.9 | 4.0 | 0.0 | 3.6 | [symbol] | 15.6 | 43.0 | 21.5 | 0.0 | 0.0 |
| 15 | [symbol] | 11.6 | 5. [symbol] | 3.4 | 1.1 | 0.0 | 0.0 | 21.3 | 2.3 | 24.1 | 9.5 | 0.0 | 0.0 |

| | IGKV1D- | IGKV1D- | IGKV1D- | IGKV1D- | IGKV1D- | IGKV1D- | IGKV1D- | IGKV1D- | IGKV1OR2- | IGKV2- |
|---|---|---|---|---|---|---|---|---|---|---|
| Day | 16 | 17 | 33 | 37 | 39 | 42 | 43 | [symbol] | 105 | 24 |
| −2 | 0.0 | 0.0 | 0.0 | 0.0 | 9.3 | 0.0 | 0.0 | 0.0 | 0.0 | 4149.3 |
| −1 | 0.0 | 1.0 | 0.0 | 0.0 | 0.0 | 0.0 | 0.0 | 0.0 | 0.0 | 3395.6 |
| 0 | 0.0 | 1.3 | 0.0 | 0.0 | 2.6 | 0.0 | 0.0 | 1.3 | 3.0 | 2750.6 |
| 1 | 0.0 | 1.2 | 0.0 | 0.0 | 0.0 | 0.0 | 1.2 | 0.0 | 0.0 | 1282.1 |
| 2 | 0.0 | 0.0 | 0.0 | 0.0 | 0.0 | 0.0 | 0.0 | 0.0 | 0.0 | 527.3 |
| 3 | 0.0 | 0.0 | 0.0 | 0.0 | 0.0 | 0.0 | 0.0 | 0.0 | 0.0 | 132.4 |
| 4 | 0.0 | 0.0 | 0.0 | 0.0 | 0.0 | 0.0 | 0.0 | 0.0 | 0.0 | 442.9 |
| 5 | 0.0 | 0.0 | 0.0 | 0.0 | 0.0 | 0.0 | 0.0 | 0.0 | 0.0 | 427.1 |
| 6 | 0.0 | 0.0 | 0.0 | 0.0 | 0.0 | 0.0 | 0.0 | 0.0 | 0.0 | 662.9 |
| 7 | 0.0 | 0.0 | 0.0 | 0.0 | 0.0 | 0.0 | 0.0 | 0.0 | 0.0 | 583.0 |
| 8 | 0.0 | 0.0 | 0.0 | 0.0 | 0.0 | 0.0 | 0.0 | 0.0 | 0.0 | 149.7 |
| 9 | 0.0 | 3.1 | 2.1 | 0.0 | 0.0 | 0.0 | 0.0 | 0.0 | 0.0 | 349.6 |
| 10 | 0.0 | 0.0 | 0.0 | 0.0 | 0.0 | 0.0 | 0.6 | 0.0 | 0.0 | 70.3 |
| 11 | 3.4 | 2.3 | 0.0 | 0.0 | 0.9 | 0.0 | 0.0 | 0.4 | 0.0 | 57.9 |
| 12 | 5.9 | 0.9 | 0.0 | 0.0 | 0.0 | 0.0 | 1.0 | 2.0 | 0.0 | 185.6 |
| 13 | 8.7 | 1.1 | 0.0 | 0.0 | 0.0 | 0.0 | 0.4 | 2.6 | 0.0 | 77.7 |
| 14 | 12.2 | 3.3 | 0.0 | 0.0 | 0.0 | 0.0 | 5.1 | 0.0 | 0.0 | 89.2 |
| 15 | 0.0 | 1.2 | 0.0 | 0.0 | 0.0 | 0.0 | 0.0 | 0.0 | 0.0 | 21. [symbol] |

| | IGKV2- | IGKV2- | IGKV2- | IGKV2D- | IGKV2D- | IGKV2D- | IGKV2D- | IGKV2D- | IGKV2D- | IGKV3- | IGKV3- | IGKV3- | IGKV3- |
|---|---|---|---|---|---|---|---|---|---|---|---|---|---|
| Day | 28 | 30 | 40 | 24 | 26 | 28 | 29 | 30 | 40 | 11 | 15 | 20 | 7 |
| −2 | 0.0 | 0.0 | 0.0 | 0.0 | 0.0 | 0.0 | 0.0 | 25.3 | 0.0 | 8.6 | 0.0 | 16.5 | 0.0 |
| −1 | 0.0 | 5.2 | 0.0 | 2.1 | 0.0 | 0.0 | 5.9 | 13.7 | 0.0 | 19.1 | [symbol] | 33.3 | 0.0 |
| 0 | 0.0 | 0.0 | 0.0 | 11.1 | 0.0 | 0.0 | 0.0 | 14.0 | 0.0 | 11.8 | 5.8 | 13.6 | 0.0 |
| 1 | 0.0 | 0.0 | 0.0 | 0.0 | 0.0 | 0.0 | 0.0 | 4.1 | 0.0 | 4.2 | 4.9 | 16.1 | 0.0 |
| 2 | 0.0 | 0.0 | 0.0 | 0.0 | 0.0 | 0.0 | 0.0 | 0.0 | 0.0 | 3.8 | 0.0 | 14.6 | 0.0 |
| 3 | 0.0 | 0.0 | 0.0 | 0.0 | 0.0 | 0.0 | 0.0 | 0.8 | 0.0 | 2.4 | 1.6 | 0.7 | 0.0 |
| 4 | 0.0 | 0.0 | 0.0 | 2.3 | 0.0 | 0.0 | 0.0 | 0.0 | 0.0 | 4.4 | 1.8 | 8.5 | 0.0 |
| 5 | 0.0 | 0.0 | 0.0 | 0.0 | 0.0 | 0.0 | 0.0 | 0.0 | 0.0 | 0.0 | 0.0 | 0.0 | 0.0 |
| 6 | 0.0 | 0.0 | 0.0 | 0.0 | 0.0 | 0.0 | 0.0 | 0.0 | 0.0 | 0.0 | 0.0 | 46.0 | 0.0 |
| 7 | 0.0 | 4.3 | 0.0 | 0.0 | 0.0 | 0.0 | 0.0 | 0.0 | 0.0 | 4.3 | 0.0 | 8.2 | 0.0 |
| 8 | 0.0 | 0.0 | 0.0 | 0.0 | 0.0 | 0.0 | 0.0 | 0.0 | 0.0 | 0.0 | [symbol] | 7.9 | 0.0 |
| 9 | 0.0 | 2.7 | 0.0 | 0.0 | 0.0 | 0.0 | 0.0 | 5.4 | 0.0 | 5.5 | 0.0 | 47.4 | 0.0 |
| 10 | 0.0 | 1.0 | 0.0 | 0.0 | 0.0 | 0.0 | 0.0 | 1.0 | 0.0 | 4.8 | 0.8 | 13.9 | 0.0 |
| 11 | 0.0 | 3.2 | 0.0 | 0.0 | 0.0 | 0.0 | 0.8 | 3.1 | 0.0 | 11.8 | 2.4 | 27. [symbol] | 0.0 |
| 12 | 0.0 | 16.4 | 0.0 | 0.0 | 0.0 | 0.0 | 0.0 | 2.7 | 0.0 | 21.9 | 18.3 | 103.6 | 1.0 |
| 13 | 0.0 | 19.1 | 0.0 | 0.0 | 0.0 | 0.0 | 4.1 | 0.0 | 0.0 | 23.0 | 25. [symbol] | 67.8 | 0.5 |
| 14 | 0.0 | 13.2 | 0.0 | 0.0 | 0.0 | 0.0 | 1.9 | 7.4 | 0.0 | 95.6 | 180.1 | 183.0 | 0.0 |
| 15 | 0.0 | 3.2 | 0.0 | 0.0 | 0.0 | 0.0 | 0.0 | 2.1 | 0.0 | 12.9 | 143.2 | 52.7 | 0.0 |

| | IGKV3D- | IGKV3D- | IGKV3D- | IGKV3D- | IGKV3OR2- | IGKV4- | IGKV5- | IGKV6- | IGKV6D- | IGKV6D- |
|---|---|---|---|---|---|---|---|---|---|---|
| Day | 11 | 15 | 20 | 7 | 268 | 1 | 2 | 21 | 21 | 41 |
| −2 | 0.0 | 0.0 | 0.0 | 0.0 | 0.0 | 19. [symbol] | 0.0 | 0.0 | 0.0 | 0.0 |
| −1 | 0.0 | 0.0 | 2.7 | 0.0 | 0.0 | 22.4 | 8.0 | 0.0 | 0.0 | 0.0 |
| 0 | 0.0 | 0.0 | 0.0 | 0.0 | 0.0 | 10.6 | 2.2 | 0.0 | 0.0 | 0.0 |
| 1 | 0.0 | 0.0 | 0.0 | 0.0 | 0.0 | 7.0 | 0.0 | 2.0 | 0.0 | 0.0 |
| 2 | 0.0 | 0.0 | 0.0 | 0.0 | 0.0 | 5.4 | 0.0 | 0.0 | 0.0 | 0.0 |
| 3 | 0.0 | 0.0 | 0.6 | 0.0 | 0.0 | 3.8 | 0.0 | 1. [symbol] | 0.0 | 0.0 |
| 4 | 0.0 | 0.0 | 0.0 | 0.0 | 0.0 | 10.0 | 2.1 | 0.0 | 0.0 | 0.0 |
| 5 | 0.0 | 0.0 | 0.0 | 0.0 | 0.0 | 21.6 | 0.0 | 0.0 | 0.0 | 0.0 |
| 6 | 0.0 | 0.0 | 0.0 | 0.0 | 0.0 | 36.4 | 0.0 | 0.0 | 0.0 | 0.0 |
| 7 | 0.0 | 0.0 | 0.0 | 0.0 | 0.0 | 16.9 | 0.0 | 0.0 | 0.0 | 0.0 |
| 8 | 0.0 | 0.0 | 0.0 | 0.0 | 0.0 | 14.2 | 0.0 | 0.0 | 0.0 | 0.0 |
| 9 | 0.0 | 0.0 | 0.0 | 0.0 | 0.0 | 34.9 | 2.6 | 0.0 | 0.0 | 0.0 |
| 10 | 1.5 | 0.0 | 0.0 | 0.0 | 0.0 | 13.0 | 0.0 | 1.5 | 0.0 | 0.0 |
| 11 | 0.0 | 0.0 | 0. [symbol] | 0.0 | 0.0 | 19.3 | 3.6 | 2.2 | 0.0 | 0.0 |
| 12 | 1.8 | 0.0 | 1.4 | 1.0 | 0.0 | 69.2 | 2.5 | 3.4 | 0.6 | 0.0 |
| 13 | 2.1 | 0.0 | 2.1 | 0.0 | 0.0 | [symbol] | 3.7 | 4.4 | 0.0 | 0.0 |
| 14 | 4.3 | 1.9 | 2.9 | 0.0 | 0.0 | 95.7 | 1.7 | 8.7 | 1.7 | 0.0 |
| 15 | 2.3 | 7.2 | 1.5 | 0.0 | 0.0 | 39.5 | 2.0 | 2.0 | 1.0 | 0.0 |

[symbol]

indicates data missing or illegible when filed

TABLE 11
Levels (TPM) of Ig transcripts in plasma during BM ablation and
reconstitution of MM patient 2 in plasma - heavy chain variable genes
Transcripts per million (TPM)

| Day | IGHV6-1 | IGHV1-2 | IGHV1-3 | IGHV2-5 | IGHV3-7 | IGHV3-11 | IGHV3-13 | IGHV3-15 | IGHV3-16 |
|---|---|---|---|---|---|---|---|---|---|
| −2 | 0.0 | 0.0 | 0.0 | 0.0 | 0.0 | 0.0 | 0.0 | 715.4 | 0.0 |
| −1 | 0.0 | 0.0 | 10.3 | 1.9 | 2.9 | 2.4 | 0.0 | 516.1 | 0.0 |
| 0 | 0.0 | 0.0 | 9.3 | 0.0 | 0.0 | 0.0 | 0.0 | 377.4 | 0.0 |
| 1 | 0.0 | 1.6 | 18.5 | 0.0 | 3.5 | 0.0 | 0.0 | 210.8 | 0.0 |
| 2 | 0.0 | 0.0 | 3.7 | 0.0 | 0.0 | 0.0 | 3.2 | 89.1 | 0.0 |
| 3 | 0.0 | 0.0 | 28.6 | 0.0 | 0.0 | 0.0 | 0.0 | 11.3 | 0.0 |
| 4 | 0.0 | 0.0 | 6.6 | 0.0 | 1.9 | 0.0 | 0.0 | 46.7 | 0.0 |
| 5 | 0.0 | 0.0 | 0.0 | 0.0 | 4.0 | 0.0 | 0.0 | 47.0 | 0.0 |
| 6 | 0.0 | 0.0 | 0.0 | 0.0 | 0.0 | 0.0 | 0.0 | 25.7 | 0.0 |
| 7 | 0.0 | 7.6 | 0.0 | 0.0 | 3.6 | 0.0 | 0.0 | 52.0 | 0.0 |
| 8 | 0.0 | 7.3 | 0.0 | 0.0 | 6.9 | 0.0 | 0.0 | 13.4 | 0.0 |
| 9 | 0.0 | 2.4 | 5.4 | 5.9 | 4.6 | 3.8 | 0.0 | 41.9 | 0.0 |
| 10 | 0.9 | 1.7 | 14.3 | 0.0 | 4.0 | 0.0 | 0.8 | 6.6 | 0.0 |
| 11 | 1.4 | 6.2 | 40.9 | 2.6 | 19.9 | 3.7 | 1.9 | 10.0 | 0.0 |
| 12 | 4.8 | 8.8 | 10.7 | 5.9 | 26.7 | 6.2 | 2.2 | 22.4 | 0.0 |
| 13 | 7.8 | 29.2 | 6.0 | 3.7 | 42.8 | 5.1 | 0.6 | 24.2 | 0.0 |
| 14 | 8.3 | 24.6 | 5.5 | 30.5 | 49.2 | 10.2 | 0.0 | 28.3 | 1.6 |
| 15 | 8.6 | 10.4 | 5.3 | 6.9 | 20.5 | 3.0 | 0.9 | 7.8 | 0.0 |

| Day | IGHV1-18 | IGHV3-20 | IGHV3-21 | IGHV3-23 | IGHV1-24 | IGHV2-26 | IGHV4-28 | IGHV3-33 |
|---|---|---|---|---|---|---|---|---|
| −2 | 0.0 | 0.0 | 0.0 | 0.0 | 0.0 | 0.0 | 0.0 | 0.0 |
| −1 | 11.1 | 1.5 | 5.8 | 1.4 | 0.0 | 0.0 | 1.5 | 0.0 |
| 0 | 0.0 | 2.1 | 0.0 | 1.9 | 0.0 | 0.0 | 0.0 | 2.0 |
| 1 | 1.9 | 0.0 | 3.5 | 1.7 | 0.0 | 0.0 | 0.0 | 0.0 |
| 2 | 7.0 | 0.0 | 6.3 | 9.4 | 3.5 | 0.0 | 0.0 | 0.0 |
| 3 | 0.7 | 0.0 | 0.0 | 0.6 | 0.0 | 0.0 | 0.0 | 0.0 |
| 4 | 2.0 | 0.0 | 0.0 | 1.8 | 0.0 | 0.0 | 0.0 | 1.9 |
| 5 | 0.0 | 0.0 | 0.0 | 0.0 | 0.0 | 0.0 | 0.0 | 0.0 |
| 6 | 14.6 | 0.0 | 0.0 | 0.0 | 0.0 | 0.0 | 0.0 | 0.0 |
| 7 | 0.0 | 0.0 | 0.0 | 0.0 | 0.0 | 0.0 | 0.0 | 0.0 |
| 8 | 0.0 | 0.0 | 0.0 | 0.0 | 0.0 | 0.0 | 0.0 | 13.8 |
| 9 | 12.5 | 0.0 | 6.8 | 9.0 | 2.5 | 5.8 | 2.3 | 2.2 |
| 10 | 5.3 | 0.9 | 0.8 | 4.0 | 0.0 | 0.0 | 1.6 | 0.0 |
| 11 | 14.2 | 1.4 | 5.8 | 7.6 | 1.4 | 3.3 | 0.9 | 7.0 |
| 12 | 40.4 | 3.2 | 15.9 | 20.0 | 3.3 | 5.8 | 4.1 | 34.2 |
| 13 | 55.8 | 0.6 | 15.0 | 21.5 | 3.7 | 16.5 | 3.5 | 14.9 |
| 14 | 83.3 | 4.4 | 30.7 | 44.2 | 10.2 | 89.9 | 12.6 | 16.8 |
| 15 | 45.0 | 0.0 | 10.7 | 7.9 | 0.0 | 19.3 | 0.9 | 3.5 |

| Day | IGHV4-34 | IGHV3-35 | IGHV3-38 | IGHV4-39 | IGHV1-45 | IGHV1-46 | IGHV3-48 | IGHV3-49 | IGHV5-51 |
|---|---|---|---|---|---|---|---|---|---|
| −2 | 0.0 | 0.0 | 0.0 | 0.0 | 0.0 | 0.0 | 0.0 | 0.0 | 15.7 |
| −1 | 20.0 | 0.0 | 0.0 | 3.0 | 0.0 | 0.7 | 1.4 | 1.4 | 6.3 |
| 0 | 2.3 | 2.7 | 0.0 | 0.0 | 0.0 | 0.0 | 0.0 | 0.0 | 2.2 |
| 1 | 2.0 | 0.0 | 0.0 | 1.8 | 0.0 | 1.7 | 0.0 | 5.7 | 11.5 |
| 2 | 11.0 | 0.0 | 0.0 | 3.3 | 0.0 | 0.0 | 0.0 | 0.0 | 3.5 |
| 3 | 1.5 | 0.0 | 0.0 | 0.0 | 0.0 | 0.0 | 0.0 | 0.0 | 0.7 |
| 4 | 8.5 | 0.0 | 0.0 | 1.9 | 2.0 | 0.0 | 1.8 | 0.0 | 0.0 |
| 5 | 4.7 | 0.0 | 0.0 | 4.1 | 0.0 | 0.0 | 4.0 | 0.0 | 0.0 |
| 6 | 0.0 | 0.0 | 0.0 | 13.6 | 0.0 | 0.0 | 0.0 | 0.0 | 0.0 |
| 7 | 4.1 | 0.0 | 0.0 | 0.0 | 0.0 | 1.8 | 0.0 | 0.0 | 0.0 |
| 8 | 0.0 | 9.4 | 0.0 | 0.0 | 0.0 | 0.0 | 0.0 | 0.0 | 7.6 |
| 9 | 5.3 | 0.0 | 0.0 | 2.3 | 0.0 | 1.1 | 4.5 | 3.7 | 15.0 |
| 10 | 9.3 | 0.0 | 0.0 | 5.8 | 0.0 | 2.0 | 2.4 | 0.0 | 2.6 |
| 11 | 26.3 | 0.0 | 0.0 | 7.4 | 0.0 | 0.3 | 8.9 | 5.0 | 2.1 |
| 12 | 45.3 | 0.0 | 0.0 | 26.0 | 0.8 | 4.2 | 11.8 | 12.9 | 19.0 |
| 13 | 71. [symbol] | 0.0 | 0.0 | 27.1 | 0.0 | 4.9 | 20.4 | 15.5 | 20.2 |
| 14 | 91.5 | 0.0 | 0.0 | 80.4 | 0.0 | 38.7 | 27.4 | 9.8 | 23.8 |
| 15 | 31.9 | 0.0 | 0.0 | 8.2 | 0.0 | 11.9 | 12.4 | 3.3 | 10.8 |

| Day | IGHV3-53 | IGHV1-58 | IGHV4-61 | IGHV3-66 | IGHV1-69 | IGHV2-70 | IGHV3-73 | IGHV7-81 |
|---|---|---|---|---|---|---|---|---|
| −2 | 0.0 | 0.0 | 0.0 | 0.0 | 0.0 | 0.0 | 0.0 | 0.0 |
| −1 | 1.8 | 1.6 | 0.0 | 0.0 | 0.0 | 1.4 | 0.0 | 0.0 |
| 0 | 0.0 | 0.0 | 0.0 | 0.0 | 0.0 | 0.0 | 0.0 | 0.0 |
| 1 | 0.0 | 1.9 | 0.0 | 0.0 | 0.0 | 1.7 | 0.0 | 0.0 |
| 2 | 0.0 | 0.0 | 2.8 | 0.0 | 0.0 | 0.0 | 0.0 | 0.0 |
| 3 | 0.0 | 0.0 | 0.0 | 0.0 | 0.0 | 0.0 | 0.0 | 0.0 |
| 4 | 0.0 | 0.0 | 0.0 | 0.0 | 0.0 | 0.0 | 0.0 | 0.0 |
| 5 | 0.0 | 0.0 | 0.0 | 0.0 | 0.0 | 0.0 | 0.0 | 0.0 |
| 6 | 0.0 | 0.0 | 0.0 | 0.0 | 0.0 | 0.0 | 0.0 | 0.0 |
| 7 | 0.0 | 0.0 | 0.0 | 0.0 | 0.0 | 0.0 | 0.0 | 0.0 |
| 8 | 0.0 | 0.0 | 0.0 | 0.0 | 0.0 | 0.0 | 0.0 | 0.0 |
| 9 | 0.0 | 0.0 | 0.0 | 0.0 | 5.0 | 0.0 | 0.0 | 0.0 |
| 10 | 0.5 | 0.0 | 0.7 | 0.8 | 0.0 | 0.0 | 0.8 | 0.0 |
| 11 | 1.1 | 0.0 | 0.5 | 0.7 | 2.1 | 0.0 | 0.0 | 0.0 |
| 12 | 3.5 | 1.6 | 1.4 | 1.5 | 1.6 | 2.2 | 0.7 | 0.0 |
| 13 | 5.7 | 2.4 | 1.6 | 0.0 | 1.2 | 2.3 | 2.7 | 0.0 |
| 14 | 5.4 | 1.7 | 1.5 | 0.0 | 10.1 | 13.4 | 1.5 | 0.0 |
| 15 | 2.7 | 0.0 | 2.2 | 0.0 | 2.9 | 1.7 | 1.7 | 0.0 |

[symbol]

indicates data missing or illegible when filed

TABLE 12
Levels (TPM) of Ig transcripts in plasma during BM
ablation and reconstitution of MM patient 2 in plasma -
heavy chain and light chain constant genes
Heavy chain constant genes
Transcripts per million (TPM)

| Day | IGHA1 | IGHA2 | IGHE | IGHG1 | IGHG2 | IGHG3 | IGHG4 |
|---|---|---|---|---|---|---|---|
| −2 | 44.1 | 4.1 | 1.4 | 1557.1 | 16.7 | 27.0 | 36.4 |
| −1 | 91.3 | 51.3 | 0.4 | 1663.6 | 35.6 | 42.5 | 49.8 |
| 0 | 38.1 | 1.5 | 1.4 | 1331.1 | 16.7 | 28.2 | 36.4 |
| 1 | 22.7 | 2.2 | 0.0 | 672.3 | 15.1 | 13.6 | 17.3 |
| 2 | 30.4 | 4.0 | 0.0 | 289.9 | 5.9 | 11.7 | 6.1 |
| 3 | 13.6 | 1.0 | 0.5 | 58.6 | 4.0 | 1.8 | 2.5 |
| 4 | 16.7 | 2.8 | 1.3 | 176.0 | 7.8 | 5.5 | 8.8 |
| 5 | 20.4 | 0.0 | 0.0 | 197.3 | 7.7 | 4.7 | 11.6 |
| 6 | 28.2 | 3.3 | 0.0 | 405.4 | 40.4 | 13.4 | 18.7 |
| 7 | 27.2 | 6.4 | 0.0 | 423.3 | 15.4 | 14.5 | 11.5 |
| 8 | 28.0 | 5.5 | 2.6 | 264.3 | 32.3 | 7.0 | 8.5 |
| 9 | 83.3 | 22.9 | 0.4 | 360.2 | 53.0 | 11.5 | 20.2 |
| 10 | 38.4 | 5.9 | 0.4 | 113.9 | 22.2 | 6.0 | 5.1 |
| 11 | 98.4 | 17.1 | 0.4 | 136.0 | 34.5 | 7.3 | 6.5 |
| 12 | 236.5 | 33.0 | 1.3 | 468.3 | 98.6 | 22.5 | 24.1 |
| 13 | 556.2 | 86.9 | 1.2 | 436.4 | 143.7 | 23.7 | 20.5 |
| 14 | 305.9 | 51.6 | 3.0 | 645.2 | 217.8 | 36.4 | 41.1 |
| 15 | 938.5 | 57.6 | 3.6 | 326.5 | 177.6 | 19.5 | 17.5 |

Light chain constant genes
Transcripts per million (TPM)

| Day | IGKC | IGLC1 | IGLC2 | IGLC3 | IGLC7 |
|---|---|---|---|---|---|
| −2 | 5258.0 | 247.8 | 31.9 | 31.8 | 0.0 |
| −1 | 4290.6 | 373.0 | 44.9 | 57.6 | 1.4 |
| 0 | 3587.2 | 294.3 | 30.9 | 32.6 | 5.6 |
| 1 | 1755.8 | 167.6 | 16.7 | 22.8 | 3.3 |
| 2 | 797.5 | 112.2 | 26.6 | 23.2 | 0.0 |
| 3 | 212.7 | 29.5 | 4.5 | 7.4 | 1.2 |
| 4 | 664.3 | 50.0 | 11.3 | 16.2 | 0.0 |
| 5 | 708.9 | 85.3 | 24.7 | 21.2 | 0.0 |
| 6 | 997.3 | 270.7 | 46.3 | 58.0 | 0.0 |
| 7 | 1245.9 | 187.2 | 9.4 | 9.4 | 0.0 |
| 8 | 575.4 | 116.7 | 30.5 | 24.3 | 0.0 |
| 9 | 1091.6 | 218.4 | 47.7 | 71.5 | 0.0 |
| 10 | 374.1 | 114.6 | 7.7 | 28.2 | 0.8 |
| 11 | 529.0 | 200.7 | 36.6 | 61.8 | 1.2 |
| 12 | 1439.1 | 383.7 | 127.6 | 131.7 | 2.8 |
| 13 | 1380.9 | 606.2 | 126.8 | 186.1 | 1.6 |
| 14 | 2097.1 | 480.6 | 268.1 | 239.7 | 5.8 |
| 15 | 1689.2 | 518.8 | 75.1 | 140.7 | 1.7 |

TABLE 13
Levels (TPM) of Ig transcripts in plasma during BM ablation and
reconstitution of MM patient 2 in plasma - lambda light chain variable genes
Lambda light chain variable genes
Transcripts per million (TPM)

| Day | IGLV4-69 | IGLV [symbol] -61 | IGLV4- [symbol] 0 | IGLV6-57 | IGLV11-55 | IGLV10-54 | IGLV [symbol] -52 | IGLV1-51 | IGLV1-50 | IGLV [symbol] -4 [symbol] |
|---|---|---|---|---|---|---|---|---|---|---|
| −2 | 0.0 | 7.7 | 0.0 | 0.0 | 0.0 | 0.0 | 0.0 | 8.2 | 0.0 | 0.0 |
| −1 | 1.5 | 1.6 | 0.0 | 1.5 | 0.0 | 0.0 | 0.0 | 8.3 | 0.0 | 0.0 |
| 0 | 0.0 | 4.2 | 0.0 | 0.0 | 0.0 | 0.0 | 0.0 | 8.7 | 0.0 | 0.0 |
| 1 | 0.0 | 0.0 | 0.0 | 2.8 | 0.0 | 0.0 | 1.0 | 0.0 | 0.0 | 0.0 |
| 2 | 3.3 | 0.0 | 0.0 | 6.7 | 0.0 | 0.0 | 0.0 | 0.0 | 0.0 | 0.0 |
| 3 | 0.0 | 0.7 | 0.0 | 0.0 | 0.0 | 0.0 | 0.0 | 0.7 | 0.0 | 0.0 |
| 4 | 1.9 | 4.0 | 0.0 | 2.0 | 0.0 | 0.0 | 0.0 | 8.5 | 0.0 | 0.0 |
| 5 | 0.0 | 0.0 | 0.0 | 2.1 | 0.0 | 0.0 | 0.0 | 0.0 | 0.0 | 0.0 |
| 6 | 0.0 | 0.0 | 0.0 | 0.0 | 0.0 | 0.0 | 17.2 | 0.0 | 0.0 | 0.0 |
| 7 | 0.0 | 3.8 | 0.0 | 5.8 | 0.0 | 0.0 | 0.0 | 4.1 | 0.0 | 0.0 |
| 8 | 0.0 | 0.0 | 0.0 | 0.0 | 0.0 | 0.0 | 0.0 | 0.0 | 0.0 | 0.0 |
| 9 | 0.0 | 0.0 | 0.0 | 1.2 | 0.0 | 0.0 | 0.0 | 0.0 | 0.0 | 0.0 |
| 10 | 0.8 | 4.3 | 0.0 | 0.0 | 0.0 | 0.0 | 0. [symbol] | 0.9 | 0.0 | 0.0 |
| 11 | 2.7 | 4.2 | 0.0 | 0.7 | 0.0 | 0.9 | 0.0 | 1.4 | 0.0 | 0.0 |
| 12 | 18.9 | 9.7 | 0.0 | 2.7 | 0.0 | 4.3 | 0.0 | 13. [symbol] | 0.6 | 1.0 |
| 13 | 5.3 | 10.2 | 3.2 | 4.1 | 0.0 | 6.3 | 0.0 | 12. [symbol] | 0.0 | 0.0 |
| 14 | 2 [symbol] .0 | 21.7 | 0.0 | 7. [symbol] | 0.0 | 2.2 | 1.3 | 37.5 | 0.0 | 0.0 |
| 15 | 7.5 | 5.8 | 1.3 | 3.3 | 0.0 | 2.5 | 0.0 | [symbol] | 0.0 | 0.0 |

| Day | IGLV1-47 | IGLV7-46 | IGLV5-45 | IGLV1-44 | IGLV7-43 | IGLV1-40 | IGLV5-37 | IGLV1-36 |
|---|---|---|---|---|---|---|---|---|
| −2 | 0.0 | 0.0 | 0.0 | 31.3 | 17.7 | 0.0 | 0.0 | 0.0 |
| −1 | 6.4 | 0.0 | 1.7 | 3.7 | 1. [symbol] | 9.7 | 1.9 | 0.0 |
| 0 | 4.4 | 2.5 | 0.0 | 1.7 | 2.5 | 10.9 | 0.0 | 0.0 |
| 1 | 1.9 | 0.0 | 0.0 | 0.0 | 0.0 | 3.9 | 0.0 | 2.1 |
| 2 | 0.0 | 0.0 | 0.0 | 2.7 | 0.0 | 3.5 | 0.0 | 0.0 |
| 3 | 0.0 | 0.0 | 0.0 | 1.7 | 0.0 | 1.5 | 0.0 | 0.0 |
| 4 | 4.1 | 0.0 | 0.0 | 1.6 | 4.6 | 4.1 | 2.5 | 0.0 |
| 5 | 0.0 | 0.0 | 0.0 | 0.0 | 5.0 | 22.5 | 0.0 | 0.0 |
| 6 | 0.0 | 0.0 | 0.0 | 0.0 | 0.0 | 0.0 | 0.0 | 0.0 |
| 7 | 4.0 | 0.0 | 0.0 | 3.1 | 0.0 | 4.0 | 0.0 | 0.0 |
| 8 | 0.0 | 0.0 | 0.0 | 0.0 | 0.0 | 0.0 | 0.0 | 0.0 |
| 9 | 0.0 | 5.7 | 0.0 | 0.0 | 0.0 | 25.4 | 0.0 | 0.0 |
| 10 | 0.9 | 2.0 | 0.9 | 2. [symbol] | 1.0 | 15.2 | 0.0 | 0.0 |
| 11 | 8.0 | 3.3 | 0.8 | 2.7 | 0.8 | 17.3 | 0.0 | 0.0 |
| 12 | 15.1 | 12.3 | 0.0 | 12.7 | 6.6 | 53.9 | 1.0 | 3.6 |
| 13 | 13.7 | 9.8 | 2.6 | 24.1 | 11.2 | 53.6 | 1.5 | 3.4 |
| 14 | 43.2 | 29.3 | 1. [symbol] | 31.2 | 5.9 | 43.2 | 0.0 | 1.9 |
| 15 | 12.9 | 2.2 | 2.1 | 8.4 | 3.3 | 19.9 | 0.0 | 0.0 |

| Day | IGLV2-33 | IGLV3-32 | IGLV3-27 | IGLV3-25 | IGLV2-23 | IGLV3-22 | IGLV3-21 | IGLV3-19 | IGLV2-18 |
|---|---|---|---|---|---|---|---|---|---|
| −2 | 0.0 | 0.0 | 0.0 | 0.0 | 0.0 | 0.0 | 0.0 | 0.0 | 0.0 |
| −1 | 0.0 | 0.0 | 1.9 | 0.0 | 0.0 | 0.0 | 14.7 | 3.8 | 0.0 |
| 0 | 0.0 | 0.0 | 0.0 | 2.5 | 3.0 | 0.0 | 1.1 | 2.6 | 0.0 |
| 1 | 0.0 | 0.0 | 2.3 | 2.2 | 2.6 | 0.0 | 1.0 | 6.8 | 0.0 |
| 2 | 0.0 | 0.0 | 0.0 | 0.0 | 2.4 | 0.0 | 0.0 | 0.0 | 0.0 |
| 3 | 0.0 | 0.0 | 0.0 | 0.0 | 0.0 | 0.0 | 0.0 | 0.9 | 0.0 |
| 4 | 0.0 | 0.0 | 0.0 | 2.4 | 2.8 | 0.0 | 1.0 | 0.0 | 0.0 |
| 5 | 0.0 | 0.0 | 0.0 | 0.0 | 0.0 | 0.0 | 2.3 | 0.0 | 0.0 |
| 6 | 0.0 | 0.0 | 0.0 | 0.0 | 0.0 | 0.0 | 0.0 | 17.4 | 0.0 |
| 7 | 0.0 | 0.0 | 0.0 | 4. [symbol] | 0.0 | 3.9 | 4.0 | 9.3 | 0.0 |
| 8 | 0.0 | 0.0 | 0.0 | 0.0 | 5.3 | 0.0 | 0.0 | 0.0 | 0.0 |
| 9 | 0.0 | 0.0 | 0.0 | 0.0 | 5.1 | 2.5 | 2.5 | 3.0 | 0.0 |
| 10 | 0.0 | 0.0 | 0.0 | 1.0 | 1.2 | 0.0 | 4.1 | 2.1 | 0.0 |
| 11 | 0.0 | 0.0 | 0.0 | 0.8 | 0.9 | 0.7 | 3.8 | 2.6 | 0.0 |
| 12 | 0.0 | 0.0 | 1.0 | 5.8 | 8. [symbol] | 0.0 | 13.7 | 13.9 | 0.0 |
| 13 | 0.0 | 0.0 | 0.7 | 5.1 | 10.0 | 0.0 | 14.1 | 14.7 | 0.8 |
| 14 | 0.0 | 0.0 | 4.1 | 12.1 | 23.7 | 0.0 | 32.9 | 30.7 | 0.0 |
| 15 | 0.0 | 0.0 | 0.0 | 2.3 | 6.7 | 0.0 | 5.0 | 24.4 | 0.0 |

| Day | IGLV3-16 | IGLV2-14 | IGLV3-12 | IGLV2-11 | IGLV3-10 | IGLV3-9 | IGLV4-3 | IGLV3-1 |
|---|---|---|---|---|---|---|---|---|
| −2 | 0.0 | 16.3 | 0.0 | 16.5 | 0.0 | 0.0 | 0.0 | 25.0 |
| −1 | 3.8 | 16.5 | 0.0 | 1.7 | 1.8 | 0.0 | 0.0 | 13.5 |
| 0 | 0.0 | 9.0 | 0.0 | 2.3 | 2.4 | 0.0 | 0.0 | 11.4 |
| 1 | 0.0 | 4.0 | 0.0 | 0.0 | 0.0 | 0.0 | 0.0 | 12.2 |
| 2 | 0.0 | 3. [symbol] | 0.0 | 0.0 | 0.0 | 0.0 | 0.0 | 3.7 |
| 3 | 0.0 | 1.5 | 0.0 | 1.5 | 0.0 | 0.0 | 0.0 | 1.5 |
| 4 | 0.0 | 4.2 | 0.0 | 2.1 | 0.0 | 0.0 | 0.0 | 8.6 |
| 5 | 0.0 | 4.5 | 0.0 | 4.7 | 0.0 | 0.0 | 0.0 | 0.0 |
| 6 | 0.0 | 0.0 | 0.0 | 0.0 | 0.0 | 0.0 | 0.0 | 0.0 |
| 7 | 0.0 | 8.1 | 0.0 | 4.1 | 0.0 | 0.0 | 0.0 | 16.6 |
| 8 | 0.0 | 0.0 | 0.0 | 0.0 | 0.0 | 0.0 | 0.0 | 0.0 |
| 9 | 0.0 | 2. [symbol] | 0.0 | 0.0 | 0.0 | 0.0 | 0.0 | 13.3 |
| 10 | 0.0 | 0.9 | 0.0 | 2.8 | 0.0 | 0.7 | 0.0 | 9.4 |
| 11 | 0.0 | 8.9 | 1.0 | 7.5 | 0.8 | 2.2 | 0.0 | 9.1 |
| 12 | 0.0 | 67.2 | 0.0 | 12.2 | 4.7 | 3.2 | 0.0 | 34.3 |
| 13 | 1.5 | 25. [symbol] | 0.8 | 11. [symbol] | 4.2 | 4.9 | 0.0 | 32.6 |
| 14 | 0.0 | 22.4 | 0.0 | 59.2 | 13.6 | 9.3 | 0.0 | 45.4 |
| 15 | 0.0 | 21.4 | 0.0 | 10.3 | 2.2 | 3.1 | 1.0 | 23.9 |

[symbol]

indicates data missing or illegible when filed

TABLE 14
Levels (TPM) of Ig transcripts in plasma during BM
ablation and reconstitution of MM patient 2 in buffy
coat - heavy chain and light chain constant genes
Heavy chain constant
Transcripts per million (TPM)

| Day | IGHA1 | IGHA2 | IGHE | IGHG1 | IGHG2 | IGHG3 | IGHG4 |
|---|---|---|---|---|---|---|---|
| −2 | 306.0 | 21 [symbol] .4 | 0.0 | 64.9 | 51.1 | 1. [symbol] | 9.8 |
| −1 | 164.9 | 68.7 | 0.0 | 27. [symbol] | 11.5 | 1.5 | 2.9 |
| 0 | 38.7 | 15.7 | 0.0 | 11.3 | 1.9 | 0.4 | 0.5 |
| 1 | 22.2 | 4.1 | 0.5 | 1.9 | 0.0 | 0.6 | 0.0 |
| 2 | 4.5 | 6.5 | 0.0 | 1.2 | 1.1 | 0.0 | 0.0 |
| 3 | 13.3 | 0.6 | 0.0 | 0.6 | 2.2 | 0.0 | 0.0 |
| 4 | 38.6 | 2.4 | 0.6 | 16.6 | 4.2 | 1.0 | 0.8 |
| 5 | 7.7 | 60.6 | 0.0 | 6.7 | 0.0 | 0.3 | 0.0 |
| 6 | 99.1 | 21.5 | 0.5 | 51.8 | 3.7 | 1.3 | 1.0 |
| 7 | 404.5 | 117.5 | 0.2 | 75.8 | 61.1 | 3.4 | 9.9 |
| 8 | 525.3 | 109.8 | 0.0 | 178.7 | 77.3 | 5.7 | 6.0 |
| 9 | 4.4 | 2.9 | 1.2 | 5.4 | 0.5 | 0.0 | 0.0 |
| 10 | 690.1 | 162.7 | 1.0 | 226.4 | 39.0 | 14.1 | 5.9 |
| 11 | 1437.3 | 390.9 | 0.0 | 510.3 | 216.8 | 36.4 | 27.5 |
| 12 | 1618.6 | 409. [symbol] | 3.4 | 633.8 | 279. [symbol] | 35.9 | 17.6 |
| 13 | 1860.8 | 495.6 | 1.5 | 826.2 | 381.2 | 41.2 | 29.9 |
| 14 | 931.7 | 212.1 | 0.4 | 281.8 | 179.8 | 20.6 | 15.2 |
| 15 | 1773. [symbol] | 430.5 | 4.0 | 592.4 | 480.5 | 34.6 | 19.2 |

Light chain constant
Transcripts per million (TPM)

| Day | IGKC | IGLC1 | IGLC2 | IGLC3 | IGLC7 |
|---|---|---|---|---|---|
| −2 | 500.9 | 138. [symbol] | 21.4 | 47.7 | 0.0 |
| −1 | 134.3 | 62.0 | 8.2 | 24.6 | 2.2 |
| 0 | 73. [symbol] | 6.8 | 0.0 | 6.7 | 0.0 |
| 1 | 31.4 | 2.2 | 0.0 | 2.1 | 0.0 |
| 2 | 53.4 | 5.6 | 3.7 | 7.5 | 0.0 |
| 3 | 112. [symbol] | 21. [symbol] | 3.6 | 5.3 | 0.0 |
| 4 | 200. [symbol] | 27. [symbol] | 2.6 | 13.0 | 0.0 |
| 5 | 53.8 | 2.4 | 2.4 | 4. [symbol] | 0.0 |
| 6 | 156.8 | 14.4 | 5.7 | 19.5 | 0.0 |
| 7 | 390. [symbol] | 136.1 | 16.1 | 32.3 | 0.0 |
| 8 | 628. [symbol] | 183.7 | 35.3 | 50.9 | 2.1 |
| 9 | 17.4 | 2.0 | 0.0 | 0.0 | 0.0 |
| 10 | 954.9 | 150.8 | 26.3 | 60.4 | 0.0 |
| 11 | 2133.4 | 580.3 | 104.1 | 145.4 | 0.0 |
| 12 | 2087.9 | 600.3 | 156.4 | 321.9 | 6.6 |
| 13 | 3053.4 | 671.0 | 123.5 | 362.5 | 4.5 |
| 14 | 1425.3 | 241.7 | 56.1 | 139.2 | 4.2 |
| 15 | 2659.4 | 515.1 | 74.1 | 208.0 | 8.7 |

[symbol]

indicates data missing or illegible when filed

TABLE 15
Levels (TPM) of Ig transcripts in plasma during BM ablation and reconstitution of MM patient 2 in buffy coat - heavy chain variable genes
Heavy chain variable genes
Transcripts per million (TPM)

| | IGHV1- | IGHV1- | IGHV1- | IGHV1- | IGHV1- | IGHV1- | IGHV1- | IGHV1- | IGHV1- | IGHV2- | IGHV2- | IGHV2- | IGHV2- |
|---|---|---|---|---|---|---|---|---|---|---|---|---|---|
| Day | 1 [symbol] | 2 | 24 | 3 | 45 | 46 | 58 | [symbol] | [symbol] -2 | 2 [symbol] | 5 | 70 | 70 |
| −2 | 8.2 | 11.9 | 8.2 | 24.2 | 2.0 | 2.8 | 4.0 | 4.1 | 10.2 | 2.4 | 25.5 | 0.0 | 2.7 |
| −1 | 0.0 | 4. [symbol] | 0.0 | 2.7 | 0.0 | 0.0 | 0.0 | 0.0 | 0.0 | 0.0 | 3.0 | 0.0 | 3.3 |
| 0 | 0.0 | 4. [symbol] | 0.0 | 13.4 | 0.0 | 0.0 | 0.0 | 0.0 | 0.0 | 0.0 | 0.0 | 0.0 | 0.0 |
| 1 | 0.0 | 5.1 | 2.6 | 2.5 | 2. [symbol] | 0.0 | 0.0 | 0.0 | 2.6 | 0.0 | 0.0 | 0.0 | 0.0 |
| 2 | 11.4 | 4.4 | 0.0 | 2.4 | 2.3 | 2.2 | 2.2 | 0.0 | 0.0 | 2.6 | 2.6 | 0.0 | 2. [symbol] |
| 3 | 17.3 | 4.2 | 0.0 | 0.0 | 2.2 | 2.2 | 0.0 | 0.0 | [symbol] | 0.0 | 0.0 | 0.0 | 0.0 |
| 4 | 17.4 | 3.1 | 0.0 | 5.1 | 1.8 | 4.6 | 3.1 | 4.7 | [symbol] .5 | 0.0 | 0.0 | 1.4 | 2.0 |
| 5 | 0.0 | 1.4 | 0.0 | 0.0 | 1.4 | 1.4 | 0.0 | 1.4 | 0.0 | 0.0 | 0.0 | 0.0 | 0.0 |
| 6 | 0.8 | 12.3 | 0.0 | 1.5 | 0.0 | 2.7 | 0.0 | 0.0 | 0.0 | 0.0 | 0.0 | 0.0 | 1.8 |
| 7 | 14.3 | 5.5 | 0.0 | 1.5 | 1.4 | 0.7 | 0.0 | 0.0 | 0.0 | 0.0 | 3.3 | 0.0 | 0.0 |
| 8 | 22.0 | 11.5 | 9.7 | 5.3 | 0.0 | 2.2 | 2.4 | 0.0 | 7.3 | 14.2 | 14.4 | 0.0 | 3.2 |
| 9 | 0.0 | 0.0 | 0.0 | 1 [symbol] .2 | 2.4 | 0.0 | 0.0 | 0.0 | 2.4 | 0.0 | 0.0 | 0.0 | 0.0 |
| 10 | 34.0 | 25. [symbol] | 0.0 | 2.1 | 0.0 | 0. [symbol] | 0.0 | 5.9 | 2.0 | 0.0 | 7.0 | 0.0 | 5.2 |
| 11 | [symbol] | 19.4 | 13.3 | 9.6 | 0.0 | 2 [symbol] .7 | 0.0 | 0.0 | 19.9 | 0.0 | 28.7 | 2. [symbol] | [symbol] |
| 12 | 117.4 | 78.9 | 22.6 | 10.1 | 0.0 | 17.0 | 22. [symbol] | 7.5 | 28.3 | 10.9 | 44.2 | 0.0 | 34.4 |
| 13 | 250. [symbol] | 150.1 | 54.3 | 32.9 | 0.0 | [symbol] | 38.5 | 11.8 | 37.3 | 62.8 | 37.9 | 1.5 | 62.5 |
| 14 | 74.3 | 34.9 | 19.1 | 2.6 | 0.0 | [symbol] | 0.0 | 2.4 | 4. [symbol] | 19.3 | 18.8 | 0.0 | 5.3 |
| 15 | 15 [symbol] .6 | [symbol] | [symbol] | 2.1 | 0.0 | 81.5 | 5.6 | 4.8 | 13.2 | 40.9 | 11.5 | 1.8 | 28.2 |

| | IGHV3- | IGHV3- | IGHV3- | IGHV3- | IGHV3- | IGHV3- | IGHV3- | IGHV3- | IGHV3- | IGHV3- |
|---|---|---|---|---|---|---|---|---|---|---|
| Day | 11 | 13 | 15 | 1 [symbol] | 20 | 21 | 23 | 30 | 33 | 35 |
| −2 | 14.2 | 1.9 | 20.0 | 1. [symbol] | 10.0 | 15.0 | 31.6 | 29.8 | 29.9 | 2. [symbol] |
| −1 | [symbol] | 0.0 | [symbol] .1 | 0.0 | 0.0 | 7.0 | 2.3 | 2.3 | 9.3 | 0.0 |
| 0 | 0.0 | 1. [symbol] | 13.0 | 0.0 | 2.6 | 1.9 | 0.0 | 0.0 | 1.9 | 0.0 |
| 1 | 0.0 | 4. [symbol] | 2.3 | 0.0 | 2.5 | 2.4 | 4.6 | 7.2 | 2.4 | 0.0 |
| 2 | 1.8 | 0.0 | 2.0 | 0.0 | 0.0 | [symbol] | 2.1 | 6.3 | 6.3 | 0.0 |
| 3 | 5.1 | 4.0 | 3.9 | 0.0 | 0.0 | 0.0 | 5.9 | 13.9 | 8.0 | 2. [symbol] |
| 4 | 16.2 | 1.5 | 11.4 | 1.5 | 4.6 | 10.2 | 7.2 | 20.3 | 13.1 | 3.9 |
| 5 | 1.1 | 0.0 | 0.0 | 0.0 | 1.4 | [symbol] | 0.0 | 2.7 | 1.3 | 0.0 |
| 6 | 1.1 | 1.3 | 0.0 | 0.0 | 0.0 | 3.9 | 7.7 | 5.1 | 1 [symbol] .3 | 3.5 |
| 7 | 11.1 | 10.5 | 11.4 | 1.3 | 0.0 | 8.5 | 18.2 | 33.5 | 11.7 | 5.3 |
| 8 | 33.8 | 0.0 | 4.3 | 2.3 | 0.0 | 17.9 | 26.6 | 26.7 | 26.9 | [symbol] .2 |
| 9 | 0.0 | 0.0 | 10.7 | 0.0 | 0.0 | 4.4 | 2.2 | 0.0 | 4.4 | 0.0 |
| 10 | 17.0 | 0.0 | 12.4 | 0.0 | 2.0 | 18.3 | 12.7 | 27.3 | 1 [symbol] .4 | 5. [symbol] |
| 11 | 65.5 | 0.0 | 15.8 | 0.0 | 4.4 | 26.5 | 113.2 | 67.3 | 97.5 | 0.0 |
| 12 | [symbol] | 3.5 | 49.0 | 0.0 | 16.7 | 83.4 | 124.1 | 139.3 | 127.1 | 0.0 |
| 13 | [symbol] | 43.7 | 140.9 | 0.0 | 32.0 | 229.3 | 303.1 | 259.4 | 172.4 | 0.0 |
| 14 | 14.9 | 8.8 | 27.7 | 2.2 | 4.7 | 72.5 | 95.8 | 45.9 | 61.2 | 0.0 |
| 15 | 26.4 | 12.7 | 33.7 | 0.0 | 3.9 | 89.3 | [symbol] | 47.2 | 56.3 | 0.0 |

| Day | IGHV3-38 | IGHV3-43 | IGHV3-48 | IGHV3-49 | IGHV3-53 | IGHV3-64 | IGHV3- [symbol] | IGHV3-7 | IGHV3-72 | IGHV3-73 | IGHV3-74 | IGHV4-28 |
|---|---|---|---|---|---|---|---|---|---|---|---|---|
| −2 | 0.0 | 7.4 | 50.2 | 23.5 | 23.2 | 3.7 | 1. [symbol] | 58.1 | 0.0 | [symbol] | 0.0 | 0.0 |
| −1 | 0.0 | 4.6 | 27.8 | 0.0 | 5.8 | 0.0 | 0.0 | 7.0 | 0.0 | 2.3 | 0.0 | 2.4 |
| 0 | 0.0 | 0.0 | 5.7 | 3.7 | 0.0 | [symbol] | 0.0 | 1.9 | 4.0 | 1. [symbol] | 1.1 | 0.0 |
| 1 | 0.0 | 0.0 | 11.9 | 0.0 | 0.0 | 0.0 | 0.0 | 2.4 | 0.0 | 0.0 | 0.0 | 0.0 |
| 2 | 0.0 | 0.0 | 8.2 | 0.0 | 0.0 | 0.0 | 0.0 | 2.1 | 0.0 | 0.0 | 0.0 | 0.0 |
| 3 | 0.0 | 0.0 | 2.0 | [symbol] | 1.3 | 0.0 | 0.0 | 10.0 | 0.0 | 1.9 | 1.3 | 0.0 |
| 4 | 0.0 | 5.7 | 4.3 | 2.2 | 0.9 | 0.0 | 0.0 | 8.8 | 0.0 | 2.8 | 0.0 | 0.0 |
| 5 | 0.0 | 0.0 | 1.3 | 2.6 | 0.0 | 0.0 | 0.0 | 0.0 | 0.0 | 0.0 | 0.0 | 1.3 |
| 6 | 0.0 | 0.0 | 1.3 | 0.0 | 0.0 | 0.0 | 1. [symbol] | 14.2 | 0.0 | 0.0 | 1.6 | 0.0 |
| 7 | 0.0 | 1.3 | 5.2 | 4.1 | 2.5 | 0.0 | 0.0 | 11.8 | 0.0 | 0.0 | 2.3 | 1.3 |
| 8 | 0.0 | 2.2 | 19.9 | 14.3 | 8.2 | 2.2 | 2.3 | 60.3 | 4.3 | 6.5 | 13.1 | 3.5 |
| 9 | 0.0 | 0.0 | 0.0 | 0.0 | 0.0 | 0.0 | 0.0 | 2.2 | 0.0 | 2.2 | 1.3 | 0.0 |
| 10 | 0.0 | [symbol] .4 | 7.2 | [symbol] | 6.8 | 0.0 | 0.0 | [symbol] | 0.0 | 0.0 | [symbol] .4 | 5.6 |
| 11 | 2.1 | 10.0 | 84.9 | 28.0 | 6.3 | 10.1 | 12.4 | 153.0 | 30.1 | 2.0 | [symbol] .1 | 6.2 |
| 12 | 0.0 | 10.2 | 151.6 | 77.4 | 61.1 | 0.0 | 14.1 | 224.1 | 20.8 | 32.1 | 26.3 | 14.3 |
| 13 | 1.6 | 27.4 | 334. [symbol] | 132.2 | 63.2 | 23.3 | 30.0 | 521.6 | 1 [symbol] .4 | 54.5 | 57. [symbol] | 32. [symbol] |
| 14 | 0.0 | 4.3 | 91.5 | 27.5 | 21.9 | 4.4 | 6.7 | 120.8 | 0.0 | 12.8 | 10.5 | 2.2 |
| 15 | 0.0 | 3.9 | 106. [symbol] | [symbol] | 22.0 | 5.4 | 0.0 | 251.5 | 3.5 | 7.1 | 17.7 | [symbol] |

| Day | IGHV4-31 | IGHV4-34 | IGHV4-39 | IGHV4-4 | IGHV4-59 | IGHV4-61 | IGHV5-51 | IGHV6-1 | IGHV7-81 |
|---|---|---|---|---|---|---|---|---|---|
| −2 | 2. [symbol] | 23.7 | 1.9 | 0.0 | 4.4 | 7.6 | [symbol] | 0.0 | 0.0 |
| −1 | 0.0 | 10.7 | 0.0 | 7.4 | 1.8 | 0.0 | 5.1 | 0.0 | 0.0 |
| 0 | 0.0 | [symbol] | 0.0 | 2.0 | 0.0 | 0.0 | 8.4 | 0.0 | 0.0 |
| 1 | 0.0 | 5.4 | 2.5 | 0.0 | 0.0 | 2.2 | 2.6 | 0.0 | 0.0 |
| 2 | 0.0 | 14.3 | 4.3 | 2.2 | 3.3 | 1.9 | 0.0 | 2.2 | 0.0 |
| 3 | 0.0 | 18.0 | 8.1 | 2.1 | 3.2 | 0.0 | 4.3 | 2.1 | 0.0 |
| 4 | 2.9 | 13.2 | 1.5 | 1.5 | 1.2 | 0.0 | 1. [symbol] | 4.6 | 0.0 |
| 5 | 0.0 | [symbol] | 0.0 | 0.0 | 0.0 | 0.0 | 0.0 | 0.0 | 0.0 |
| 6 | 1.3 | 11.8 | 2.8 | 0.0 | 1.0 | 0.0 | 2.8 | 1.4 | 1.4 |
| 7 | 0.0 | 15.0 | 1.3 | 0.0 | [symbol] | 0.0 | 0.0 | 0.0 | 1.5 |
| 8 | 5.5 | 18.0 | 6.9 | 2.4 | 17.3 | 0.0 | [symbol] 4.7 | 7.2 | 0.0 |
| 9 | 0.0 | 0.0 | 2.3 | 0.0 | 0.0 | 0.0 | 2.4 | 2.4 | 0.0 |
| 10 | 3.5 | 41.9 | 7.5 | 1.0 | 49.9 | 1. [symbol] | 22.6 | 3.9 | 0.0 |
| 11 | 15.1 | 193. [symbol] | 16.7 | 4.3 | 43.1 | [symbol] .7 | 17. [symbol] | 30.6 | 0.0 |
| 12 | 34.7 | 229.9 | 39.1 | 11. [symbol] | 87.5 | 1 [symbol] .2 | 41.7 | 20.4 | 0.0 |
| 13 | [symbol] 8.9 | [symbol] | 94.9 | [symbol] | 129.3 | 23.7 | 93.7 | 4 [symbol] .3 | 0.0 |
| 14 | [symbol] | [symbol] | 20.2 | 13.8 | 29.2 | 2.0 | 45.8 | 4.7 | 0.0 |
| 15 | [symbol] | 74.7 | 32.9 | 15.4 | 31.7 | 12.1 | 39.7 | 31.0 | 0.0 |

[symbol]

indicates data missing or illegible when filed

TABLE 16
Levels (TPM) of Ig transcripts in plasma during BM ablation and reconstitution
of MM patient 2 in buffy coat - Lambda light chain variable genes
Lambda light chain variable genes
Transcripts per million (TPM)

| Day | IGLV10-54 | IGLV11-55 | IGLV1-36 | IGLV1-40 | IGLV1-44 | IGLV1-47 | IGLV1-50 | IGLV1-51 | IGLV2-11 | IGLV2-14 |
|---|---|---|---|---|---|---|---|---|---|---|
| −2 | 2.6 | 0.0 | 0.0 | 4.2 | 11.3 | 10.4 | 0.0 | 4.3 | 2.1 | 19.2 |
| −1 | 0.0 | 0.0 | 0.0 | 7.8 | 8.0 | 0.0 | 0.0 | 2.7 | 0.0 | 5.3 |
| 0 | 0.0 | 0.0 | 0.0 | 0.0 | 1.7 | 0.0 | 0.0 | 0.0 | 0.0 | 0.0 |
| 1 | 0.0 | 0.0 | 0.0 | 0.0 | 2.1 | 0.0 | 0.0 | 0.0 | 0.0 | 0.0 |
| 2 | 0.0 | 0.0 | 0.0 | 0.0 | 1.8 | 0.0 | 0.0 | 0.0 | 0.0 | 0.0 |
| 3 | 0.0 | 0.0 | 0.0 | 6.6 | 3.5 | 0.0 | 2.2 | 6.8 | 0.0 | 4.5 |
| 4 | 0.0 | 0.0 | 0.0 | 1.6 | 6.4 | 0.0 | 0.0 | 3.3 | 3.3 | 4.9 |
| 5 | 0.0 | 0.0 | 0.0 | 2.9 | 0.0 | 0.0 | 0.0 | 0.0 | 0.0 | 0.0 |
| 6 | 1.7 | 0.0 | 0.0 | 2.8 | 0.0 | 1.4 | 2.9 | 1.5 | 0.0 | 2.9 |
| 7 | 1.8 | 0.0 | 0.0 | 11.6 | 0.0 | 2.9 | 0.0 | 3.0 | 3.0 | 4.5 |
| 8 | 6.2 | 0.0 | 2.7 | 12.4 | 7.7 | 17.4 | 0.0 | 5.1 | 5.1 | 12.0 |
| 9 | 0.0 | 0.0 | 0.0 | 0.0 | 1.9 | 0.0 | 0.0 | 0.0 | 0.0 | 0.0 |
| 10 | 2.5 | 0.0 | 0.0 | 6.1 | 6.3 | 18.2 | 0.0 | 0.0 | 12.6 | 22.8 |
| 11 | 5.6 | 0.0 | 0.0 | 121.9 | 21.1 | 40.6 | 0.0 | 23.3 | 39.7 | 62.1 |
| 12 | 21.3 | 0.0 | 0.0 | 84.4 | 70.7 | 57.6 | 0.0 | 35.5 | 51.5 | 115.2 |
| 13 | 23.6 | 0.0 | 24.0 | 171.01 | 115.9 | 114.0 | 0.0 | 74.9 | 70.0 | 239.6 |
| 14 | 12.0 | 0.0 | 2.6 | 36.5 | 41.6 | 26.7 | 0.0 | 60.2 | 20.1 | 22.4 |
| 15 | 12.3 | 0.0 | 0.0 | 78.4 | 55.4 | 52.2 | 0.0 | 22.8 | 22.8 | 53.0 |

| Day | IGLV2-1 [symbol] | IGLV2-23 | IGLV2-33 | IGLV2-8 | IGLV3-1 | IGLV3-10 | IGLV3-12 | IGLV3-1 [symbol] | IGLV3-19 |
|---|---|---|---|---|---|---|---|---|---|
| −2 | 0.0 | 10.0 | 0.0 | 2.7 | 10.9 | 0.0 | 2.7 | 0.0 | 9.7 |
| −1 | 0.0 | 5.4 | 0.0 | 1.7 | 2.7 | 0.0 | 0.0 | 0.0 | 9.0 |
| 0 | 0.0 | 0.0 | 0.0 | 0.0 | 2.2 | 0.0 | 0.0 | 0.0 | 0.0 |
| 1 | 0.0 | 0.0 | 0.0 | 0.0 | 0.0 | 2.9 | 0.0 | 0.0 | 0.0 |
| 2 | 0.0 | 0.0 | 0.0 | 0.0 | 2.4 | 0.0 | 0.0 | 0.0 | 2.7 |
| 3 | 0.0 | 3.1 | 0.0 | 1.5 | 2.3 | 0.0 | 0.0 | 0.0 | 5.0 |
| 4 | 0.0 | 0.0 | 0.0 | 1.1 | 3.3 | 0.0 | 0.0 | 0.0 | 5.5 |
| 5 | 0.0 | 0.0 | 0.0 | 0.0 | 0.0 | 0.0 | 1.8 | 0.0 | 0.0 |
| 6 | 0.0 | 0.0 | 0.0 | 0.0 | 16.3 | 0.0 | 0.0 | 0.0 | 0.0 |
| 7 | 0.0 | 6.0 | 0.0 | 0.9 | 16.6 | 1.6 | 0.0 | 0.0 | 5.0 |
| 8 | 0.0 | 3.9 | 0.0 | 0.0 | 38.9 | 2.6 | 3.2 | 0.0 | 11.6 |
| 9 | 0.0 | 0.0 | 0.0 | 0.0 | 0.0 | 0.0 | 0.0 | 0.0 | 0.0 |
| 10 | 0.0 | 0.0 | 0.0 | 3.9 | 23.3 | 0.0 | 0.0 | 0.0 | 0.0 |
| 11 | 0.0 | 6.4 | 0.0 | 5.9 | 35.3 | 2.5 | 5.8 | 0.0 | 36.7 |
| 12 | 20.3 | 17.8 | 0.0 | 27.7 | 74.0 | 4.2 | 0.0 | 0.0 | 91.0 |
| 13 | 6.1 | 34.8 | 0.0 | 30.4 | 137.0 | 24.9 | 2.2 | 0.0 | 212.5 |
| 14 | 2.9 | 11.8 | 0.0 | 12.7 | 33.0 | 10.8 | 0.0 | 0.0 | 56.4 |
| 15 | 0.0 | 14.4 | 0.0 | 22.6 | 71.2 | 8.9 | 5.1 | 0.0 | 104.3 |

| Day | IGLV3-21 | IGLV3-22 | IGLV3-25 | IGLV3-27 | IGLV3-32 | IGLV3-9 | IGLV4-3 | IGLV4-60 | IGLV4-69 | IGLV5-37 |
|---|---|---|---|---|---|---|---|---|---|---|
| −2 | 56.6 | 0.0 | 0.0 | 0.0 | 0.0 | 1.6 | 0.0 | 0.0 | 2.0 | 2.5 |
| −1 | 10.7 | 0.0 | 0.0 | 3.0 | 0.0 | 0.0 | 0.0 | 0.0 | 0.0 | 0.0 |
| 0 | 3.3 | 0.0 | 0.0 | 0.0 | 0.0 | 0.0 | 0.0 | 0.0 | 0.0 | 0.0 |
| 1 | 0.0 | 0.0 | 0.0 | 0.0 | 0.0 | 0.0 | 0.0 | 0.0 | 0.0 | 0.0 |
| 2 | 0.0 | 0.0 | 0.0 | 0.0 | 0.0 | 0.0 | 0.0 | 0.0 | 0.0 | 0.0 |
| 3 | 0.0 | 0.0 | 0.0 | 0.0 | 0.0 | 0.0 | 0.0 | 0.0 | 0.0 | 0.0 |
| 4 | 1.7 | 0.0 | 0.0 | 0.0 | 0.0 | 0.0 | 0.0 | 0.0 | 0.0 | 0.0 |
| 5 | 0.0 | 0.0 | 0.0 | 0.0 | 0.0 | 0.0 | 0.0 | 0.0 | 0.0 | 0.0 |
| 6 | 3.0 | 0.0 | 0.0 | 0.0 | 0.0 | 1.1 | 0.0 | 0.0 | 0.0 | 0.0 |
| 7 | 4.5 | 0.0 | 0.0 | 0.0 | 0.0 | 1.2 | 0.0 | 0.0 | 1.4 | 0.0 |
| 8 | 6.3 | 0.0 | 0.0 | 0.0 | 0.0 | 3.9 | 0.0 | 0.0 | 0.0 | 0.0 |
| 9 | 0.0 | 0.0 | 0.0 | 0.0 | 0.0 | 0.0 | 0.0 | 0.0 | 0.0 | 0.0 |
| 10 | 12.5 | 0.0 | 0.0 | 0.0 | 0.0 | 1.6 | 0.0 | 2.6 | 0.0 | 0.0 |
| 11 | 30.5 | 0.0 | 0.0 | 0.0 | 0.0 | 1.8 | 2.3 | 0.0 | 17.1 | 0.0 |
| 12 | 56.5 | 0.0 | 4.4 | 0.0 | 0.0 | 16.4 | 0.0 | 4.8 | 5.5 | 6.8 |
| 13 | 111.3 | 0.0 | 19.7 | 2.0 | 0.0 | 34.5 | 0.0 | 15.3 | 76.5 | 4.1 |
| 14 | 30.3 | 0.0 | 5.5 | 0.0 | 0.0 | 5.8 | 0.0 | 0.0 | 20.7 | 0.0 |
| 15 | 39.6 | 0.0 | 2.3 | 0.0 | 0.0 | 4.9 | 0.0 | 7.5 | 91.6 | 0.0 |

| Day | IGLV5-45 | IGLV5-48 | IGLV [symbol] -52 | IGLV6-57 | IGLV7-43 | IGLV7-46 | IGLV8-61 | IGLV9-49 |
|---|---|---|---|---|---|---|---|---|
| −2 | 0.0 | 0.0 | 0.0 | 2.0 | 2.3 | 4.6 | 2.0 | 0.0 |
| −1 | 0.0 | 0.0 | 0.0 | 2.5 | 0.0 | 0.0 | 0.0 | 0.0 |
| 0 | 0.0 | 0.0 | 0.0 | 0.0 | 0.0 | 0.0 | 0.0 | 0.0 |
| 1 | 0.0 | 0.0 | 3.0 | 0.0 | 0.0 | 0.0 | 0.0 | 0.0 |
| 2 | 0.0 | 0.0 | 2.6 | 0.0 | 0.0 | 0.0 | 0.0 | 0.0 |
| 3 | 2.3 | 0.0 | 0.0 | 2.3 | 0.0 | 0.0 | 0.0 | 0.0 |
| 4 | 0.0 | 0.0 | 0.0 | 0.8 | 1.8 | 0.0 | 0.0 | 0.0 |
| 5 | 1.5 | 0.0 | 0.0 | 0.0 | 0.0 | 0.0 | 0.0 | 0.0 |
| 6 | 0.0 | 0.0 | 1.5 | 0.0 | 0.0 | 0.0 | 0.0 | 0.0 |
| 7 | 0.0 | 0.0 | 2.7 | 0.7 | 0.0 | 4.8 | 5.6 | 0.0 |
| 8 | 0.0 | 0.0 | 0.0 | 2.4 | 5.6 | 0.0 | 2.4 | 0.0 |
| 9 | 0.0 | 0.0 | 0.0 | 0.0 | 0.0 | 0.0 | 0.0 | 0.0 |
| 10 | 0.0 | 0.0 | 0.0 | 3.0 | 2.3 | 0.0 | 5.9 | 0.0 |
| 11 | 0.0 | 0.0 | 6.7 | 1.1 | 2.5 | 5.0 | 24.0 | 0.0 |
| 12 | 0.0 | 2.3 | 5.5 | 4.8 | 14.9 | 17.1 | 20.5 | 3.8 |
| 13 | 9.1 | 2.1 | 0.0 | 13.6 | 26.9 | 17.3 | 51.8 | 10.3 |
| 14 | 0.0 | 0.0 | 0.0 | 6.0 | 8.1 | 2.7 | 11.8 | 0.0 |
| 15 | 4.2 | 0.0 | 0.0 | 6.1 | 4.5 | 6.7 | 5.8 | [symbol] .0 |

[symbol]

indicates data missing or illegible when filed

TABLE 17
Levels (TPM) of Ig transcripts in plasma during BM ablation and reconstitution of MM patient 2 in buffy coat - Kappa light chain variable genes
Kappa light chain variable genes
Transcript per million (TPM)

| | IGKV1- | IGKV1- | IGKV1- | IGKV1- | IGKV1- | IGKV1- | IGKV1- | IGKV1- | IGKV1- | IGKV1- | IGKV1- | IGKV1D- | IGKV1D- | IGKV1D- |
|---|---|---|---|---|---|---|---|---|---|---|---|---|---|---|
| Day | 12 | 16 | 17 | 27 | 33 | 37 | [symbol] 9 | 5 | 6 | 8 | 9 | 12 | 13 | 16 |
| −2 | 38.6 | [symbol] | 14.4 | 0.0 | 0.0 | 0.0 | 0.0 | 14.3 | 7.1 | 7. [symbol] | 2.1 | 0.0 | 2.4 | 17.6 |
| −1 | 7.3 | 14.9 | 0.0 | 2.9 | 0.0 | 0.0 | 0.0 | 6. [symbol] | 0.0 | 2.4 | 0.0 | 0.0 | 0.0 | 0.0 |
| 0 | 2. [symbol] | 0.0 | 2.4 | 0.0 | 0.0 | 0.0 | 0.0 | 4.0 | 0.0 | 2.2 | 0.0 | 0.0 | 0.0 | 0.0 |
| 1 | 3.6 | 0.0 | 0.0 | 0.0 | 0.0 | 0.0 | 0.0 | 0.0 | 0.0 | 2.7 | 0.0 | 0.0 | 0.0 | 3. [symbol] |
| 2 | 5.4 | 2.6 | 0.0 | 0.0 | 0.0 | 0.0 | 0.0 | 4.5 | 2. [symbol] | [symbol] | 10.1 | 0.0 | 0.0 | 2.6 |
| 3 | 3.9 | 0.0 | 0.0 | 0.0 | 0.0 | 0.0 | 0.0 | 5.8 | 0.0 | [symbol] | 0.0 | 0.0 | 0.0 | 0.0 |
| 4 | 10.6 | 0.0 | 5.5 | 5.4 | 0.0 | 0.0 | 0.0 | 5.2 | 0.0 | [symbol] | 3.2 | 0.0 | 0.0 | 0.0 |
| 5 | 11.4 | 0.0 | 0.0 | 0.0 | 0.0 | 0.0 | 0.0 | 4.8 | 1. [symbol] | 1.0 | 0.0 | 0.0 | 0.0 | 3.3 |
| 6 | 7.8 | 0.0 | 1. [symbol] | 0.0 | 0.0 | 0.0 | 0.0 | 0.0 | 0.0 | 1.4 | 0.0 | 0.0 | 0.0 | 0.0 |
| 7 | 13.6 | 30.1 | 13.4 | 4.9 | 0.0 | 0.0 | 0.0 | 15.5 | 1. [symbol] | 3.5 | 4.3 | 0.0 | 0.0 | 3.3 |
| 8 | [symbol] | 14.4 | 17.3 | 0.0 | 0.0 | 0.0 | 0.0 | 24. [symbol] | 2.9 | 15.4 | 3.1 | 0.0 | 0.0 | 0.0 |
| 9 | 1.4 | 0.0 | 0.0 | 2.8 | 0.0 | 0.0 | 0.0 | 0.0 | 0.0 | 0.0 | 0.0 | 0.0 | 0.0 | 2.8 |
| 10 | 35.8 | 30.4 | 11.7 | 4.6 | 0.0 | 0.0 | 0.0 | 22.6 | 0.0 | 7.0 | 0.0 | 0.0 | 0.0 | 49.3 |
| 11 | [symbol] | 28.6 | 26.1 | 48. [symbol] | 0.0 | 0.0 | 0.0 | 22.7 | 5.1 | [symbol] | 9.7 | 0.0 | 0.0 | [symbol] |
| 12 | 117.6 | 105.6 | 32.7 | 23.6 | 0.0 | 0.0 | 0.0 | 119.3 | 30. [symbol] | 44.6 | 2.0 | 0.0 | 1.9 | [symbol] |
| 13 | 243.6 | 161.6 | 99.0 | 60.6 | 2.0 | 0.0 | 0.0 | 328.4 | [symbol] | [symbol] 3.3 | [symbol] | 0.0 | 7.3 | 41.8 |
| 14 | 504.3 | 16.6 | 16.8 | 0.0 | 0.0 | 0.0 | 0.0 | 58.1 | 11.1 | 34.4 | 24.1 | 0.0 | 0.0 | [symbol] |
| 15 | 195.4 | 25.4 | 32.3 | 15.6 | 3.2 | 0.0 | 0.0 | 101.6 | 27.3 | 32.6 | [symbol] | 0.0 | 4.4 | [symbol] |

| | IGKV1D- | IGKV1D- | IGKV1D- | IGKV1D- | IGKV1D- | IGKV1D- | IGKV1D- | IGKV1OR2- | IGKV2- | IGKV2- | IGKV2- |
|---|---|---|---|---|---|---|---|---|---|---|---|
| Day | 17 | 33 | 37 | 39 | 42 | 43 | 8 | 10 [symbol] | 24 | 28 | 30 |
| −2 | 2.7 | 0.0 | 0.0 | 0.0 | 0.0 | 0.0 | 2.6 | 0.0 | 22.6 | 0.0 | 11. [symbol] |
| −1 | 0.0 | 0.0 | 0.0 | 0.0 | 0.0 | 0.0 | 0.0 | 0.0 | 8.4 | 0.0 | 0.0 |
| 0 | 1.3 | 0.0 | 0.0 | 0.0 | 0.0 | 0.0 | 0.0 | 0.0 | 18.4 | 0.0 | 0.0 |
| 1 | 0.0 | 0.0 | 0.0 | 0.0 | 0.0 | 0.0 | 0.0 | 0.0 | [symbol] | 0.0 | 0.0 |
| 2 | 0.0 | 0.0 | 0.0 | 0.0 | 0.0 | 0.0 | 0.0 | 0.0 | 0.0 | 0.0 | 0.0 |
| 3 | 0.0 | 0.0 | 0.0 | 0.0 | 0.0 | 0.0 | 0.0 | 0.0 | 0.0 | 0.0 | 0.0 |
| 4 | 0.0 | 0.0 | 0.0 | 0.0 | 0.0 | 0.0 | 2.1 | 0.0 | 0.0 | 0.0 | 1.7 |
| 5 | 0.0 | 0.0 | 0.0 | 0.0 | 0.0 | 0.0 | 0.0 | 0.0 | 0.0 | 0.0 | 1. [symbol] |
| 6 | 0.0 | 0.0 | 0.0 | 0.0 | 0.0 | 0.0 | 0.0 | 0.0 | 0.0 | 0.0 | 0.0 |
| 7 | 0.0 | 0.0 | 0.0 | 1.7 | 0.0 | 0.0 | [symbol] | 0.0 | 1.6 | 0.0 | 1. [symbol] |
| 8 | 0.0 | 0.0 | 0.0 | 0.0 | 0.0 | 0.0 | 1.5 | 0.0 | 0.0 | 0.0 | 0.0 |
| 9 | 0.0 | 0.0 | 0.0 | 0.0 | 0.0 | 0.0 | 1.5 | 0.0 | 29.3 | 0.0 | 0.0 |
| 10 | 0.0 | 0.0 | 0.0 | 0.0 | 0.0 | 0.0 | [symbol] | 0.0 | [symbol] | 0.0 | 0.0 |
| 11 | 1.4 | 0.0 | 0.0 | 0.0 | 0.0 | 0.0 | 1.4 | 0.0 | [symbol] | 0.0 | [symbol] |
| 12 | 5.3 | 0.0 | 0.0 | 2.2 | 0.0 | 1.2 | 0.0 | 0.0 | 20.8 | 0.0 | 27.1 |
| 13 | 9.7 | 0.0 | 0.0 | 12.8 | 0.0 | 2.2 | 6.6 | 0.0 | 20.8 | 5.6 | [symbol] |
| 14 | 0.0 | 0. [symbol] | 0.0 | 3.5 | 0.0 | 0.5 | 3.0 | 0.0 | 13.2 | 5.3 | 7.9 |
| 15 | 0.0 | 3.2 | 0.0 | 6.9 | 0.0 | 0.0 | 0.0 | 0.0 | 4.4 | [symbol] .0 | [symbol] |

| | IGKV2- | IGKV2D- | IGKV2D- | IGKV2D- | IGKV2D- | IGKV2D- | IGKV2D- | IGKV3- | IGKV3- | IGKV3- | IGKV3- |
|---|---|---|---|---|---|---|---|---|---|---|---|
| Day | 40 | 24 | 26 | 28 | 29 | 30 | 40 | 11 | 15 | 20 | 7 |
| −2 | 0.0 | 0.0 | 0.0 | 0.0 | 4. [symbol] | 0.0 | 0.0 | 17. [symbol] | 1. [symbol] | [symbol] | 0.0 |
| −1 | 0.0 | 0.0 | 0.0 | 0.0 | 2. [symbol] | 0.0 | 0.0 | 0.0 | 0.0 | 10.7 | 0.0 |
| 0 | 0.0 | 0.0 | 0.0 | 0.0 | 0.0 | 0.0 | 0.0 | 2.3 | 0.0 | 0.0 | 0.0 |
| 1 | 0.0 | 0.0 | 0.0 | 0.0 | 0.0 | 0.0 | 0.0 | 0.0 | 2.3 | 0.0 | 0.0 |
| 2 | 0.0 | 0.0 | 0.0 | 0.0 | 0.0 | 0.0 | 0.0 | 0.0 | 0.0 | 4. [symbol] | 0.0 |
| 3 | 0.0 | 0.0 | 0.0 | 0.0 | 0.0 | 0.0 | 0.0 | 0.0 | 1. [symbol] | [symbol] | 0.0 |
| 4 | 0.0 | 0.0 | 0.0 | 0.0 | 0.0 | 0.0 | 0.0 | 1.7 | 0.0 | [symbol] | 0.0 |
| 5 | 0.0 | 0.0 | 0.0 | 0.0 | 0.0 | 0.0 | 0.0 | 0.0 | 0.0 | 0.0 | 0.0 |
| 6 | 0.0 | 0.0 | 0.0 | 0.0 | 0.0 | 0.0 | 0.0 | 1.5 | 2.5 | 4.4 | 2.4 |
| 7 | 0.0 | 0.0 | 0.0 | 0.0 | 2. [symbol] | 0.0 | 0.0 | 3.1 | 1. [symbol] | 21.0 | 0.0 |
| 8 | 0.0 | 0.0 | 0.0 | 0.0 | 0.0 | 0.0 | 0.0 | [symbol] .4 | 2.1 | 23.1 | 0.0 |
| 9 | 0.0 | 0.0 | 0.0 | 0.0 | 0.0 | 0.0 | 0.0 | [symbol] | 0.0 | 0.0 | 0.0 |
| 10 | 0.0 | 0.0 | 0.0 | 0.0 | 4.4 | [symbol] .4 | 0.0 | [symbol] | 7.0 | 54.5 | 0.0 |
| 11 | 0.0 | 0.0 | 0.0 | 0.0 | [symbol] | 0.0 | 0.0 | 33.9 | [symbol] | 112.0 | 0.0 |
| 12 | 0.0 | 0.0 | 0.0 | 0.0 | [symbol] .7 | 4.0 | 0.0 | 39.1 | 62. [symbol] | 136.8 | 0.0 |
| 13 | 0.0 | 0.0 | 0.0 | 0.0 | [symbol] | 0.0 | 2. [symbol] | 113.2 | 233. [symbol] | 241.1 | 0.0 |
| 14 | 0.0 | 0.0 | 0.0 | 0.0 | 0.0 | 0.0 | 0.0 | [symbol] | 125.4 | 72.9 | 0.0 |
| 15 | 0.0 | 2.2 | 2.2 | 0.0 | 2.2 | 0.0 | 0.0 | 33. [symbol] | 23 [symbol] .2 | 97. [symbol] | 0.0 |

| | IGKV3D- | IGKV3D- | IGKV3D- | IGKV3D- | IGKV3OR2- | IGKV4- | IGKV5- | IGKV6- | IGKV [symbol] D- | IGKV [symbol] D- |
|---|---|---|---|---|---|---|---|---|---|---|
| Day | 11 | 15 | 20 | 7 | 26 [symbol] | 1 | 2 | 21 | 21 | 41 |
| −2 | 0.0 | 0.0 | 0.0 | 0.0 | 0.0 | 26.7 | 0.0 | [symbol] | 0.0 | 0.0 |
| −1 | 0.0 | 0.0 | 0.0 | 0.0 | 0.0 | 1. [symbol] | 0.0 | [symbol] | 0.0 | 0.0 |
| 0 | 0.0 | 0.0 | 0.0 | 0.0 | 0.0 | 2.6 | 0.0 | 0.0 | 0.0 | 0.0 |
| 1 | 0.0 | 0.0 | 0.0 | 0.0 | 0.0 | 5.1 | 0.0 | 0.0 | 0.0 | 0.0 |
| 2 | 0.0 | 0.0 | 0.0 | 0.0 | 0.0 | 5.9 | 0.0 | 2.3 | 0.0 | 0.0 |
| 3 | 0.0 | 0.0 | 0.0 | 0.0 | 0.0 | 2. [symbol] | 0.0 | 0.0 | 0.0 | 0.0 |
| 4 | 0.0 | 0.0 | 0.0 | 0.0 | 0.0 | 6.1 | 0.0 | 0.0 | 0.0 | 0.0 |
| 5 | 0.0 | 0.0 | 0.0 | 0.0 | 0.0 | 0.0 | 0.0 | 0.0 | 0.0 | 0.0 |
| 6 | 0.0 | 0.0 | 1.2 | 0.0 | 0.0 | 0. [symbol] | 0.0 | 0.0 | 0.0 | 1.7 |
| 7 | 3.1 | 0.0 | 0.0 | 0.0 | 0.0 | 17.0 | 1.4 | 1.5 | 0.0 | 0.0 |
| 8 | 0.0 | 0.0 | 0.0 | 0.0 | 0.0 | 30.2 | 2. [symbol] | 0.0 | 0.0 | 0.0 |
| 9 | 0.0 | 0.0 | 0.0 | 0.0 | 0.0 | 1.5 | 0.0 | 0.0 | 0.0 | 0.0 |
| 10 | 0.0 | 0.0 | 0.0 | 0.0 | 0.0 | 9.9 | 0.0 | 0.0 | 0.0 | 0.0 |
| 11 | 0.0 | 0.0 | 1. [symbol] | 0.0 | 0.0 | 135.2 | 11.2 | 0.0 | [symbol] .7 | 0.0 |
| 12 | 3. [symbol] | 0.0 | [symbol] .2 | 0.0 | 0.0 | 51.3 | 1.9 | 7.7 | 7. [symbol] | 0.0 |
| 13 | 0.0 | 1. [symbol] | 4.3 | 0.0 | 0.0 | 191.0 | [symbol] | 10.4 | 3.4 | 0.0 |
| 14 | 0.0 | 0.0 | 4.1 | 0.0 | 0.0 | 42.1 | 0.0 | 0.0 | 0.0 | 0.0 |
| 15 | 2.2 | 2.2 | 1. [symbol] | 0.0 | 0.0 | [symbol] 0.7 | 4. [symbol] | 0.0 | 0.0 | 0.0 |

[symbol]

indicates data missing or illegible when filed

To test whether cf-mRNA profiling can be used to monitor the levels of the malignant Ig clone, the cf-mRNA from plasma of these patients was sequenced every day for two weeks after chemotherapy and transplant. While Patient 1 showed no apparent reduction of the malignant clone after therapy (FIG. 7D), Patient 2 showed decreased levels of the predominant Ig variants in cf-mRNA after Melphalan-induced apoptosis of plasma cells (FIGS. 2B-D and FIGS. 7A-7C). By day 10, the immune profile was no longer dominated by clonal Ig combinations, indicating successful therapy and BM reconstitution (FIGS. 2B-D). In contrast, RNA-Seq performed on the matching buffy coat fraction throughout the study showed very limited information regarding the malignant Ig transcripts (FIG. 2C and FIGS. 7A-7E), supporting the potential of cf-mRNA to non-invasively capture BM activity.

### Example 11

### cf-mRNA Captures Hematopoietic Lineage Transcriptional Activity During BM Ablation and Reconstitution

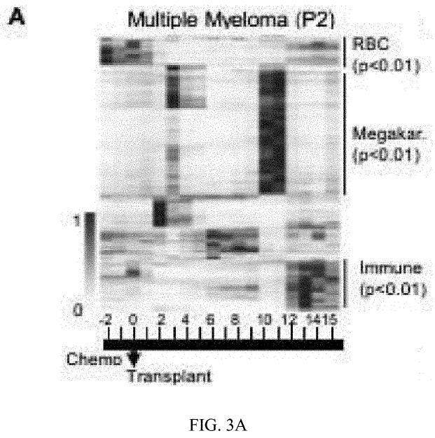

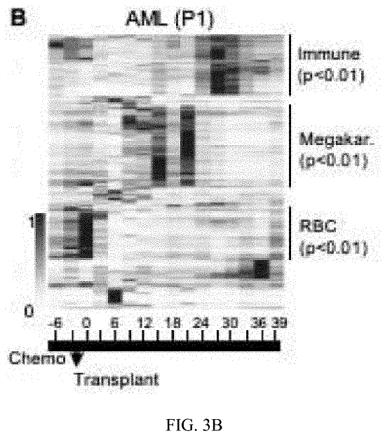

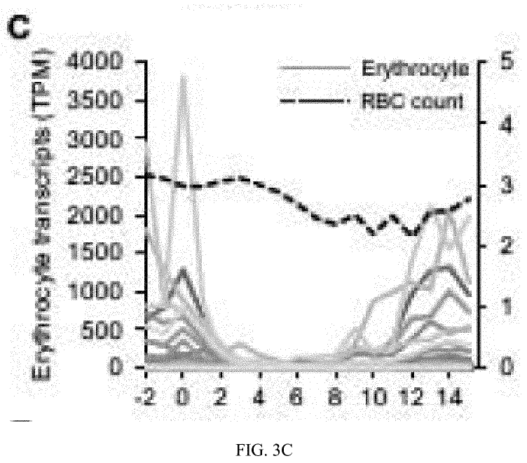

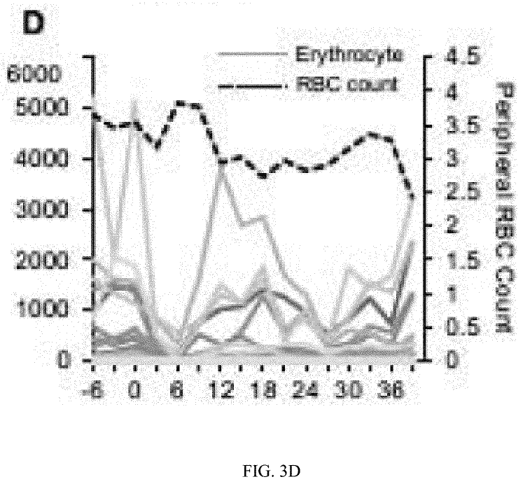

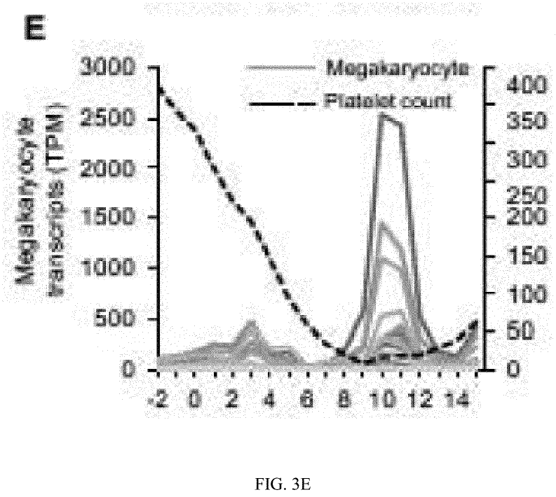

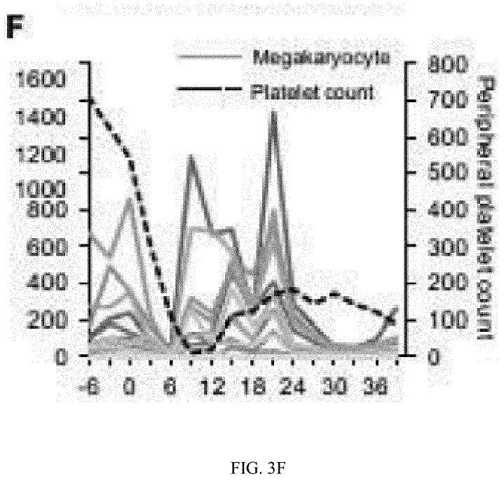

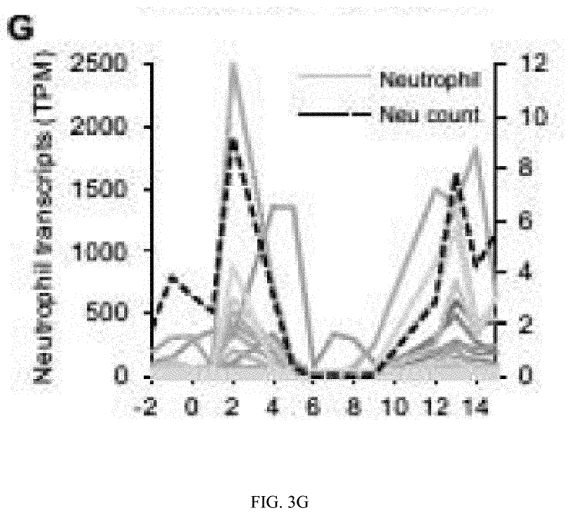

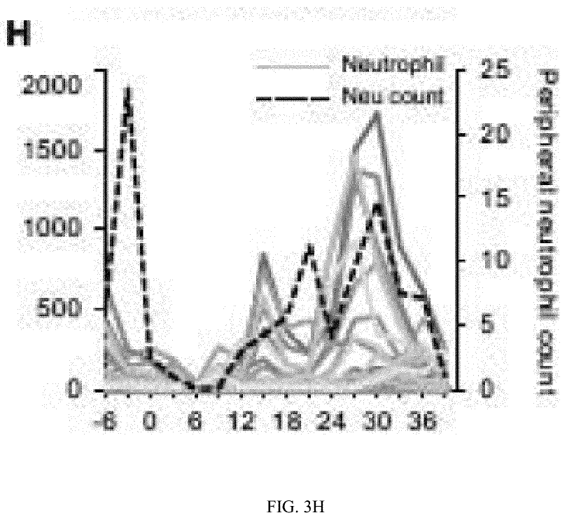

To gain further insights into the ability of circulating mRNA to reveal BM transcriptional activity, the BM ablation and reconstitution dynamics were followed after autologous transplants in cf-mRNA, using the prototypical MM Patient 2. Additionally, acute myeloid leukemia (AML) patients were investigated who underwent submyeloablative treatment followed by allogeneic transplant (see examples, AML Patients 1 and 2 were monitored for 8 weeks, Patient 3 was discharged 2 weeks after transplant). Unsupervised clustering of transcripts detected in plasma cf-mRNA of MM and AML patients identified temporal patterns of expression for several groups of genes (FIGS. 3A, B). Both Gene Ontology enrichment analysis and RNA-seq data from Blueprint Consortium indicated that many of the identified components correspond to specific hematopoietic lineages (FIGS. 3A, B). Therefore, the dynamics of hematopoietic lineage-specific transcripts as listed in Table 9 (i.e., erythrocytes, megakaryocytes, and neutrophils) were examined in detail in circulation during BM ablation and reconstitution.

TABLE 9
List of indicated hematopoietic lineage-specific transcripts

| Erythrocyte | Megakaryocyte | T-cells | T-cells | T-cells | Neutrophil | Immature neutrophil | Mature neutrophil |
|---|---|---|---|---|---|---|---|
| SLC4A1 | ITGA2B | PDZD4 | TRGV10 | TRAV23DV6 | PGLYRP1 | ELANE | S100A12 |
| TF | RAB27B | TBX21 | TRGV4 | TRAV25-1 | LTF | PRTN3 | KRT23 |
| AVP | GUCY1 [symbol] 3 | CHRNA3 | TRBV6-1 | TRAV41 | ATP2C2 | AZU1 | FCGR3B |
| RUNDC3A | GP6 | [symbol] IRPG | TRBV9 | [symbol] BH-AS1 | VNN3 | CT [symbol] G | PI3 |
| SOX6 | HGD | PITPNM2 | TRBV6-5 | AC011893.3 | CRISP3 | RNASE | STEAP4 |
| TSPO2 | PF4 | GZMH | TRBV5-6 | RP11-73O6.3 | CTSG | PGLYRP1 | PROk2 |
| H [symbol] Z | CLEC1B | GZMB | TRBV4-2 | TRBV10-2 | OLFM4 | MMP8 | CXCR1 |
| TMCC2 | CMTM5 | GZMK | TRBV20-1 | TRBV5-4 | KRT23 | | CXCR2 |
| SELENBP1 | GP9 | GNLY | TRBC1 | RP11-144L [symbol] 4 | MMP8 | | CD177 |
| ALAS2 | SELP | CD2 | TRBV27 | LINC009 [symbol] 7 | ARG1 | | KCNJ15 |
| EP [symbol] 42 | DNM3 | CD160 | TRAV2 | TRBV30 | EPX | | ALPL |
| GYPA | LY6G6F | ELOVL4 | TRAV3 | TRBV3-1 | PI3 | | |
| C17orf99 | LY6G6D | EPHX2 | TRAV4 | TRBV11-2 | CRISP2 | | |
| HBA2 | XXbac-BPG32J3.19 | SARDH | TRAV10 | A2M-AS1 | STEAP4 | | |
| RHCE | RP11-879F14.2 | KLRC1 | TRAV12-2 | LINC01550 | LCN2 | | |
| HBG2 | | FGFBP2 | TRAV13-2 | RP11-291B21.2 | PRG3 | | |
| TRIM10 | | ARL5C | TRAV14DV4 | TRAV1-2 | KCNJ15 | | |
| HBA1 | | RORC | TRAV12-3 | RP11-204N11.1 | ALPL | | |
| HBM | | GZMA | TRAV17 | RP11-158G18.1 | FCGR3B | | |
| H [symbol] G1 | | SCML4 | TRAV19 | RP11-415F23.3 | S100A12 | | |
| UCA1 | | EPHA1 | TRAV20 | TRBV15 | PROK2 | | |
| GYPB | | KLRF1 | TRAV21 | TRBV12-4 | CXCR1 | | |
| CTD-3154N5.2 | | PPP1R1C | DTHD1 | CXCR5 | CAMP | | |
| AC104389.1 | | CD8A | KLRC2 | THEMIS | RNASE3 | | |
| | | PPP2R2B | RP11-415F23.4 | LRRN3 | CEACAM3 | | |
| | | TRAT1 | RP11-104L21.3 | CCR9 | AZU1 | | |
| | | CTLA4 | TRBV12-3 | PRF1 | A [symbol] CA13 | | |
| | | MAL | TRBV10-3 | FCRL6 | CXCR2 | | |
| | | CD8B | TRBV13 | TIGIT | CTD-3088G3.8 | | |
| | | | | ADARB2 | PRTN3 | | |
| | | | | | ELANE | | |
| | | | | | CD177 | | |
| | | | | | LINC00671 | | |
| | | | | | ORM2 | | |
| | | | | | ORM1 | | |
| | | | | | HP | | |
| | | | | | RP11-678G14.4 | | |

[symbol]

indicates data missing or illegible when filed

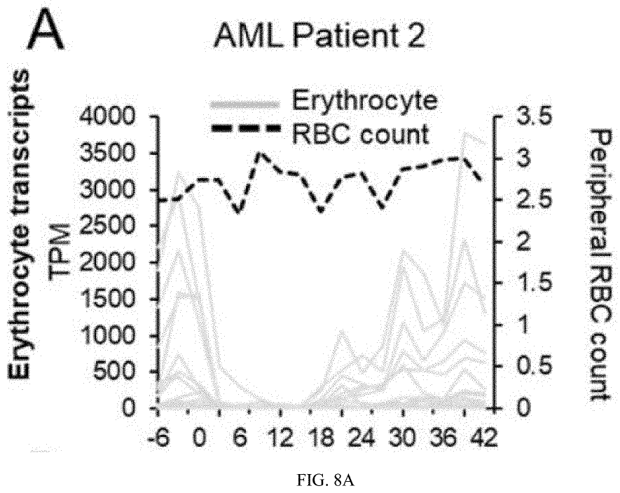

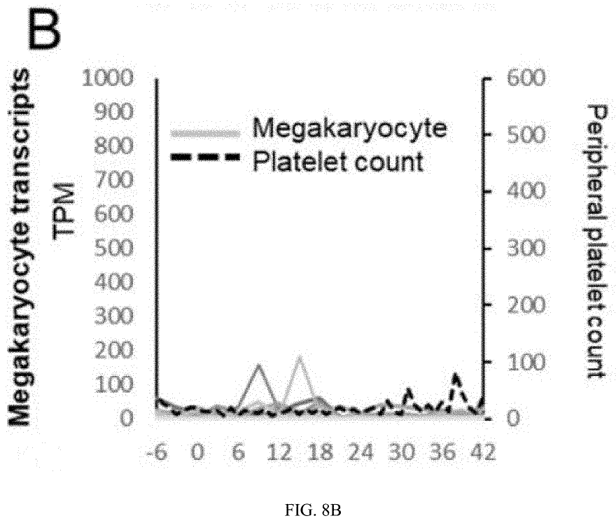

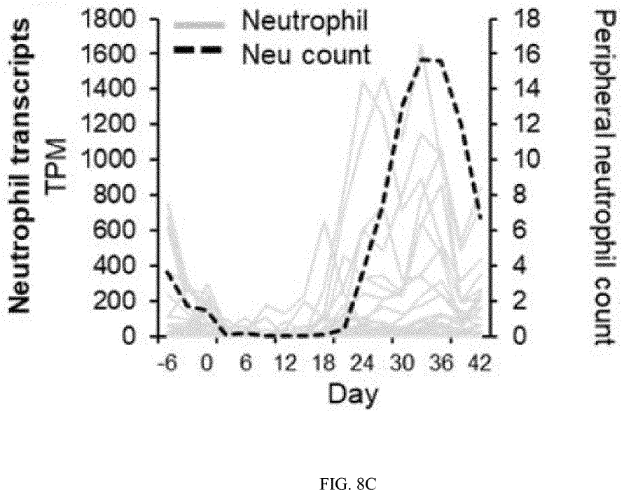

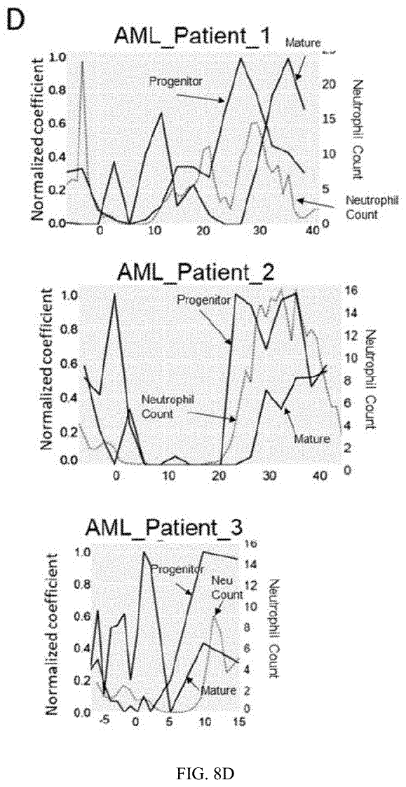

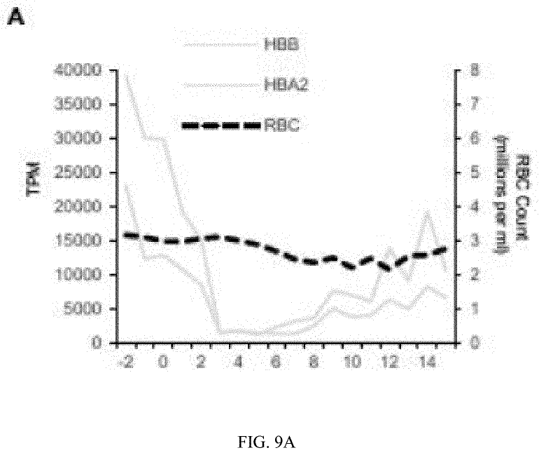

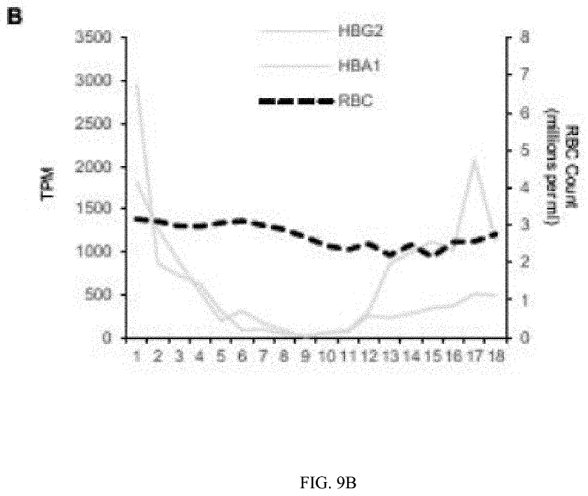

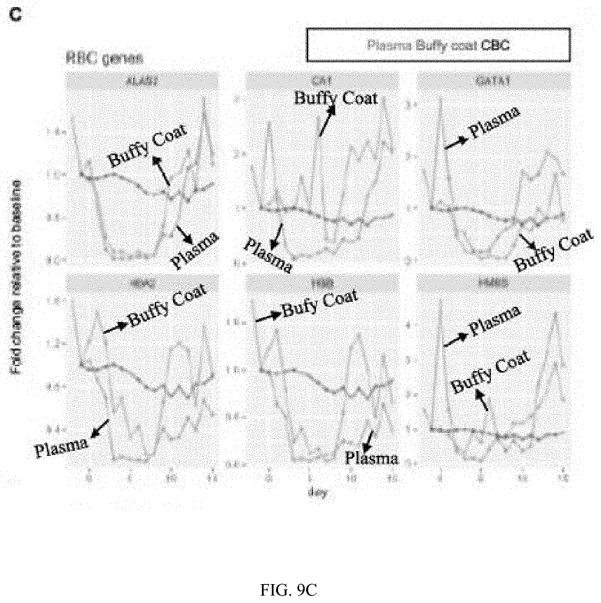

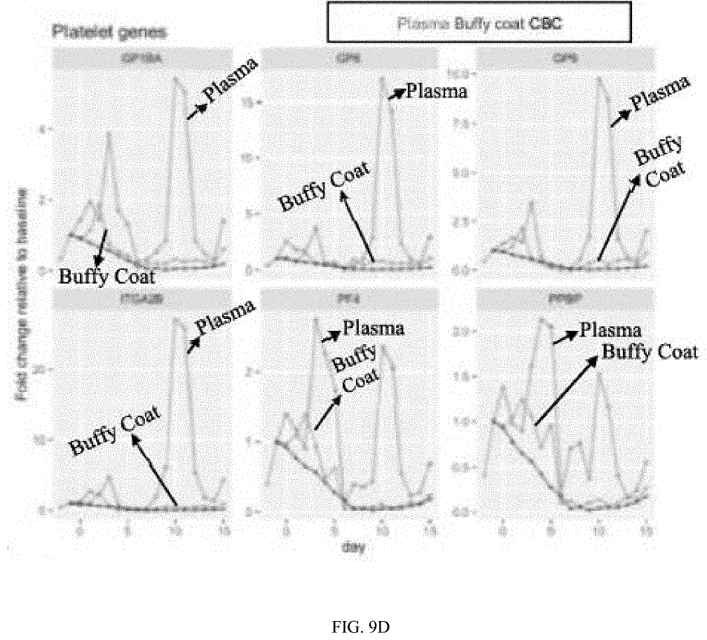

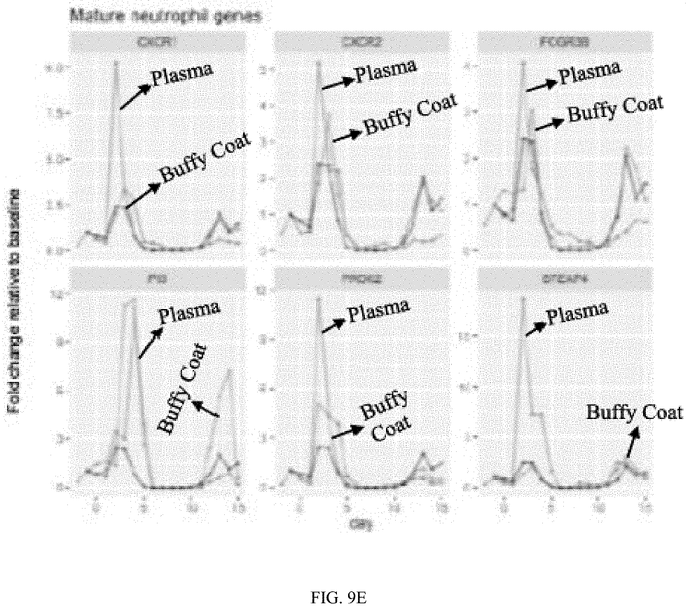

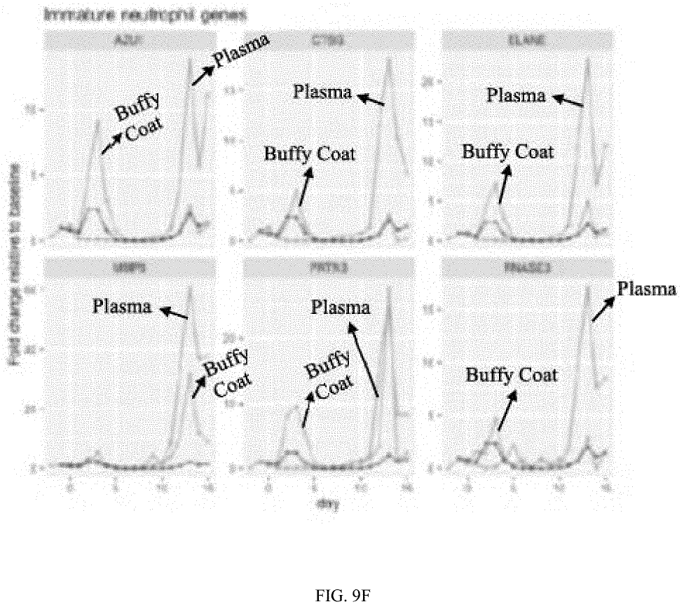

First, to clarify the relationship between erythrocyte circulating transcripts and RBCs, the levels of erythrocyte lineage-specific transcripts were examined in plasma and RBC counts were studied throughout the study. RBCs are the predominant cell type in circulation and are stable for ˜120 days in the bloodstream 21. Indeed, very little variation in RBC numbers was noticed in MM and AML patients during the duration of these studies (FIGS. 3C-3D, FIG. 8A). In contrast, erythrocyte-specific transcripts in cf-mRNA were rapidly reduced after chemotherapy-mediated BM ablation in all patients and recovered at later time points during BM reconstitution (FIGS. 3C-D, FIGS. 9A-9B, FIG. 8A). The dramatic discrepancy between RBC number and erythrocyte transcripts in cf-mRNA indicates that these transcripts do not derive from circulating mature RBCs. Therefore, erythrocyte transcripts derive from immature erythrocyte forms either in the BM or in circulation (reticulocytes). RNA-Seq analysis of paired buffy coat samples was performed of MM Patient 2 to gain further insights into the origin of these transcripts. The levels of erythrocyte specific genes in CC were reduced after chemotherapy, resembling the dynamics observed in cf-mRNA (FIG. 9C), and indicate that reticulocytes were the source of most erythrocyte transcripts in whole blood. However, transcripts like GATA1, a key transcriptional regulator of erythrocyte development, were clearly detectable in cf-mRNA earlier than in buffy coat during BM reconstitution (FIG. 9C), suggesting their BM origin. In conclusion, the data showed that erythrocyte transcripts derived from immature erythrocyte cells residing in the BM and circulating reticulocytes rather than from the highly abundant mature RBC.

To test whether the discrepancies between CBC and lineage-specific transcripts in circulation extend to other hematopoietic cell types, the dynamics of platelet counts, and megakaryocyte-specific transcripts were compared. In MM Patient 2, a dramatic increase in the levels of megakaryocyte-specific transcripts was detected in cf-mRNA by day 9-10 after transplant, prior to platelet count recovery, which occurs by day 12-13 (FIG. 3E). RNA-Seq from matched buffy coat samples showed that megakaryocyte transcript levels in CC mimic the dynamic of platelet counts throughout the study (FIG. 9C), and, unlike in cf-mRNA, no early recovery of megakaryocyte transcripts was detectable in CC during BM reconstitution. This disparity suggests that megakaryocyte transcripts detected in cf-mRNA during BM reconstitution were not derived from CC, but from the BM. Supporting this observation, in AML Patient 1 megakaryocyte transcripts in circulation decreased after BM ablation and recovered by day 9, foreshadowing the increase in platelet counts occurring by 12-13 (FIG. 3F). Strikingly, no recovery of this lineage occurred in cf-mRNA of AML Patient 2 (FIG. 8B). Follow-up BM biopsy confirmed lack of megakaryocyte development in this patient (Table 1), showing the specificity of the measured megakaryocyte signal. Thus, the data indicated that cf-mRNA reflected megakaryocyte transcriptional activity in the BM during its reconstitution.

Last, the kinetics of neutrophil counts and specific transcripts in circulation of MM and AML patients were examined during the therapy. In MM Patient 2, neutrophil counts showed two spikes, one right after transplant, likely due to the G-CSF treatment, which was followed by a rapid decrease due to BM ablation, and a second spike by day 12, indicating BM reconstitution (FIG. 3G). This resembled the overall dynamics of neutrophil-specific genes in cf-mRNA and in buffy coat during the procedure (FIG. 3G, FIG. 9E). However, while neutrophil transcripts in buffy coat and cf-mRNA peaked at a similar time to neutrophil counts during BM reconstitution, neutrophil precursor genes like CTSG increased about 2 days earlier in cf-mRNA, by day 8-9 after the stem cell transplant. Supporting this observation, the levels of progenitor neutrophil transcripts in plasma of all AML patients decreased after BM ablation and increased in cf-mRNA during BM reconstitution approximately five days earlier than the neutrophil counts (FIGS. 3H-J and FIG. 8D). These data further supported that progenitor neutrophil transcripts in circulation were not derived from CC, but rather reflected BM transcriptional activity of the granulocyte lineage, providing valuable information about transplant engraftment and BM reconstitution.

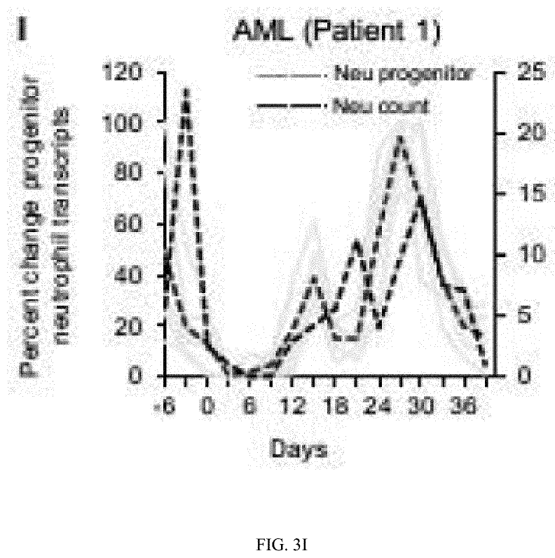

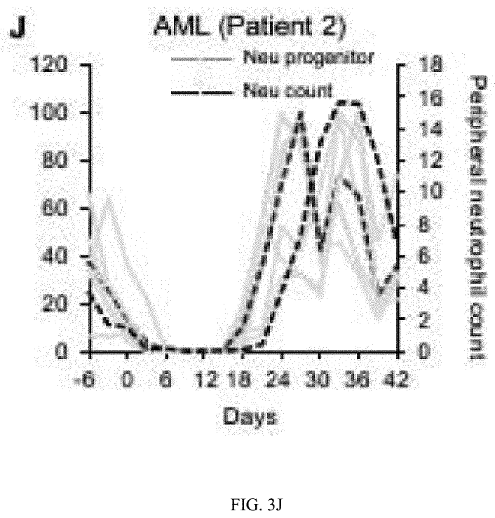

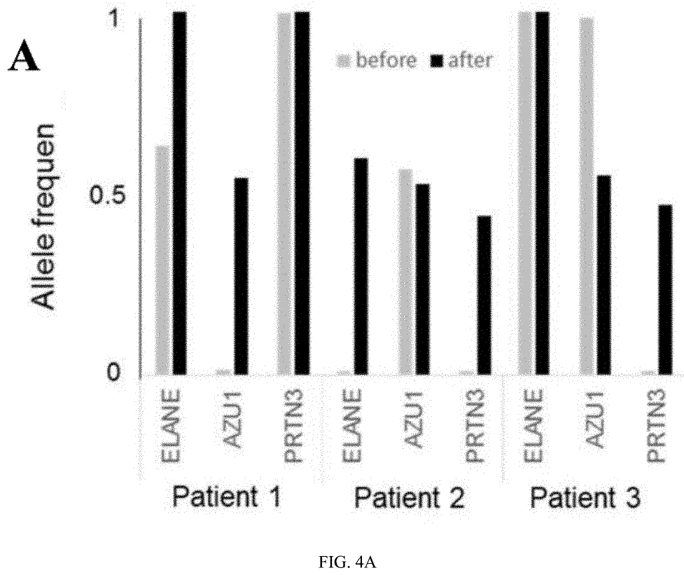

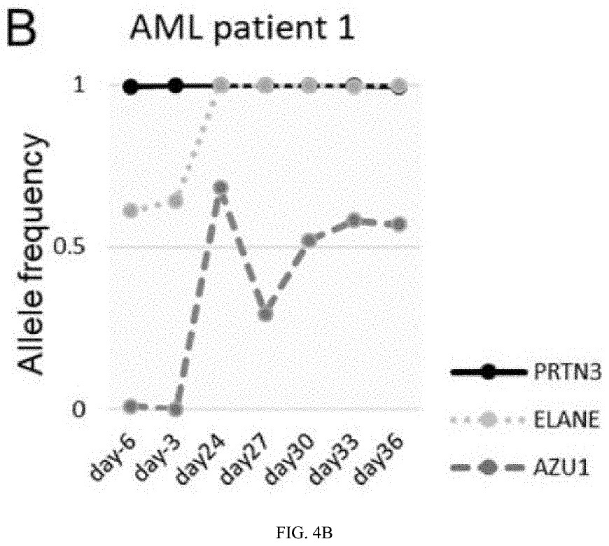

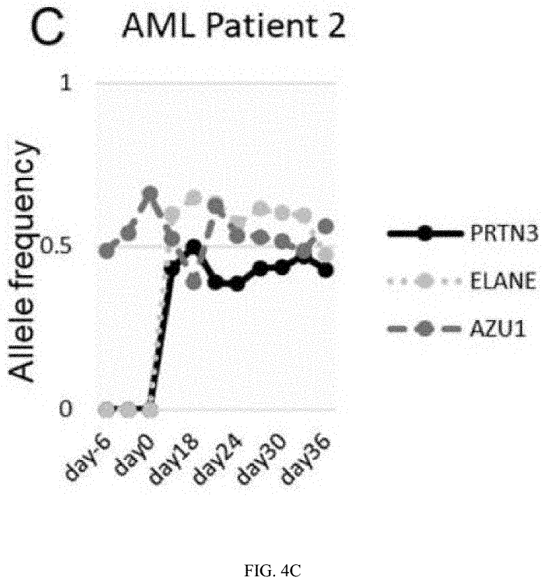

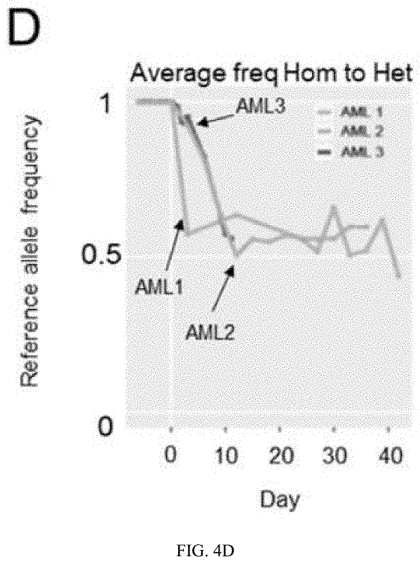

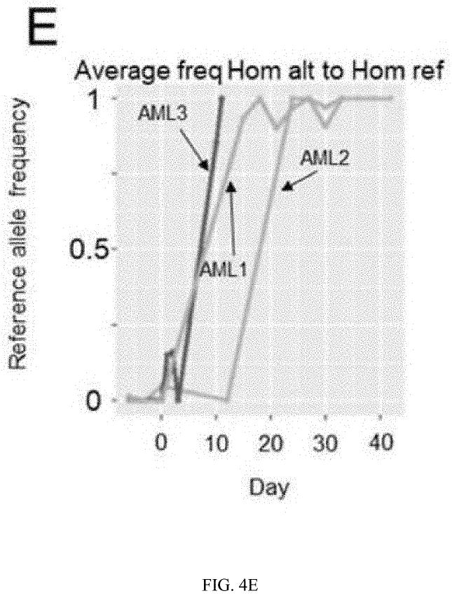

An orthogonal approach was also investigated to measure transplant engraftment using cf-mRNA from AML patients receiving allogeneic HSC transplants, in which genetic differences exist between host and donor cells. Using a reference data base of SNPs, host specific polymorphisms were identified in progenitor-neutrophil transcripts before the transplant (i.e., ELANE, AZU1, and PRTN3). After transplantation, these transcripts were substituted by new genetic variants from donor cells (FIG. 4A). Indeed, cf-mRNA profiling enabled monitoring of changes in these transcripts during therapeutic treatment of Patients 1 and 2 (FIGS. 4B-C). Combined analysis of all detected SNP from the host switching to a different genetic variant after transplant (i.e., from homozygous to heterozygous) indicates that multiple genetic differences may be identified in cf-mRNA to temporally monitor transplant engraftment (FIGS. 4D-E). Altogether, the data showed that cf-mRNA captured both genetic information and transcriptional activity from the BM, and enabled monitoring of transplant engraftment and BM reconstitution from donor cells.

### Example 12

### Lineage-Specific Transcriptional Activity Upon Stimulation with Growth Factors was Reflected in cf-mRNA

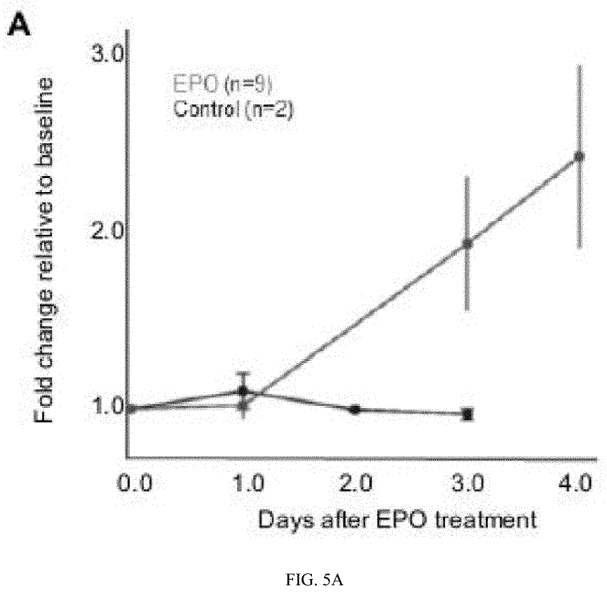

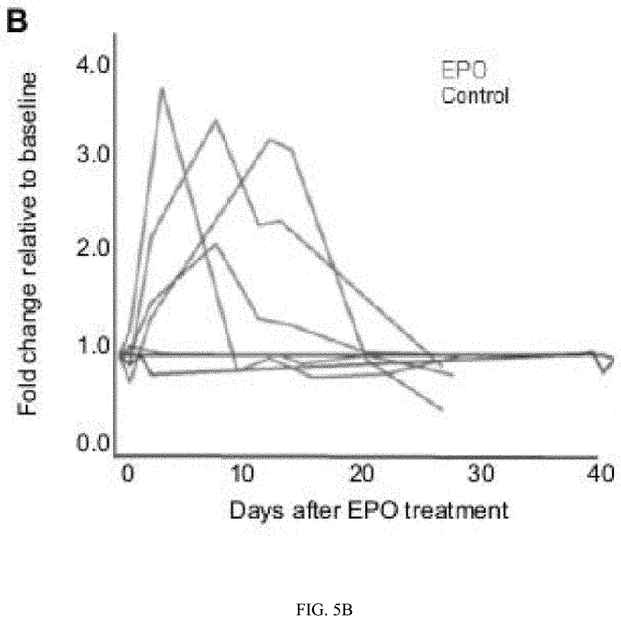

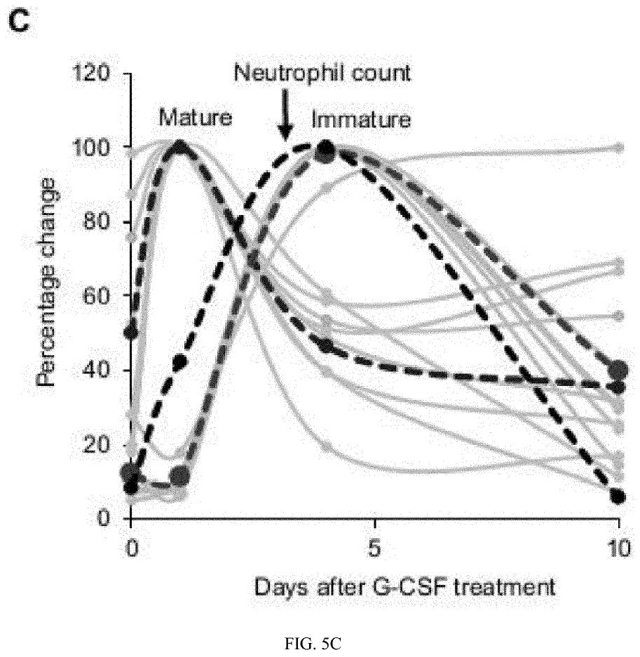

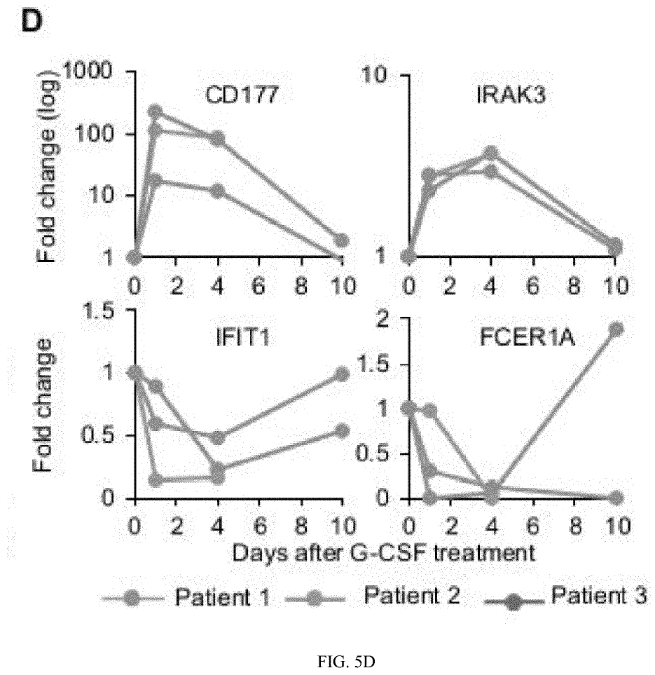

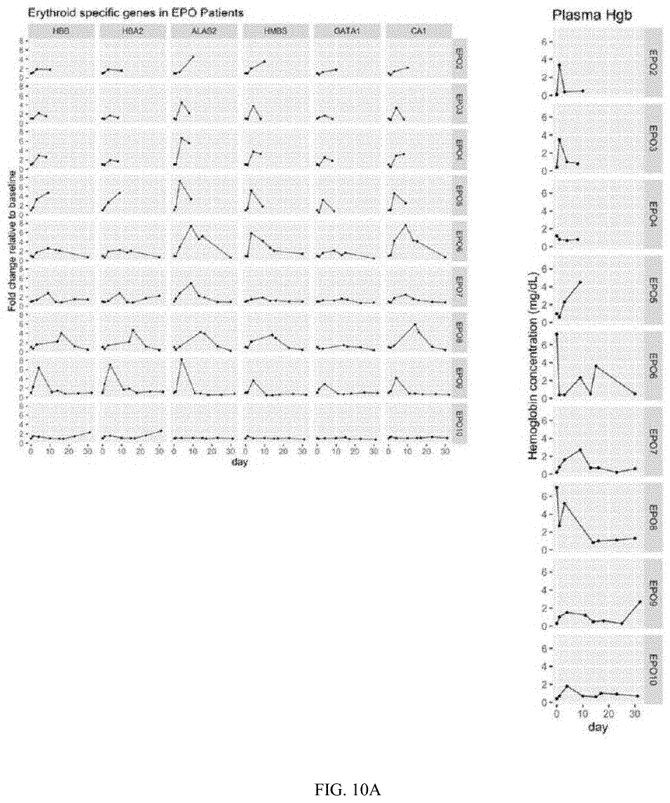

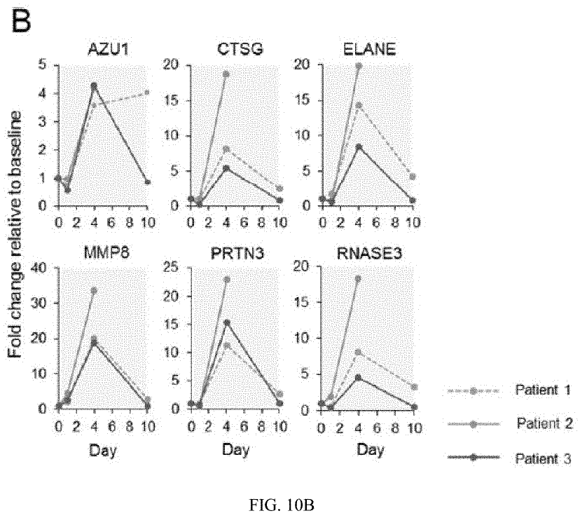

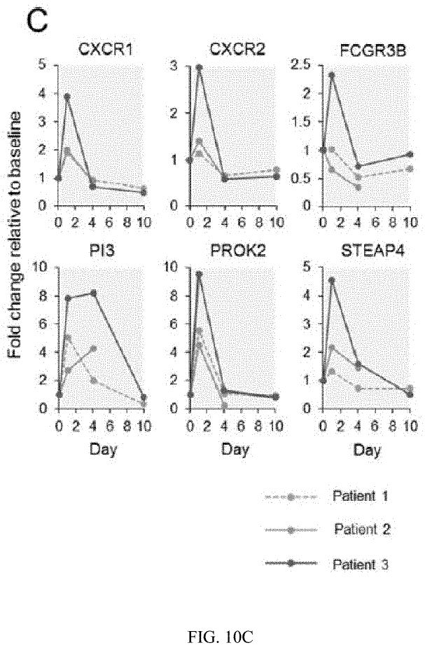

To evaluate the potential of cf-mRNA to monitor the activity of specific BM lineages after stimulation with growth factors, plasma samples from 9 patients were obtained with varying degrees of chronic kidney failure on chronic maintenance erythropoietin (EPO) therapy. EPO is a peptide hormone that specifically increases the rate of maturation and proliferation of erythrocytes in the BM. Samples were obtained prior to administration of EPO (day 0), and at several time points up to 30 days after treatment. Serum free hemoglobin and RBC number showed minor transient changes during the duration of the study. Unlike RBC counts, average levels of erythrocyte transcripts across 9 patients in cf-mRNA increased shortly after EPO treatment (FIG. 5A). The levels of erythrocyte transcripts continued to increase during the initial days after treatment compared to untreated control individuals (FIGS. 5A and 5B). Indeed, key erythropoietic developmental transcripts involved in heme biosynthesis (i.e., ALAS2, HBB, and HBA2) were induced in nearly all patients (8 out of 9 patients) (FIG. 10A). Further, 364 dysregulated genes were identified in plasma by day 4 after treatment with EPO (p<0.05). Analysis using IPA (www.qiagenbioinformatics.com/products/ingenuitypathway-analysis) showed “Heme biosynthesis II” as the top enriched pathway for these transcripts (p=1.4e-9), supporting the transcriptional induction of this cell lineage. 30 days after EPO treatment, erythrocyte transcripts returned to basal expression levels in these patients (FIG. 5B and FIGS. 10A-10C). Thus, the longitudinal studies indicated that cf-mRNA levels reflected specific transient stimulation of the erythroid lineage.

As another approach to study in vivo the changes in cf-mRNA upon perturbation of a cell lineage, samples from 3 healthy patients that received G-CSF treatment (granulocyte colony stimulating factor) were collected, a well-known pro-survival factor for neutrophilic granulocytes. Blood was drawn before the treatment and at 1, 4, and 10 days after G-CSF stimulation (the 10-day time point, and CBC could only be obtained for 2 patients). As expected, neutrophil count increased after G-CSF treatment, peaking at day 4, and returned to basal levels by day 10 (FIG. 5C). Neutrophil specific transcripts in plasma cf-mRNA showed a bimodal increase after G-CSF treatment for all patients (FIG. 5C and FIGS. 10B and 10C). Neutrophil progenitor-specific transcripts increased in cf-mRNA coinciding with the peak in neutrophil counts as a consequence of G-CSF-mediated mobilization of granulocytes from the BM into circulation (FIG. 5C, FIG. 10B). However, mature neutrophil transcripts rapidly increased in cf-mRNA one day after the treatment, foreshadowing the peak of neutrophil counts (FIG. 5C, FIG. 10C). This suggested a direct and transient transcriptional response of neutrophils to G-CSF. Indeed, transcripts previously reported both in vivo and in vitro to increase (e.g., IRAK3) or decrease (e.g., IFIT1) in neutrophils in response to G-CSF, followed the expected trend (FIG. 5D). Altogether, the results indicated that cf-mRNA reflected cell type-specific transcriptional responses to stimulation.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for monitoring a disease state of a subject's bone marrow, comprising:
obtaining a biological sample from said subject having said disease state; and
detecting cell-free mRNA (cf-mRNA) levels of a first plurality of cf-mRNAs derived from a plurality of cells resident or originated from said bone marrow corresponding to a first plurality of genes.

2.-63. (canceled)
